(19) United States
(12) Patent Application Publication
(10) Pub. No.: US 2007/0299102 A1
(43) Pub. Date: Dec. 27, 2007

(54) Diphenyl Ox-Indol-2-One Compounds and Their Use in the Treatment of Cancer

(75) Inventors: Jakob Felding, Charlottenlund (DK); Hans Christian Pedersen, Copenhagen V (DK); Christian Krog-Jensen, Rungsted Kyst (DK); Morten Praestegaard, Ballerup (DK); Steven Peter Butcher, Copenhagen (DK); Viggo Linde, Copenhagen (DK); Thomas Stephen Coulter, Oxfordshire (GB); Christian Montalbetti, Oxfordshire (GB); Mohammed Uddin, London (GB); Serge Reignier, Oxfordshire (GB)

Correspondence Address: DARBY & DARBY P.C., P.O. BOX 770, Church Street Station, New York, NY, 10008-0770, US

(73) Assignee: Topo Target A/S, Copenhagen O (DK)

(21) Appl. No.: 10/599,121

(22) PCT Filed: Apr. 8, 2005
(86) PCT No.: PCT/DK05/00244

§ 371 Date: Jun. 1, 2007

(30) Foreign Application Priority Data
Aug. 4, 2004 (DK) PA 2004 00576
May 1, 2004 (DK) PA 2004 00693
Jul. 27, 2004 (DK) PA 2004 01153
Aug. 11, 2004 (DK) PA 2004 01216

(51) Int. Cl. A61K 31/40; A61K 31/44; A61P 43/00; C07D 209/36; C07D 221/02

(52) U.S. Cl. 514/299; 514/418; 546/183; 548/485

(57) ABSTRACT

The present invention relates to substituted 3,3-diphenyl-1,3-dihydro-indol-2-one compounds, and the use of such compounds for the preparation of a medicament for the treatment of cancer in a mammal. It is postulated that treatment of cancers is achieved in which inhibition of protein synthesis and/or inhibition of activation of the mTOR pathway is an effective method for reducing cell growth. Examples of such cancers are breast cancer, renal cancer, multiple myeloma, leukemia, glia blastoma, rhabdomyosarcoma, prostate, soft tissue sarcoma, colorectal sarcoma, gastric carcinoma, head and neck squamous cell carcinoma, uterine, cervical, melanoma, lymphoma, and pancreatic cancer. A particular subclass of compounds are represented by the formula (II) 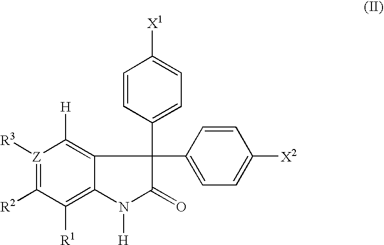
wherein at least one of X¹ and X² is a heteroatom substituent, e.g. 6-chloro-3,3-bis-(4-hydroxy-phenyl)-7-methyl-1,3-dihydro-indol-2-one.

### FIELD OF THE INVENTION

The present invention relates to substituted 3,3-diphenyl-1,3-dihydro-indol-2-one compounds, and the use of such compounds for the preparation of a medicament for the treatment of cancer in a mammal.

### BACKGROUND OF THE INVENTION

U.S. Pat. No. 1,624,675 describes O—O-diacyl derivatives of diphenolisatine and that these compounds possess laxative properties.

While inhibition of protein synthesis inhibits cell proliferation, highly proliferative cancer cells may be more sensitive than normal cells to protein synthesis inhibition because many oncogenes and growth regulatory proteins required for effective cell proliferation are encoded by inefficiently translated mRNAs, and are dependent on eukaryotic translation initiation factors (Aktas et al. (1998) Proc Natl Acad Sci 95, 8280 and references therein).

Protein synthesis is regulated in response to cell stress, which can be induced by environmental or physiological challenges (such as hypoxia, amino acid or nutrient deprivation), intracellular calcium load and protein glycosylation inhibition. For example, cell stressors such as clotrimazole, 3,3-diphenyloxindole, thapsigargin, tunicamycin and arsenite (Aktas et al. (1998) Proc Natl Acad Sci 95, 8280; Brewer et al. (1999) Proc Natl Acad Sci 96, 8505-8510; Harding et al. (2000) Molecular Cell 5, 897-904; Natarajan et al. (2004) J Med Chem 47, 1882-1885) act as protein translation initiation inhibitors, reducing both protein synthesis and cell proliferation.

The possibility that protein translation initiation inhibitors may have potential as anti-cancer drugs has been described previously (Aktas et al. (1998) Proc Natl Acad Sci 95; Natarajan et al. (2004) J. Med. Chem. 47, 1882-1885; Natarajan et al. (2004) J. Med. Chem. 47, 4979-4982). The Natarajan papers further disclose 3,3-diaryl-1,3-dihydroindol-2-ones which potentially inhibit protein translation.

Protein synthesis is also regulated by the mTOR pathway, providing another link to a nutrient and amino acid status (Harris & Lawrence (2003) ScienceSTKE (212) re15; Nave et al. (1999) Biochem J 344, 427; Beaunet et al. (2003) Biochem J 372, 555-566; Inoki et al. (2003) Cell 115, 577-590). This pathway is also linked to regulation of the protein translation initiation complex (Cherkasova & Hinnebusch (2003) Genes & Dev 17, 859-872; Kubota et al. (2003) J Biol Chem 278, 20457). Inhibition of mTOR signalling inhibits the proliferation of cancer cell lines (Noh et al. (2004) Clinical Cancer Research 10, 1013-1023; Yu et al. (2001) Endocrine-Related Cancer 8, 249-258), and has been proposed as a target for cancer therapy (Huang & Houghton (2003) Curr Opin Pharmacol 3, 371-377).

The lead compound among the 3,3-diaryl-1,3-dihydroindol-2-one compounds of the earliest Natarajan et al. paper (Natarajan et al. (2004) J. Med. Chem. 47, 1882-1885) is 3-(2-hydroxy-5-t-butyl-phenyl)-3-phenyl-1,3-dihydroindol-2-one.

US 2004/0242563 A1 discloses substituted diphenyl indanone, indane and indole compounds and analogues thereof useful for the treatment or prevention of diseases characterized by abnormal cell proliferation.

However, there is still a need for improved compounds capable of inhibiting the uncontrolled growth of cancer cells, in particular compounds exhibiting selective cancer cell proliferation inhibition.

### SUMMARY OF THE INVENTION

The present invention relates to the use of a hitherto sparsely studied subclass of 3,3-diphenyl-1,3-dihydroindol-2-one compounds in which the phenyl moieties are para-substituted via particular heteroatoms, in particular via oxygen atoms, in particular carrying hydroxy groups.

Thus, one aspect of the present invention relates to the use of a compound of the general formula (I) as defined herein for preparation of a medicament for the treatment of cancer in a mammal, cf. claim 1.

Another aspect of the present invention relates to a compound as defined herein for use as a medicament, with the proviso that the compound is not one selected from 3,3-bis-(4-hydroxy-phenyl)-1,3-dihydro-indol-2-one and acetic acid 4-[3-(4-acetoxy-phenyl)-2-oxo-2,3-dihydro-1H-indol-3-yl]-phenyl ester, claims 30.

A further aspect of the present invention relates to a novel compound of the general formula (I) or (II), cf. claims 31 and 32.

A still further aspect of the present invention relates to a pharmaceutical composition, cf. claim 33.

An even further aspect of the present invention relates to a method of treating a mammal suffering from or being susceptible to cancer.

### BRIEF DESCRIPTION OF THE FIGURES

FIG. 1: shows results from the cell proliferation studies using the compounds described in the Examples section corresponding to the following formula (III) (Example 2):

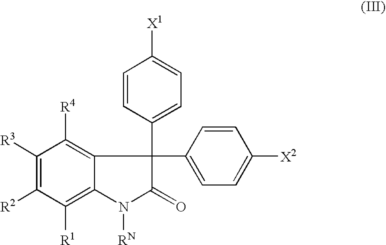

FIG. 2: shows results of the protein synthesis experiments using compound 3 in the MDA-468 and MDA-231 human breast cancer cell lines (Example 3).

FIG. 3: illustrates Translational Control pathways (from the Cell Signaling Technology catalogue 2003-2004).

FIG. 4: shows Western Blots on proteins involved in translational control using MDA-468 cells (24 hour compound incubation). 1: DMSO (0.08%); 2: Compound 3 (200 nM); 2: Compound 3 (2 μM); 4: other (2 μM); 5: Rapamycin (100 nM); and 6: LY294002 (10 μM) (Example 4).

FIG. 5: shows Western Blots on proteins involved in translational control comparing MDA-468 & MDA-231 cells (48 hours incubation). 1: DMSO (0.08%); 2: Compound 3 (200 nM); 4: other (2 μM); 5: Rapamycin (100 nM); and 6: LY294002 (10 μM) (Example 4).

FIG. 6: illustrates the results of PC3M human prostate cancer cell xenograft experiments using Compound 3 (Example 5).

FIG. 7: shows the effect of Compound 3 in a cell proliferation assay using a panel of human breast cancer cell lines in medium containing 1% FBS. PCTACT corresponds to growth inhibition relative to 50 μM terfenidine (100 PCTACT) (Example 6).

FIG. 8: shows the effect of Compound 3 on proliferation of the non-transformed human breast epithelial cell line MCF10A. PCTACT corresponds to growth inhibition relative to 50 μM terfenidine (100 PCTACT) (Example 6).

FIG. 9: shows the effect of Compound 3 in a cell proliferation assay using a panel of human breast cancer cell lines in medium containing 10% FBS. PCTACT corresponds to growth inhibition relative to 50 μM terfenidine (100 PCTACT) (Example 6).

FIG. 10: shows the effect of Compound 21 in a cell proliferation assay using a panel of breast cancer cell lines in medium containing 10% FBS (except MCF10A that is grown in serum-free MEGM medium). PCTACT corresponds to growth inhibition relative to 50 μM terfenidine (100 PCTACT) (Example 6).

FIG. 11: shows the effect of oxyphenisatine in a cell proliferation assay using a panel of breast cancer cell lines in medium containing 10% FBS (except MCF10A that is grown in serum-free MEGM medium). PCTACT corresponds to growth inhibition relative to 50 μM terfenidine (100 PCTACT) (Example 6).

FIG. 12: shows the effect of Compounds 3 and 21, and oxyphenisatine in a cell proliferation assay using a panel of prostate cancer cell lines in medium containing 10% FBS. PCTACT corresponds to growth inhibition relative to 50 μM terfenidine (100 PCTACT) (Example 6).

FIG. 13: shows the effect of Compounds 3 and 41 in a cell proliferation assay using PC3 prostate cancer cell lines in medium containing 10% FBS (Example 6).

FIG. 14: shows the results of the cell proliferation assay showing effect of Compound 3 on the colon cancer cell line Colo205 in medium containing 10% FBS. PCTACT corresponds to growth inhibition relative to 50 μM terfenidine (100 PCTACT) (Example 6).

FIG. 15: illustrates that Compound 3 reduces the rate of MDA-MB-468 tumour cell growth in xenograft experiments in a dose related manner when given as a monotherapy either by the PO or IV route. Furthermore, tumour regression is noted using the higher doses of Compound 3 (Example 7).

FIG. 16: illustrates that Compound 41 reduces the rate of MDA-MB-468 human breast cancer tumour cell growth in xenograft experiments and induces tumour regression at all doses tested when given as a monotherapy either by the PO or IV route. The effect is more pronounced than following administration of paclitaxel (Example 7).

FIG. 17: illustrates that Compound 41 reduces the rate of MCF-7 human breast cancer tumour cell growth in xenograft experiments and induces tumour regression at all doses tested when given as a monotherapy either by the PO or IV route. The effect is more pronounced than following administration of paclitaxel (Example 8).

FIG. 18: illustrates that Compound 3 activates caspase activity in most human breast cancer cell lines, indicating that the compound exhibits pro-apoptotic activity (Example 9).

### DETAILED DESCRIPTION OF THE INVENTION

Compounds for the Treatment of Cancer in a Mammal

One aspect of the present invention relates to particular compounds for the preparation of a medicament for the treatment of cancer in a mammal.

The term cancer is typically describing cell growth not under strict control. In one embodiment of the invention, treatment of cancers in which inhibition of protein synthesis and/or inhibition of activation of the mTOR pathway is an effective method for reducing cell growth. Examples of such cancers are breast cancer, renal cancer, multiple myeloma, leukemia, glio blastoma, rhabdomyosarcoma, prostate, soft tissue sarcoma, colorectal sarcoma, gastric carcinoma, head and neck squamous cell carcinoma, uterine, cervical, melanoma, lymphoma, and pancreatic cancer.

The useful compounds have the general formula (I), namely 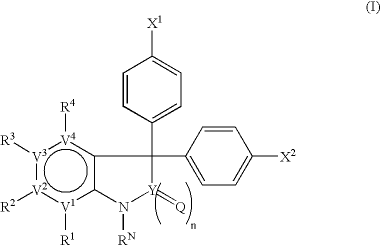
wherein
V¹, V², V³, and V⁴ independently are selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring;
R¹, R², R³, and R⁴, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
R¹, R², R³, and R⁴, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
or R¹ and R² together with the carbon atoms to which they are attached form a ring, e.g. an aromatic ring, a carbocyclic ring, a heterocyclic ring or a heteroaromatic ring, in particular an aromatic ring, a heterocyclic ring or a heteroaromatic ring;
X¹ and X² are independently selected from halogen, hydroxy, optionally substituted C₁₋₆-alkoxy, optionally substituted C₁₋₆-alkylcarbonyloxy, amino, mono- and di(C₁₋₆-alkyl)amino, C₁₋₆-alkylcarbonylamino, C₁₋₆-alkylsulphonylamino, mono- and di(C₁₋₆-alkyl)amino-carbonylamino, C₁₋₆-alkanoyloxy, mercapto, optionally substituted C₁₋₆-alkylthio, C₁₋₆-alkylsulfonyl, mono- and di(C₁₋₆-alkyl)aminosulfonyl, aryloxy, arylamino, heterocyclyloxy, heterocyclylamino, heteroaryloxy and heteroarylamino, where any C₁₋₆-alkyl as an amino or sulphur substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
>Y(=Q)ₙ is selected from >C═O, >C═S, >S═O and >S(═O)₂; and
R^{N} is selected from the group consisting of hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

Also included in the class of compounds of the formula (I) are pharmaceutically acceptable salts and prodrugs thereof.

One variant of the compounds of the formula (I) are those wherein each of the benzene rings to which X¹ and X² are attached further may be substituted with one, two, three or four fluoro atoms, in particular each benzene ring to which X¹ and X² are attached are substituted with two fluoro atoms in the ortho positions relative to the substituents X¹ and X², respectively.

Definitions

In the present context, the term “C₁₋₆-alkyl” is intended to mean a linear, cyclic or branched hydrocarbon group having 1 to 6 carbon atoms, such as methyl, ethyl, propyl, iso-propyl, pentyl, cyclopentyl, hexyl, cyclohexyl, and the term “C₁₋₄-alkyl” is intended to cover linear, cyclic or branched hydrocarbon groups having 1 to 4 carbon atoms, e.g. methyl, ethyl, propyl, iso-propyl, cyclopropyl, butyl, iso-butyl, tert-butyl, cyclobutyl.

Similarly, the term “C₂₋₆-alkenyl” is intended to cover linear, cyclic or branched hydrocarbon groups having 2 to 6 carbon atoms and comprising one unsaturated bond. Examples of alkenyl groups are vinyl, allyl, butenyl, pentenyl, hexenyl, heptenyl, octenyl, heptadecenyl. Preferred examples of alkenyl are vinyl, allyl, butenyl, especially allyl.

In the present context, i.e. in connection with the terms “alkyl”, “alkoxy”, and “alkenyl”, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkoxycarbonyl, C₁₋₆-alkylcarbonyl, formyl, aryl, aryloxy, arylamino, arylcarbonyl, aryloxycarbonyl, arylcarbonyloxy, arylaminocarbonyl, arylcarbonylamino, heteroaryl, heteroaryloxy, heteroarylamino, heteroarylcarbonyl, heteroaryloxycarbonyl, heteroarylcarbonyloxy, heteroarylaminocarbonyl, heteroarylcarbonylamino, heterocyclyl, heterocyclyloxy, heterocyclylamino, heterocyclylcarbonyl, heterocyclyloxycarbonyl, heterocyclylcarbonyloxy, heterocyclylaminocarbonyl, heterocyclylcarbonylamino, amino, mono- and di(C₁₋₆-alkyl)amino, carbamoyl, mono- and di(C₁₋₆-alkyl)aminocarbonyl, C₁₋₆-alkylcarbonylamino, cyano, guanidino, carbamido, C₁₋₆-alkyl-sulphonyl-amino, aryl-sulphonyl-amino, heteroaryl-sulphonyl-amino, C₁₋₆-alkanoyloxy, C₁₋₆-alkyl-sulphonyl, C₁₋₆-alkyl-sulphinyl, C₁₋₆-alkylsulphonyloxy, nitro, C₁₋₆-alkylthio, and halogen, where any aryl, heteroaryl and heterocyclyl may be substituted as specifically described below for aryl, heteroaryl and heterocyclyl, and any alkyl, alkoxy, and the like, representing substituents may be substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

Typically, the substituents are selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, heteroaryl, heteroaryloxy, heteroarylamino, heteroarylcarbonyl, heterocyclyl, heterocyclyloxy, heterocyclylamino, heterocyclylcarbonyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl, heteroaryl and heterocyclyl may be substituted as specifically described below for aryl, heteroaryl and heterocyclyl.

In some embodiments, substituents are selected from hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen.

The term “Halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

The term “heteroaryl” is intended to mean a fully or partially aromatic carbocyclic ring or ring system where one or more of the carbon atoms have been replaced with heteroatoms, e.g. nitrogen (═N— or —NH—), sulphur, and/or oxygen atoms. Examples of such heteroaryl groups are oxazolyl, isoxazolyl, thiazolyl, isothiazolyl, pyrrolyl, imidazolyl, pyrazolyl, pyridinyl, pyrimidinyl, pyrazinyl, pyridazinyl, triazinyl, coumaryl, furanyl, thienyl, quinolyl, benzothiazolyl, benzotriazolyl, benzodiazolyl, benzooxozolyl, phthalazinyl, phthalanyl, triazolyl, tetrazolyl, isoquinolyl, acridinyl, carbazolyl, dibenzazepinyl, indolyl, benzopyrazolyl, phenoxazonyl. Particularly interesting heteroaryl groups are benzimidazolyl, oxazolyl, isoxazolyl, thiazolyl, isothiazolyl, pyrrolyl, imidazolyl, pyrazolyl, pyridinyl, pyrimidinyl, pyrazinyl, pyridazinyl, furyl, thienyl, quinolyl, triazolyl, tetrazolyl, isoquinolyl, indolyl in particular benzimidazolyl, pyrrolyl, imidazolyl, pyridinyl, pyrimidinyl, furyl, thienyl, quinolyl, tetrazolyl, and isoquinolyl.

The term “heterocyclyl” is intended to mean a non-aromatic carbocyclic ring or ring system where one or more of the carbon atoms have been replaced with heteroatoms, e.g. nitrogen (═N— or —NH—), sulphur, and/or oxygen atoms. Examples of such heterocyclyl groups (named according to the rings) are imidazolidine, piperazine, hexahydropyridazine, hexahydropyrimidine, diazepane, diazocane, pyrrolidine, piperidine, azepane, azocane, aziridine, azirine, azetidine, pyrroline, tropane, oxazinane (morpholine), azepine, dihydroazepine, tetrahydroazepine, and hexahydroazepine, oxazolane, oxazepane, oxazocane, thiazolane, thiazinane, thiazepane, thiazocane, oxazetane, diazetane, thiazetane, tetrahydrofuran, tetrahydropyran, oxepane, tetrahydrothiophene, tetrahydrothiopyrane, thiepane, dithiane, dithiepane, dioxane, dioxepane, oxathiane, oxathiepane. The most interesting examples are tetrahydrofuran, imidazolidine, piperazine, hexahydropyridazine, hexahydropyrimidine, diazepane, diazocane, pyrrolidine, piperidine, azepane, azocane, azetidine, tropane, oxazinane (morpholine), oxazolane, oxazepane, thiazolane, thiazinane, and thiazepane, in particular tetrahydrofuran, imidazolidine, piperazine, hexahydropyridazine, hexahydropyrimidine, diazepane, pyrrolidine, piperidine, azepane, oxazinane (morpholine), and thiazinane.

In the present context, i.e. in connection with the terms “aryl”, “heteroaryl”, “heterocyclyl” and the like (e.g. “aryloxy”, “heterarylcarbonyl”, etc.), the term “optionally substituted” is intended to mean that the group in question may be substituted one or several times, preferably 1-5 times, in particular 1-3 times, with group(s) selected from hydroxy (which when present in an enol system may be represented in the tautomeric keto form), C₁₋₆-alkyl, C₁₋₆-alkoxy, C₂₋₆-alkenyloxy, oxo (which may be represented in the tautomeric enol form), carboxy, C₁₋₆-alkoxycarbonyl, C₁₋₆-alkylcarbonyl, formyl, aryl, aryloxy, arylamino, aryloxy-carbonyl, arylcarbonyl, heteroaryl, heteroarylamino, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, cyano, guanidino, carbamido, C₁₋₆-alkanoyloxy, C₁₋₆-alkyl-sulphonyl-amino, aryl-sulphonyl-amino, heteroaryl-sulphonyl-amino, C₁₋₆-alkyl-sulphonyl, C₁₋₆-alkyl-sulphinyl, C₁₋₆-alkylsulphonyloxy, nitro, sulphanyl, amino, amino-sulfonyl, mono- and di(C₁₋₆-alkyl)amino-sulfonyl, dihalogen-C₁₋₄-alkyl, trihalogen-C₁₋₄-alkyl, halogen, where aryl and heteroaryl representing substituents may be substituted 1-3 times with C₁₋₄-alkyl, C₁₋₄-alkoxy, nitro, cyano, amino or halogen, and any alkyl, alkoxy, and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

Typically, the substituents are selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocarbonyl, C₁₋₆-alkylcarbonylamino, guanidino, carbamido, C₁₋₆-alkyl-sulphonyl-amino, aryl-sulphonyl-amino, heteroaryl-sulphonyl-amino, C₁₋₆-alkyl-sulphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. In some embodiments, the substituents are selected from C₁₋₆-alkyl, C₁₋₆-alkoxy, amino, mono- and di(C₁₋₆-alkyl)amino, sulphanyl, carboxy or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

The term “prodrug” used herein is intended to mean a derivative of a compound of the formula (I) which—upon exposure to physiological conditions—will liberate a compound of the formula (I) which then will be able to exhibit the desired biological action. Examples of prodrugs are esters (carboxylic acid ester, phosphate esters, sulphuric acid esters, etc.), acid labile ethers, acetals, ketals, etc.

The term “pharmaceutically acceptable salts” is intended to include acid addition salts and basic salts. Illustrative examples of acid addition salts are pharmaceutically acceptable salts formed with non-toxic acids. Exemplary of such organic salts are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic, and theophylline acetic acids, as well as the 8-halotheophyllines, for example 8-bromotheophylline. Exemplary of such inorganic salts are those with hydrochloric, hydrobromic, sulfuric, sulfamic, phosphoric, and nitric acids. Examples of basic salts are salts where the (remaining) counter ion is selected from alkali metals, such as sodium and potassium, alkaline earth metals, such as calcium, and ammonium ions (⁺N(R)₃R′, where R and R′ independently designates optionally substituted C₁₋₆-alkyl, optionally substituted C₂₋₆-alkenyl, optionally substituted aryl, or optionally substituted heteroaryl). Pharmaceutically acceptable salts are, e.g., those described in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology. Thus, the term “an acid addition salt or a basic salt thereof” used herein is intended to comprise such salts. Furthermore, the compounds as well as any intermediates or starting materials may also be present in hydrate form.

### EMBODIMENTS

The function of V¹, V², V³, and V⁴ is mainly believed to be of sterical character, i.e. determinative for the orientation of the groups R¹-R⁴. It is, however, also believed that the selection of a heteroatom as one or more of V¹, V², V³, and V⁴ may create dipole interactions with other entities and thereby have influence on, e.g., the solubility of the compounds of the general formula (I).

V¹, V², V³, and V⁴ are independently selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring. Particularly useful examples of such aromatic rings and heteroaromatic rings are those selected from a benzene ring, a thiophene ring (V¹═S, V²═V³═C(−) and V⁴=bond; V²═S, V¹═V³═C(−) and V⁴=bond; or V³═S, V¹═V²═C(−) and V⁴=bond), a furan ring (V¹═O, V²═V³═C(−) and V⁴=bond; V²═O, V¹═V³═C(−) and V⁴=bond; or V³═O, V¹═V²═C(−) and V⁴=bond), a pyrazole ring (V¹═N(−), V²═N, V³═C(−) and V⁴=bond; V¹═N, V²═N(−), V³═C(−) and V⁴=bond), an imidazole ring (V¹═N(−), V²═C(−), V³═N and V⁴=bond; V¹═N, V²═C(−), V³═N(−) and V⁴=bond), a pyridine ring (V¹═N, V²═V³═V⁴═C(−); V²═N, V¹═V³═V⁴═C(−); V³═N, V¹═V²═V⁴═C(−) and V⁴═N, V¹═V²═V³═C(−)), a pyrimidine ring (V¹═V³═N, V²═V⁴═C(−); V²═V⁴═N, V¹═V³═C(−)), pyrazines (V¹═V⁴═N, V²═V³═C(−)), a pyridazine ring (V¹═V²═N, V³═V⁴═C(−); V²═V³═N, V¹═V⁴═C(−); V³═V⁴═N, V¹═V²═C(−)), a thiazole ring (V¹═N, V²═C(−), V³═S, V⁴=bond; V¹═S, V²═C(−), V³═N, V⁴=bond), and an isothiazole ring (V¹═N, V²═S, V³═C(−), V⁴=bond; V¹═S, V²═N, V³═C(−), V⁴=bond; V¹═C(−), V²═S, V³═N, V⁴=bond; V¹═C(−), V²═N, V³═S, V⁴=bond).

The meaning of V¹, V², V³ and V⁴ for each heteroaromatic ring is merely specified for the purpose of illustrating that various orientations of the heteroatoms are possible. Furthermore, it should be understood that the respective rings carry the substituents R¹, R², R³ and R⁴ (where applicable) in accordance with the general formula (I). Thus, specification of “C(−)” and “N(−)” as possible meanings of V¹, V², V³ and V⁴ is made for the purpose of describing that the atoms in question carry a substituent (which may be hydrogen). Specification of “N” means that the respective atoms do not carry an “R” substituent, i.e. the corresponding “R” substituent is absent.

In one embodiment, —V¹—V²—V³—V⁴— together with the atoms to which V¹ and V⁴ are attached form a ring selected from a benzene ring, a thiophene ring, a furan ring, a pyrazole ring, an imidazole ring, a pyridine ring, a pyrimidine ring, pyrazines, and a pyridazine ring, in particular from a benzene ring and a pyridine ring where the nitrogen atom represents V³ (see also the Examples). In accordance with the general formula (I), the respective ring (aromatic or heteroaromatic) carries the substituents R¹-R⁴ (where applicable).

The substituents R¹-R⁴ (where applicable) are believed to be at least partly responsible for the biological effect, e.g. the ability of the compounds to inhibit cell proliferation in cancer cells.

In one embodiment, R¹, R², R³, and R⁴ are, when attached to a carbon atom, independently selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s); and R¹, R², R³, and R⁴ are, when attached to a nitrogen atom, independently selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted.

More particularly, R¹, R², R³, and R⁴ are independently selected from hydrogen, halogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, amino, C₁₋₆-alkylcarbonylamino, C₁₋₆-alkylcarbonylamino, C₁₋₆-alkylsulphonylamino, mono- and di(C₁₋₆-alkyl)aminosulfonyl, and mono- and di(C₁₋₆-alkyl)amino, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), such as from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, amino, C₁₋₆-alkylcarbonylamino, C₁₋₆-alkylcarbonylamino, C₁₋₆-alkylsulphonylamino, mono- and di(C₁₋₆-alkyl)aminosulfonyl, and mono- and di(C₁₋₆-alkyl)amino, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

As an alternative to the above, R¹ and R² may in one embodiment together with the carbon atoms to which they are attached form a heterocyclic ring or a heteroaromatic ring; and in another embodiment, R¹ and R² may together with the carbon atoms to which they are attached form an aromatic ring or a carbocyclic ring.

In one particular variant, R¹ is selected from hydrogen, halogen, C₁₋₆-alkyl, trifluoromethyl and C₁₋₆-alkoxy, when V¹ is a carbon atom.

In a further variant, R² is selected from hydrogen, halogen, optionally substituted aryl, optionally substituted aryloxy, and optionally substituted heteroaryl, when V² is a carbon atom.

In a still further variant, R³ is selected from hydrogen, optionally substituted C₁₋₆-alkoxy, halogen, cyano, optionally substituted aryl, optionally substituted aryloxy, optionally substituted heteroaryl, amino, C₁₋₆-alkylcarbonylamino, C₁₋₆-alkylsulphonylamino, and mono- and di(C₁₋₆-alkyl)aminosulfonyl, when V³ is a carbon atom.

In an even still further variant, R⁴ is hydrogen, when V⁴ is a carbon atom.

According to the principal embodiment of the invention, it is believed that the substituents X¹ and X² must include a heteroatom directly bound to the phenyl ring, cf. the definition further above. (See also the alternative embodiment described further below.)

In one embodiment, X¹ and X² are independently selected from hydroxy, optionally substituted C₁₋₆-alkoxy, optionally substituted C₁₋₆-alkylcarbonyloxy, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, C₁₋₆-alkanoyloxy, and mono- and di(C₁₋₆-alkyl)aminosulfonyl, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

In a more preferred embodiment, X¹ and X² independently are selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl, such as from OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl, in particular X¹ and X² are independently selected from halogen, hydroxy, OAc, NH₂, NMe₂, NHAc, NHSO₂Me and NHCONMe₂, such as from hydroxy, OAc, NH₂, NMe₂, NHAc, NHSO₂Me and NHCONMe₂.

This being said, it is currently believed that X¹ and X² may be the same for both phenyl rings, i.e. X¹═X². This has the advantage that achiral compounds are achieved. In the pharmaceutical business, use of chiral drugs typically requires isolation of the individual stereoisomeric forms. Another advantage is seen in the synthesis route. A one-step introduction of the two PhX groups saves at least one synthesis step and associated time, and increases the overall yield of the preparation process.

Although not explicitly specified in the general formula (I), it is believed that introduction of fluoro atoms in the benzene rings may provide certain advantages. Thus as defined above, a variant of compounds are those wherein each of the benzene rings to which X¹ and X² are attached further may be substituted with one, two, three or four fluoro atoms, in particular each benzene ring to which X¹ and X² are attached are substituted with two fluoro atoms in the ortho positions relative to the substituents X¹ and X², respectively.

The structural element >Y(=Q)ₙ is not considered particularly critical. However, for synthetic reasons, it is preferred that Y is a carbon atom and Q is an oxygen atom, i.e. >Y(=Q)ₙ is >C═O. In the alternative, Y is a sulfur atom, n is 2, and each Q is an oxygen atom, i.e. >Y(=Q)ₙ is >S(═O)₂.

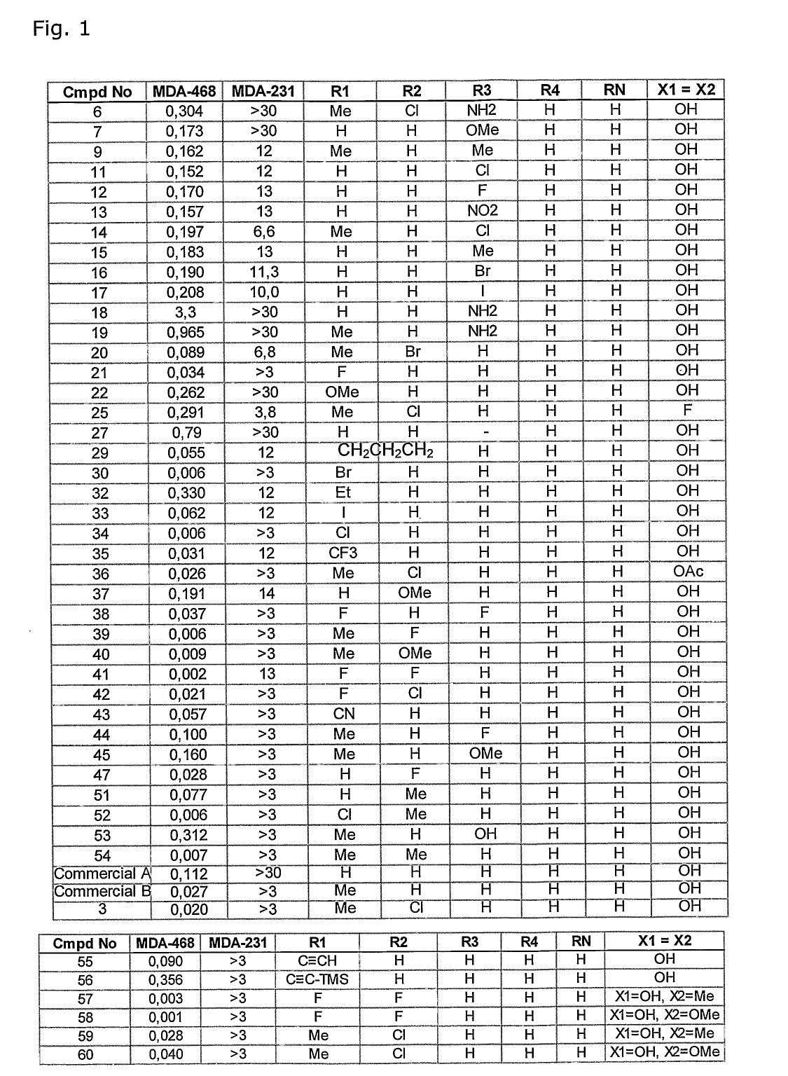

It is believed that R^{N} may be selected from a wide variety of substituents. However, it is currently believed that it may be advantageous if R^{N} is selected from hydrogen, C₁₋₆-alkyl, amino, and C₁₋₆-alkylcarbonylamino. Most preferred is the embodiments wherein R^{N} is hydrogen (see FIG. 1).

In view of the above, and in view of the current set of biological data, it is postulated that certain subclasses of compounds may exhibit particular advantages, cf. the subclasses defined in the following:

(a) One subclass of compounds are those wherein V¹, V², V³, V⁴ all are a carbon atom, >Y(=Q)ₙ is >C═O, and R^{N} is hydrogen.

In a first embodiment hereof, R⁴ is hydrogen; in particular, both of R³ and R⁴ are hydrogen.

In second embodiment within the subclass, which may be combined with the first embodiment, R¹ is C₁₋₄-alkyl and R² is halogen, e.g. R¹ is methyl and R² is chloro.

In a third embodiment within this subclass, which may be combined with the first embodiment, R¹ and R² together with the carbon atoms to which they are attached form a ring, e.g. an aromatic ring, a carbocyclic ring, a heterocyclic ring or a heteroaromatic ring, in particular an aromatic ring or a carbocyclic ring.

In a fourth embodiment within this subclass, which may be combined with the preceding embodiments, each of X¹ and X² independently are selected from halogen, hydroxy, C₁₋₄-alkoxy, amino, and dimethylamino.

In a fifth embodiment within this subclass, which may be combined with the first embodiment, R¹, R² and R⁴ all are hydrogen.

In a sixth embodiment within this subclass, which may be combined with the fifth embodiment, R³ is selected from hydrogen, halogen (such as fluoro, chloro, bromo, iodo), nitro, C₁₋₄-alkyl (such as methyl), C₁₋₄-alkoxy (such as methoxy), trifluoromethoxy, amino, carboxy, and dimethylaminocarbonyl, in particular hydrogen, halogen (such as fluoro, chloro, bromo, iodo), nitro, methyl, methoxy, and amino.

In a seventh embodiment within this subclass, which is combined with the fifth or sixth embodiment, each of X¹ and X² independently are selected from halogen, hydroxy, C₁₋₄-alkoxy, amino, and dimethylamino.

In an eighth embodiment within this subclass, R², R³ and R⁴ all are hydrogen.

In a ninth embodiment within this subclass, which may be combined with the eighth embodiment, R¹ is selected from fluoro, chloro, bromo, C₁₋₄-alkyl (such as methyl or tert-butyl), trifluoromethyl, C₁₋₄-alkoxy (such as methoxy), and dimethylaminocarbonyl.

In a tenth embodiment, which may be combined with any of the eighth and ninth embodiments, each of X¹ and X² independently are selected from halogen (such as fluoro) hydroxy, C₁₋₄-alkoxy (such as methoxy), amino, and dimethylamino.

In an eleventh embodiment series, which may be combined with the first embodiment, R¹ is selected from halogen (such as fluoro, chloro, bromo), C₁₋₄-alkyl (such as methyl or tert-butyl), trifluoromethyl, C₁₋₄-alkoxy (such as methoxy), and dimethylaminocarbonyl, R² is selected from hydrogen and halogen, and R³ is selected from hydrogen, halogen, C₁₋₄-alkyl (such as methyl), and amino; where R² and R³ are not both hydrogen.

Also preferred within this subclass and any of the embodiments are the variants, wherein X¹ and X² are the same.

(b) Another subclass of compounds are those wherein at least one of V¹, V², V³, and V⁴ is selected from a non-quaternary nitrogen atom, an oxygen atom, and a sulfur atom, and where V⁴ further may be selected from a bond, so that —V¹—V²—V³—V⁴— together with the atoms to which V¹ and V⁴ are attached form a heteroaromatic ring. In this case, the heteroaromatic ring is preferably selected from a pyridine ring and a pyrazole ring.

Within this subclass, it is further preferred that >Y(=Q)ₙ is >C═O and R^{N} is hydrogen. Also preferred are the embodiments, wherein X¹ and X² are the same.

A further aspect of the invention relates to the use of a 3,3-diphenyl-1,3-dihydro-indol-2-one type compound of the formula (IIa) 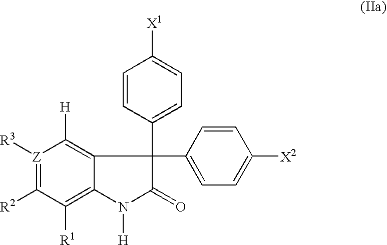
wherein
R¹ is selected from hydrogen, halogen, C₁₋₆-alkyl, trifluoromethyl and C₁₋₆-alkoxy;
R² is selected from hydrogen, halogen, optionally substituted aryl, optionally substituted aryloxy, and optionally substituted heteroaryl;
R³ is selected from hydrogen, optionally substituted C₁₋₆-alkoxy, halogen, cyano, and optionally substituted aryl, optionally substituted aryloxy, optionally substituted heteroaryl, amino, C₁₋₆-alkylcarbonylamino, C₁₋₆-alkylsulphonylamino, and mono- and di(C₁₋₆-alkyl)aminosulfonyl;
Z is CH or N; and
X¹ and X² are independently selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; and
pharmaceutically acceptable salts and prodrugs thereof (as defined further above);
for the preparation of a medicament for the treatment of cancer in a mammal.

As above, each of the benzene rings to which X¹ and X² are attached further may be substituted with one, two, three or four fluoro atoms, in particular each benzene ring to which X¹ and X² are attached are substituted with two fluoro atoms in the ortho positions relative to the substituents X¹ and X², respectively.

In one embodiment, X¹ and X² are independently selected from OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl.

In one variant which may be combined with the before-mentioned embodiments within this aspect, R¹ is selected from C₁₋₆-alkyl and C₁₋₆-alkoxy, such as from methyl, ethyl, isopropyl, methoxy, ethoxy and isopropoxy, in particular from methoxy, ethoxy and isopropoxy, or from methyl, ethyl, and isopropyl.

In another variant which may be combined with the before-mentioned embodiments and variants within this aspect, R² is selected from hydrogen, chloro, methoxy, dimethylamino, phenyl, phenoxy, optionally substituted thiophen-2-yl, and optionally substituted thiophen-3-yl.

In still another variant which may be combined with the before-mentioned embodiments and variants within this aspect, R³ is selected from hydrogen, methoxy, fluoro, chloro, cyano, phenyl, phenoxy, optionally substituted thiophen-2-yl, and optionally substituted thiophen-3-yl, amino, acetylamino, methylsulfonylamino, and dimethylaminosulfonyl.

In a still further variant, X¹ and X² independently are selected from halogen, hydroxy, OAc, NH₂, NMe₂, NHAc, NHSO₂Me and NHCONMe₂, such as from hydroxy, OAc, NH₂, NMe₂, NHAc, NHSO₂Me and NHCONMe₂.

Within this aspect, each X¹ and X² are preferably the same.

A still further aspect of the invention relates to the use of a 3,3-diphenyl-1,3-dihydro-indol-2-one type compound of the formula (IIb) 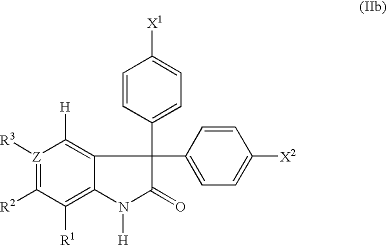
wherein
R¹, R², and R³, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s); and
R¹, R², and R³, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
or wherein R¹ and R² together with the carbon and/or nitrogen atoms to which they are attached form a heterocyclic ring, a heteroaromatic ring, an aromatic ring or a carbocyclic ring;
Z is CH or N; and
X¹ and X² are independently selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; and
pharmaceutically acceptable salts and prodrugs thereof;
for the preparation of a medicament for the treatment of cancer in a mammal.

In one embodiment, R¹, R², and R³ independently are selected from hydrogen, halogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, amino, C₁₋₆-alkylcarbonylamino, C₁₋₆-alkylcarbonylamino, C₁₋₆-alkylsulphonylamino, mono- and di(C₁₋₆-alkyl)aminosulfonyl, nitro, cyano, and mono- and di(C₁₋₆-alkyl)amino, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s); preferably, R¹, R², and R³ independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, amino, C₁₋₆-alkylcarbonylamino, C₁₋₆-alkylcarbonylamino, C₁₋₆-alkylsulphonylamino, mono- and di(C₁₋₆-alkyl)aminosulfonyl, nitro, cyano, and mono- and di(C₁₋₆-alkyl)amino, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s).

An another embodiment, R¹ and R² together with the carbon atoms to which they are attached form a heterocyclic ring or a heteroaromatic ring.

In still another embodiment, R¹ and R² together with the carbon atoms to which they are attached form an aromatic ring or a carbocyclic ring.

In preferred variants of the above aspect and embodiments, Z is CH.

In further preferred variants of the above aspect, embodiments and variant, X¹ and X² are independently selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; in particular X¹ and X² are independently selected from halogen, OR⁶, and OCOR⁵, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl.

In further preferred variants of the above aspect, embodiments and variants, R¹ and R² independently are selected from hydrogen, halogen, C₁₋₆-alkyl, cyano, trifluoromethyl and C₁₋₆-alkoxy; R³ is selected from hydrogen, C₁₋₆-alkoxy, halogen, nitro, cyano, and amino.

### ALTERNATIVE EMBODIMENTS

An alternative subclass of compound applicable for the use defined hereinabove, is essentially as defined above for the compounds of Formula I, but with the modification that X¹ and X² are not the same. In a main embodiment hereof, one of X¹ and X² is as defined for X¹ and X² above, whereas the other of X¹ and X² is a carbon-substituent, e.g. a substituent selected from optionally substituted C₁₋₆-alkyl, optionally substituted C₂₋₆-alkenyl, carboxy, optionally substituted C₁₋₆-alkoxycarbonyl, optionally substituted C₁₋₆-alkylcarbonyl, formyl, carbamoyl, mono- and di(C₁₋₆-alkyl)aminocarbonyl, cyano, aryl, arylcarbonyl, heterocyclyl, heterocyclylcarbonyl, heteroaryl, heteroarylcarbonyl, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted. The remaining substituents are as defined above.

Thus, a further aspect of the invention relates to the use of a 3,3-diphenyl-1,3-dihydro-indol-2-one type compound of the formula (IIc) 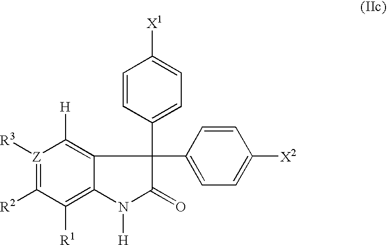
wherein
R¹ is selected from hydrogen, halogen, C₁₋₆-alkyl, trifluoromethyl and C₁₋₆-alkoxy;
R² is selected from hydrogen, halogen, optionally substituted aryl, optionally substituted aryloxy, and optionally substituted heteroaryl;
R³ is selected from hydrogen, optionally substituted C₁₋₆-alkoxy, halogen, cyano, and optionally substituted aryl, optionally substituted aryloxy, optionally substituted heteroaryl, amino, C₁₋₆-alkylcarbonylamino, C₁₋₆-alkylsulphonylamino, and mono- and di(C₁₋₆-alkyl)aminosulfonyl;
Z is CH or N; and
one of X¹ and X² is selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; and the other of X¹ and X² is selected from optionally substituted C₁₋₆-alkyl, optionally substituted C₂₋₆-alkenyl, carboxy, optionally substituted C₁₋₆-alkoxycarbonyl, optionally substituted C₁₋₆-alkylcarbonyl, formyl, carbamoyl, mono- and di(C₁₋₆-alkyl)aminocarbonyl, cyano, aryl, arylcarbonyl, heterocyclyl, heterocyclylcarbonyl, heteroaryl, heteroarylcarbonyl, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; and
pharmaceutically acceptable salts and prodrugs thereof (as defined further above);
for the preparation of a medicament for the treatment of cancer in a mammal.

The embodiments defined for the compound (IIa) above also apply for the compound of the Formula (IIc), mutatis mutantis.

A still further aspect of the invention relates to the use of a 3,3-diphenyl-1,3-dihydro-indol-2-one type compound of the formula (IId) 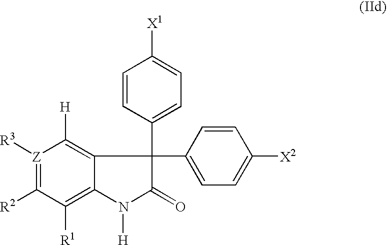
wherein
R¹, R², and R³, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s); and
R¹, R², and R³, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
or wherein R¹ and R² together with the carbon and/or nitrogen atoms to which they are attached form a heterocyclic ring, a heteroaromatic ring, an aromatic ring or a carbocyclic ring;
Z is CH or N; and
one of X¹ and X² is selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; and the other of X¹ and X² is selected from optionally substituted C₁₋₆-alkyl, optionally substituted C₂₋₆-alkenyl, carboxy, optionally substituted C₁₋₆-alkoxycarbonyl, optionally substituted C₁₋₆-alkylcarbonyl, formyl, carbamoyl, mono- and di(C₁₋₆-alkyl)aminocarbonyl, cyano, aryl, arylcarbonyl, heterocyclyl, heterocyclylcarbonyl, heteroaryl, heteroarylcarbonyl, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted; and
pharmaceutically acceptable salts and prodrugs thereof;
for the preparation of a medicament for the treatment of cancer in a mammal.

The embodiments defined for the compound (IIb) above also apply for the compound of the Formula (IId), mutatis mutantis.

Presently very interesting compounds of the formula I are those listed in the following as Items 1 to 225:
1 5-Amino-6-chloro-3,3-bis-(4-hydroxy-phenyl)-7-methyl-1,3-dihydro-indol-2-one
2 5-Chloro-3,3-bis-(4-hydroxy-phenyl)-7-methyl-1,3-dihydro-indol-2-one
3 5-Fluoro-3,3-bis-(4-hydroxy-phenyl)-1,3-dihydro-indol-2-one
4 3,3-Bis-(4-hydroxy-phenyl)-5-nitro-1,3-dihydro-indol-2-one
5 3,3-Bis-(4-hydroxy-phenyl)-7-methyl-1,3-dihydro-pyrrolo[3,2-c]pyridin-2-one
6 6-Bromo-3,3-bis-(4-hydroxy-phenyl)-1,3-dihydro-pyrrolo[3,2-c]pyridin-2-one
7 6-Bromo-3,3-bis-(4-hydroxy-phenyl)-7-methyl-1,3-dihydro-pyrrolo[3,2-c]pyridin-2-one
8 6-Bromo-3,3-bis-(4-hydroxy-phenyl)-5,7-dimethyl-1,3-dihydro-indol-2-one
9 6-Bromo-3,3-bis-(4-hydroxy-phenyl)-7-methyl-2-oxo-2,3-dihydro-1H-indole-5-carbonitrile
10 6-Bromo-3,3-bis-(4-hydroxy-phenyl)-5-methoxy-7-methyl-1,3-dihydro-indol-2-one
11 6-Bromo-3,3-bis-(4-hydroxy-phenyl)-7-methoxy-1,3-dihydro-pyrrolo[3,2-c]pyridin-2-one;
12 6-Bromo-7-ethyl-3,3-bis-(4-hydroxy-phenyl)-1,3-dihydro-pyrrolo[3,2-c]pyridin-2-one
13 6-Bromo-7-ethyl-3,3-bis-(4-hydroxy-phenyl)-5-methyl-1,3-dihydro-indol-2-one
14 6-Bromo-5-ethyl-3,3-bis-(4-hydroxy-phenyl)-7-methyl-1,3-dihydro-indol-2-one
15 6-Bromo-7-ethyl-3,3-bis-(4-hydroxy-phenyl)-2-oxo-2,3-dihydro-1H-indole-5-carbonitrile
16 6-Bromo-7-ethyl-3,3-bis-(4-hydroxy-phenyl)-5-methoxy-1,3-dihydro-indol-2-one
17 6-Chloro-3,3-bis-(4-hydroxy-phenyl)-1,3-dihydro-pyrrolo[3,2-c]pyridin-2-one
18 6-Chloro-3,3-bis-(4-hydroxy-phenyl)-7-methyl-1,3-dihydro-pyrrolo[3,2-c]pyridin-2-one
19 6-Chloro-3,3-bis-(4-hydroxy-phenyl)-5,7-dimethyl-1,3-dihydro-indol-2-one
20 6-Chloro-3,3-bis-(4-hydroxy-phenyl)-7-methyl-2-oxo-2,3-dihydro-1H-indole-5-carbonitrile
21 6-Chloro-3,3-bis-(4-hydroxy-phenyl)-5-methoxy-7-methyl-1,3-dihydro-indol-2-one
22 6-Chloro-3,3-bis-(4-hydroxy-phenyl)-7-methoxy-1,3-dihydro-pyrrolo[3,2-c]pyridin-2-one
23 6-Chloro-7-ethyl-3,3-bis-(4-hydroxy-phenyl)-1,3-dihydro-pyrrolo[3,2-c]pyridin-2-one
24 6-Chloro-7-ethyl-3,3-bis-(4-hydroxy-phenyl)-5-methyl-1,3-dihydro-indol-2-one
25 6-Chloro-5-ethyl-3,3-bis-(4-hydroxy-phenyl)-7-methyl-1,3-dihydro-indol-2-one
26 6-Chloro-7-ethyl-3,3-bis-(4-hydroxy-phenyl)-2-oxo-2,3-dihydro-1H-indole-5-carbonitrile
27 6-Chloro-7-ethyl-3,3-bis-(4-hydroxy-phenyl)-5-methoxy-1,3-dihydro-indol-2-one
28 6-Chloro-3,3-bis-(4-hydroxy-phenyl)-5-methyl-7-methoxy-1,3-dihydro-indol-2-one;
29 6-Chloro-3,3-bis-(4-hydroxy-phenyl)-7-methoxy-2-oxo-2,3-dihydro-1H-indole-5-carbonitrile;
30 6-Chloro-3,3-bis-(4-hydroxy-phenyl)-7-methoxy-1,3-dihydro-pyrrolo[3,2-c]pyridin-2-one;
31 6-Chloro-3,3-bis-(4-hydroxy-phenyl)-7-methoxy-5-methyl-1,3-dihydro-indol-2-one;
32 6-Chloro-5-ethyl-3,3-bis-(4-hydroxy-phenyl)-7-methoxy-1,3-dihydro-indol-2-one;
33 6-Chloro-3,3-bis-(4-hydroxy-phenyl)-5,7-dimethoxy-1,3-dihydro-indol-2-one;
34 N-{4-[3-(4-Acetylamino-phenyl)-5-chloro-7-methyl-2-oxo-2,3-dihydro-1H-indol-3-yl]-phenyl}-acetamide;
35 N-{4-[5-Chloro-3-(4-methanesulfonylamino-phenyl)-7-methyl-2-oxo-2,3-dihydro-1H-indol-3-yl]-phenyl}-methanesulfonamide
36 N-{4-[3-(4-Acetylamino-phenyl)-6-chloro-7-methyl-2-oxo-2,3-dihydro-1H-indol-3-yl]-phenyl}-acetamide;
37 N-{4-[6-Chloro-3-(4-methanesulfonylamino-phenyl)-7-methyl-2-oxo-2,3-dihydro-1H-indol-3-yl]-phenyl}-methanesulfonamide;
38 N-{4-[3-(4-Acetylamino-phenyl)-5-chloro-7-methoxy-2-oxo-2,3-dihydro-1H-indol-3-yl]-phenyl}-acetamide;
39 N-{4-[5-Chloro-3-(4-methanesulfonylamino-phenyl)-7-methoxy-2-oxo-2,3-dihydro-1H-indol-3-yl]-phenyl}-methanesulfonamide;
40 N-{4-[3-(4-Acetylamino-phenyl)-6-chloro-7-methoxy-2-oxo-2,3-dihydro-1H-indol-3-yl]-phenyl}-acetamide; and
41 N-{4-[6-Chloro-3-(4-methanesulfonylamino-phenyl)-7-methoxy-2-oxo-2,3-dihydro-1H-indol-3-yl]-phenyl}-methanesulfonamide
42 2-Chloro-6,6-bis-(4-hydroxy-phenyl)-3-methyl-4,6-dihydro-3H-pyrrolo[2,3-d]imidazol-5-one
43 Acetic acid 4-[6-(4-acetoxy-phenyl)-2-chloro-3-methyl-5-oxo-3,4,5,6-tetrahydro-pyrrolo[2,3-d]imidazol-6-yl]-phenyl ester
44 6,6-Bis-(4-amino-phenyl)-2-chloro-3-methyl-4,6-dihydro-3H-pyrrolo[2,3-d]imidazol-5-one
45 2-Chloro-6,6-bis-(4-dimethylamino-phenyl)-3-methyl-4,6-dihydro-3H-pyrrolo[2,3-d]imidazol-5-one
46 N-{4-[6-(4-Acetylamino-phenyl)-2-chloro-3-methyl-5-oxo-3,4,5,6-tetrahydro-pyrrolo[2,3-d]Imidazol-6-yl]-phenyl}-acetamide
47 N-{4-[2-Chloro-6-(4-methanesulfonylamino-phenyl)-3-methyl-5-oxo-3,4,5,6-tetrahydro-pyrrolo[2,3-d]imidazol-6-yl]-phenyl}-methanesulfonamide
48 4,4-Bis-(4-hydroxy-phenyl)-1-methyl-4,6-dihydro-1H-pyrrolo[2,3-c]pyrazol-5-one
49 Acetic acid 4-[4-(4-acetoxy-phenyl)-1-methyl-5-oxo-1,4,5,6-tetrahydro-pyrrolo[2,3-c]pyrazol-4-yl]-phenyl ester
50 4,4-Bis-(4-amino-phenyl)-1-methyl-4,6-dihydro-1H-pyrrolo[2,3-c]pyrazol-5-one
51 N-{4-[4-(4-Methanesulfonylamino-phenyl)-1-methyl-5-oxo-1,4,5,6-tetrahydro-pyrrolo[2,3-c]pyrazol-4-yl]-phenyl}-methanesulfonamide
52 4,4-Bis-(4-dimethylamino-phenyl)-1-methyl-4,6-dihydro-1H-pyrrolo[2,3-c]pyrazol-5-one
53 N-{4-[4-(4-Acetylamino-phenyl)-1-methyl-5-oxo-1,4,5,6-tetrahydro-pyrrolo[2,3-c]pyrazol-4-yl]-phenyl}-acetamide
54 4,4-Bis-(4-hydroxy-phenyl)-2-methyl-2,6-dihydro-4H-pyrrolo[2,3-c]pyrazol-5-one
55 Acetic acid 4-[4-(4-acetoxy-phenyl)-2-methyl-5-oxo-2,4,5,6-tetrahydro-pyrrolo[2,3-c]pyrazol-4-yl]-phenyl ester
56 4,4-Bis-(4-amino-phenyl)-2-methyl-2,6-dihydro-4H-pyrrolo[2,3-c]pyrazol-5-one
57 4,4-Bis-(4-dimethylamino-phenyl)-2-methyl-2,6-dihydro-4H-pyrrolo[2,3-c]pyrazol-5-one
58 N-{4-[4-(4-Acetylamino-phenyl)-2-methyl-5-oxo-2,4,5,6-tetrahydro-pyrrolo[2,3-c]pyrazol-4-yl]-phenyl}-acetamide
59 N-{4-[4-(4-Methanesulfonylamino-phenyl)-2-methyl-5-oxo-2,4,5,6-tetrahydro-pyrrolo[2,3-c]pyrazol-4-yl]-phenyl}-methanesulfonamide
60 4,4-Bis-(4-hydroxy-phenyl)-4,6-dihydro-thieno[2,3-b]pyrrol-5-one
61 Acetic acid 4-[4-(4-acetoxy-phenyl)-5-oxo-5,6-dihydro-4H-thieno[2,3-b]pyrrol-4-yl]-phenyl ester
62 4,4-Bis-(4-amino-phenyl)-4,6-dihydro-thieno[2,3-b]pyrrol-5-one
63 4,4-Bis-(4-dimethylamino-phenyl)-4,6-dihydro-thieno[2,3-b]pyrrol-5-one
64 N-{4-[4-(4-Acetylamino-phenyl)-5-oxo-5,6-dihydro-4H-thieno[2,3-b]pyrrol-4-yl]-phenyl}-acetamide
65 N-{4-[4-(4-Methanesulfonylamino-phenyl)-5-oxo-5,6-dihydro-4H-thieno[2,3-b]pyrrol-4-yl]-phenyl}-methanesulfonamide
66 2-Chloro-4,4-bis-(4-hydroxy-phenyl)-4,6-dihydro-thieno[2,3-b]pyrrol-5-one
67 Acetic acid 4-[4-(4-acetoxy-phenyl)-2-chloro-5-oxo-5,6-dihydro-4H-thieno[2,3-b]pyrrol-4-yl]-phenyl ester
68 4,4-Bis-(4-amino-phenyl)-2-chloro-4,6-dihydro-thieno[2,3-b]pyrrol-5-one
69 2-Chloro-4,4-bis-(4-dimethylamino-phenyl)-4,6-dihydro-thieno[2,3-b]pyrrol-5-one
70 N-{4-[4-(4-Acetylamino-phenyl)-2-chloro-5-oxo-5,6-dihydro-4H-thieno[2,3-b]pyrrol-4-yl]-phenyl}-acetamide
71 N-{4-[2-Chloro-4-(4-methanesulfonylamino-phenyl)-5-oxo-5,6-dihydro-4H-thieno[2,3-b]pyrrol-4-yl]-phenyl}-methanesulfonamide
72 4,4-Bis-(4-hydroxy-phenyl)-4,6-dihydro-furo[2,3-b]pyrrol-5-one
73 Acetic acid 4-[4-(4-acetoxy-phenyl)-5-oxo-5,6-dihydro-4H-furo[2,3-b]pyrrol-4-yl]-phenyl ester
74 4,4-Bis-(4-amino-phenyl)-4,6-dihydro-furo[2,3-b]pyrrol-5-one
75 4,4-Bis-(4-dimethylamino-phenyl)-4,6-dihydro-furo[2,3-b]pyrrol-5-one
76 N-{4-[4-(4-Acetylamino-phenyl)-5-oxo-5,6-dihydro-4H-furo[2,3-b]pyrrol-4-yl]-phenyl}-acetamide
77 N-{4-[4-(4-Methanesulfonylamino-phenyl)-5-oxo-5,6-dihydro-4H-furo[2,3-b]pyrrol-4-yl]-phenyl}-methanesulfonamide
78 2-Chloro-4,4-bis-(4-hydroxy-phenyl)-4,6-dihydro-furo[2,3-b]pyrrol-5-one
79 Acetic acid 4-[4-(4-acetoxy-phenyl)-2-chloro-5-oxo-5,6-dihydro-4H-furo[2,3-b]pyrrol-4-yl]-phenyl ester
80 4,4-Bis-(4-amino-phenyl)-2-chloro-4,6-dihydro-furo[2,3-b]pyrrol-5-one
81 2-Chloro-4,4-bis-(4-dimethylamino-phenyl)-4,6-dihydro-furo[2,3-b]pyrrol-5-one
82 N-{4-[4-(4-Acetylamino-phenyl)-2-chloro-5-oxo-5,6-dihydro-4H-furo[2,3-b]pyrrol-4-yl]-phenyl}-acetamide
83 N-{4-[2-Chloro-4-(4-methanesulfonylamino-phenyl)-5-oxo-5,6-dihydro-4H-furo[2,3-b]pyrrol-4-yl]-phenyl}-methanesulfonamide
84 3,3-Bis-(4-hydroxy-phenyl)-6-methyl-3,8-dihydro-1H-1,8-diaza-as-indacen-2-one
85 Acetic acid 4-[3-(4-acetoxy-phenyl)-6-methyl-2-oxo-1,2,3,8-tetrahydro-1,8-diaza-as-indacen-3-yl]-phenyl ester
86 3,3-Bis-(4-amino-phenyl)-6-methyl-3,8-dihydro-1H-1,8-diaza-as-indacen-2-one
87 3,3-Bis-(4-dimethylamino-phenyl)-6-methyl-3,8-dihydro-1H-1,8-diaza-as-indacen-2-one
88 N-{4-[3-(4-Acetylamino-phenyl)-6-methyl-2-oxo-1,2,3,8-tetrahydro-1,8-diaza-as-indacen-3-yl]-phenyl}-acetamide
89 N-{4-[3-(4-Methanesulfonylamino-phenyl)-6-methyl-2-oxo-1,2,3,8-tetrahydro-1,8-diaza-as-indacen-3-yl]-phenyl}-methanesulfonamide
90 3,3-Bis-(4-hydroxy-phenyl)-1,3-dihydro-benzo[g]indol-2-one
91 Acetic acid 4-[3-(4-acetoxy-phenyl)-2-oxo-2,3-dihydro-1H-benzo[g]indol-3-yl]-phenyl ester
92 3,3-Bis-(4-amino-phenyl)-1,3-dihydro-benzo[g]indol-2-one
93 3,3-Bis-(4-dimethylamino-phenyl)-1,3-dihydro-benzo[g]indol-2-one
94 N-{4-[3-(4-Acetylamino-phenyl)-2-oxo-2,3-dihydro-1H-benzo[g]indol-3-yl]-phenyl}-acetamide
95 N-{4-[3-(4-Methanesulfonylamino-phenyl)-2-oxo-2,3-dihydro-1H-benzo[g]indol-3-yl]-phenyl}-methanesulfonamide
96 1-Amino-6-chloro-3,3-bis-(4-hydroxy-phenyl)-7-methyl-1,3-dihydro-indol-2-one
97 Acetic acid 4-[3-(4-acetoxy-phenyl)-1-amino-6-chloro-7-methyl-2-oxo-2,3-dihydro-1H-indol-3-yl]-phenyl ester
98 N-{4-[3-(4-Acetylamino-phenyl)-1-amino-6-chloro-7-methyl-2-oxo-2,3-dihydro-1H-indol-3-yl]-phenyl}-acetamide
99 N-{4-[1-Amino-6-chloro-3-(4-methanesulfonylamino-phenyl)-7-methyl-2-oxo-2,3-dihydro-1H-indol-3-yl]-phenyl}-methanesulfonamide
100 Acetic acid 4-[3-(4-acetoxy-phenyl)-1-acetylamino-6-chloro-7-methyl-2-oxo-2,3-dihydro-1H-indol-3-yl]-phenyl ester
101 N-[3,3-Bis-(4-amino-phenyl)-6-chloro-7-methyl-2-oxo-2,3-dihydro-indol-1-yl]-acetamide
102 N-[6-Chloro-3,3-bis-(4-dimethylamino-phenyl)-7-methyl-2-oxo-2,3-dihydro-indol-1-yl]-acetamide
103 N-[3,3-Bis-(4-acetylamino-phenyl)-6-chloro-7-methyl-2-oxo-2,3-dihydro-indol-1-yl]-acetamide
104 N-[6-Chloro-3,3-bis-(4-methanesulfonylamino-phenyl)-7-methyl-2-oxo-2,3-dihydro-indol-1-yl]-acetamide
105 6-Chloro-3,3-bis-(4-hydroxy-phenyl)-7-methyl-1,3-dihydro-indole-2-thione
106 Acetic acid 4-[3-(4-acetoxy-phenyl)-6-chloro-7-methyl-2-thioxo-2,3-dihydro-1H-indol-3-yl]-phenyl ester
107 3,3-Bis-(4-amino-phenyl)-6-chloro-7-methyl-1,3-dihydro-indole-2-thione
108 6-Chloro-3,3-bis-(4-dimethylamino-phenyl)-7-methyl-1,3-dihydro-indole-2-thione
109 N-{4-[3-(4-Acetylamino-phenyl)-6-chloro-7-methyl-2-thioxo-2,3-dihydro-1H-indol-3-yl]-phenyl}-acetamide
110 Methanesulfonic acid 4-[6-chloro-3-(4-methanesulfonyloxy-phenyl)-7-methyl-2-thioxo-2,3-dihydro-1H-indol-3-yl]-phenyl ester
111 Acetic acid 4-[4-(4-acetoxy-phenyl)-2-chloro-5-thioxo-5,6-dihydro-4H-thieno[2,3-b]pyrrol-4-yl]-phenyl ester
112 Acetic acid 4-[4-(4-acetoxy-phenyl)-2-chloro-5-thioxo-5,6-dihydro-4H-furo[2,3-b]pyrrol-4-yl]-phenyl ester
113 6,6-Bis-(4-amino-phenyl)-2-chloro-3-methyl-4,6-dihydro-thieno[3,2-b]pyrrole-5-thione
114 2-Chloro-6,6-bis-(4-dimethylamino-phenyl)-3-methyl-4,6-dihydro-3H-pyrrolo[2,3-d]imidazole-5-thione
115 N-{4-[6-(4-Acetylamino-phenyl)-3-chloro-5-thioxo-1,4,5,6-tetrahydro-pyrrolo[3,2-c]pyrazol-6-yl]-phenyl}-acetamide
116 Methanesulfonic acid 4-[2-chloro-4-(4-methanesulfonyloxy-phenyl)-5-thioxo-5,6-dihydro-4H-furo[2,3-b]pyrrol-4-yl]-phenyl ester
117 6-Chloro-7-cyclopropyl-3,3-bis-(4-hydroxy-phenyl)-1,3-dihydro-indol-2-one
118 6-Chloro-7-cyclopropyl-3,3-bis-(4-hydroxy-phenyl)-1,3-dihydro-pyrrolo[3,2-c]pyridin-2-one
119 6-Chloro-3,3-bis-(4-hydroxy-phenyl)-7-trifluoromethyl-1,3-dihydro-indol-2-one
120 6-Chloro-3,3-bis-(4-hydroxy-phenyl)-7-trifluoromethyl-1,3-dihydro-pyrrolo[3,2-c]pyridin-2-one
121 6-Chloro-7-cyclopropoxy-3,3-bis-(4-hydroxy-phenyl)-1,3-dihydro-indol-2-one
122 6-Chloro-7-cyclopropoxy-3,3-bis-(4-hydroxy-phenyl)-1,3-dihydro-pyrrolo[3,2-c]pyridin-2-one
123 6-(4-Fluoro-phenoxy)-3,3-bis-(4-hydroxy-phenyl)-7-trifluoromethyl-1,3-dihydro-indol-2-one
124 Acetic acid 4-[3-(4-acetoxy-phenyl)-6-chloro-7-cyclopropyl-2-oxo-2,3-dihydro-1H-indol-3-yl]-phenyl ester
125 Acetic acid 4-[3-(4-acetoxy-phenyl)-6-chloro-7-cyclopropyl-2-oxo-2,3-dihydro-1H-pyrrolo[3,2-c]pyridin-3-yl]-phenyl ester
126 Acetic acid 4-[3-(4-acetoxy-phenyl)-6-chloro-2-oxo-7-trifluoromethyl-2,3-dihydro-1H-indol-3-yl]-phenyl ester
127 Acetic acid 4-[3-(4-acetoxy-phenyl)-6-chloro-2-oxo-7-trifluoromethyl-2,3-dihydro-1H-pyrrolo[3,2-c]pyridin-3-yl]-phenyl ester
128 Acetic acid 4-[3-(4-acetoxy-phenyl)-6-chloro-7-cyclopropoxy-2-oxo-2,3-dihydro-1H-indol-3-yl]-phenyl ester
129 Acetic acid 4-[3-(4-acetoxy-phenyl)-6-chloro-7-cyclopropoxy-2-oxo-2,3-dihydro-1H-pyrrolo[3,2-c]pyridin-3-yl]-phenyl ester
130 Acetic acid 4-[3-(4-acetoxy-phenyl)-6-(4-fluoro-phenoxy)-2-oxo-7-trifluoromethyl-2,3-dihydro-1H-indol-3-yl]-phenyl ester
131 Dimethylamino-acetic acid 4-{6-chloro-7-cyclopropyl-3-[4-(2-dimethylamino-acetoxy)-phenyl]-2-oxo-2,3-dihydro-1H-indol-3-yl}-phenyl ester
132 Dimethylamino-acetic acid 4-{6-chloro-7-cyclopropyl-3-[4-(2-dimethylamino-acetoxy)-phenyl]-2-oxo-2,3-dihydro-1H-pyrrolo[3,2-c]pyridin-3-yl}-phenyl ester
133 Dimethylamino-acetic acid 4-{6-chloro-3-[4-(2-dimethylamino-acetoxy)-phenyl]-7-methyl-2-oxo-2,3-dihydro-1H-indol-3-yl}-phenyl ester
134 6-Chloro-3,3-bis-(4-hydroxy-phenyl)-7-trifluoromethoxy-1,3-dihydro-indol-2-one
135 Acetic acid 4-[3-(4-acetoxy-phenyl)-6-chloro-2-oxo-7-trifluoromethoxy-2,3-dihydro-1H-indol-3-yl]-phenyl ester
136 Dimethylamino-acetic acid 4-{6-chloro-3-[4-(2-dimethylamino-acetoxy)-phenyl]-2-oxo-7-trifluoromethoxy-2,3-dihydro-1H-indol-3-yl}-phenyl ester
137 6-Chloro-4-fluoro-3,3-bis-(4-hydroxy-phenyl)-7-methyl-1,3-dihydro-indol-2-one
138 3-Chloro-7,7-bis-(4-hydroxy-phenyl)-4-methyl-5,7-dihydro-pyrrolo[3,2-c]pyridazin-6-one
139 Acetic acid 4-[3-(4-acetoxy-phenyl)-6-chloro-4-fluoro-7-methyl-2-oxo-2,3-dihydro-1H-indol-3-yl]-phenyl ester
140 Acetic acid 4-[3-(4-acetoxy-phenyl)-6-chloro-4,7-dimethyl-2-oxo-2,3-dihydro-1H-indol-3-yl]-phenyl ester
141 Acetic acid 4-[7-(4-acetoxy-phenyl)-3-chloro-4-methyl-6-oxo-6,7-dihydro-5H-pyrrolo[3,2-c]pyridazin-7-yl]-phenyl ester
142 6-Chloro-4,5-difluoro-3,3-bis-(4-hydroxy-phenyl)-7-methyl-1,3-dihydro-indol-2-one
143 Acetic acid 4-[3-(4-acetoxy-phenyl)-6-chloro-4,5-difluoro-7-methyl-2-oxo-2,3-dihydro-1H-indol-3-yl]-phenyl ester
144 3,3-Bis-(4-hydroxy-phenyl)-3,6,7,8-tetrahydro-1H-1-aza-as-indacen-2-one
145 3,3-Bis-(4-hydroxy-phenyl)-1,3,6,7,8,9-hexahydro-benzo[g]indol-2-one
146 3,3-Bis-(4-hydroxy-phenyl)-7-trifluoromethyl-1,3-dihydro-indol-2-one
147 7-Chloro-3,3-bis-(4-hydroxy-phenyl)-1,3-dihydro-indol-2-one
148 3,3-Bis-(4-hydroxy-phenyl)-2-oxo-2,3-dihydro-1H-indole-7-carbonitrile
149 7-Ethyl-3,3-bis-(4-hydroxy-phenyl)-1,3-dihydro-indol-2-one
150 3,3-Bis-(4-hydroxy-phenyl)-7-morpholin-4-yl-1,3-dihydro-indol-2-one
151 3,3-Bis-(4-hydroxy-phenyl)-7-isopropyl-1,3-dihydro-indol-2-one
152 7-tert-Butyl-3,3-bis-(4-hydroxy-phenyl)-1,3-dihydro-indol-2-one
153 3,3-Bis-(4-hydroxy-phenyl)-2-oxo-2,3-dihydro-1H-indole-7-carboxylic acid dimethylamide
154 3,3-Bis-(4-hydroxy-phenyl)-7-(4-methyl-piperazine-1-carbonyl)-1,3-dihydro-indol-2-one
155 3,3-Bis-(4-hydroxy-phenyl)-2-oxo-2,3-dihydro-1H-indole-5-carboxylic acid
156 3,3-Bis-(4-hydroxy-phenyl)-2-oxo-2,3-dihydro-1H-indole-5-carboxylic acid dimethylamide
157 3,3-Bis-(4-hydroxy-phenyl)-5-(morpholine-4-carbonyl)-1,3-dihydro-indol-2-one
158 3,3-Bis-(4-hydroxy-phenyl)-4-methoxy-1,3-dihydro-indol-2-one
159 3,3-Bis-(4-hydroxy-phenyl)-6-methoxy-1,3-dihydro-indol-2-one
160 3,3-Bis-(4-hydroxy-phenyl)-5-(4-methyl-piperazine-1-carbonyl)-1,3-dihydro-indol-2-one
161 6-Chloro-3,3-bis-(4-mercapto-phenyl)-7-methyl-1,3-dihydro-indol-2-one
162 N-{4-[3-(4-Acetylamino-phenyl)-7-methyl-2-oxo-2,3-dihydro-1H-indol-3-yl]-phenyl}-acetamide
163 3,3-Bis-(4-hydroxy-phenyl)-7-(3-methoxy-prop-1-ynyl)-1,3-dihydro-indol-2-one
164 3,3-Bis-(4-hydroxy-phenyl)-7-pyridin-3-yl-1,3-dihydro-indol-2-one
165 7-Bromo-3,3-bis-(4-hydroxy-phenyl)-1,3-dihydro-indol-2-one
166 6-Chloro-3,3-bis-(4-methanesulfonyl-phenyl)-7-methyl-1,3-dihydro-indol-2-one
167 6,6-Bis-(4-hydroxy-phenyl)-4,6-dihydro-pyrrolo[3,2-d]thiazol-5-one
168 6,6-Bis-(4-hydroxy-phenyl)-2-methyl-4,6-dihydro-pyrrolo[3,2-d]thiazol-5-one
169 6,6-Bis-(4-hydroxy-phenyl)-2-isopropyl-4,6-dihydro-pyrrolo[3,2-d]thiazol-5-one
170 2-Chloro-6,6-bis-(4-hydroxy-phenyl)-4,6-dihydro-pyrrolo[3,2-d]thiazol-5-one
171 4,4-Bis-(4-hydroxy-phenyl)-4,6-dihydro-pyrrolo[3,2-d]isothiazol-5-one
172 3,3-Bis-(4-hydroxy-phenyl)-7-methyl-1,3-dihydro-pyrrolo[2,3-c]pyridin-2-one
173 3,3-Bis-(4-hydroxy-phenyl)-7-methyl-1,3-dihydro-pyrrolo[3,2-b]pyridin-2-one
174 3,3-Bis-(4-fluoro-phenyl)-7-methyl-1,3-dihydro-pyrrolo[3,2-b]pyridin-2-one
175 3,3-Bis-(4-fluoro-phenyl)-7-methyl-1,3-dihydro-pyrrolo[3,2-c]pyridin-2-one
176 3,3-Bis-(4-fluoro-phenyl)-7-isopropyl-1,3-dihydro-pyrrolo[3,2-c]pyridin-2-one
177 3,3-Bis-(4-hydroxy-phenyl)-3,6,7,8-tetrahydro-1H-1,5-diaza-as-indacen-2-one
178 3,3-Bis-(4-hydroxy-phenyl)-3,6,7,8-tetrahydro-1H-1,4-diaza-as-indacen-2-one
179 3,3-Bis-(4-hydroxy-phenyl)-1,3,6,7,8,9-hexahydro-pyrrolo[3,2-c]quinolin-2-one
180 3,3-Bis-(4-hydroxy-phenyl)-1,3,6,7,8,9-hexahydro-pyrrolo[3,2-c]isoquinolin-2-one
181 5-Fluoro-3,3-bis-(4-hydroxy-phenyl)-3,6,7,8-tetrahydro-1H-1-aza-as-indacen-2-one
182 7-Ethyl-5-fluoro-3,3-bis-(4-hydroxy-phenyl)-1,3-dihydro-indol-2-one
183 3,3-Bis-(4-hydroxy-phenyl)-1,3,6,8-tetrahydro-7-oxa-1-aza-as-indacen-2-one
184 3,3-Bis-(4-hydroxy-phenyl)-1,3,7,8-tetrahydro-6-oxa-1-aza-as-indacen-2-one
185 3,3-Bis-(4-hydroxy-phenyl)-1,6,7,9-tetrahydro-3H-8-oxa-1-aza-cyclopenta[a]naphthalen-2-one
186 3,3-Bis-(4-hydroxy-phenyl)-1,7,8,9-tetrahydro-3H-pyrano[2,3-g]indol-2-one
187 3,3-Bis-(4-hydroxy-phenyl)-7-methyl-3,6,7,8-tetrahydro-1H-1,7-diaza-as-indacen-2-one
188 3,3-Bis-(4-hydroxy-phenyl)-7-methyl-1,3,7,8-tetrahydro-1,7-diaza-as-indacene-2,6-dione
189 3,3-Bis-(4-hydroxy-phenyl)-7,8,8-trimethyl-1,3,7,8-tetrahydro-1,7-diaza-as-indacene-2,6-dione
190 3,3-Bis-(4-hydroxy-phenyl)-5-iodo-1,3-dihydro-indol-2-one
191 5-Amino-3,3-bis-(4-hydroxy-phenyl)-1,3-dihydro-indol-2-one
192 5-Amino-3,3-bis-(4-hydroxy-phenyl)-7-methyl-1,3-dihydro-indol-2-one
193 6-Bromo-3,3-bis-(4-hydroxy-phenyl)-7-methyl-1,3-dihydro-indol-2-one
194 7-Fluoro-3,3-bis-(4-hydroxy-phenyl)-1,3-dihydro-indol-2-one
195 3,3-Bis-(4-hydroxy-phenyl)-7-methoxy-1,3-dihydro-indol-2-one
196 4,7-Dichloro-3,3-bis-(4-hydroxy-phenyl)-1,3-dihydro-indol-2-one
197 6-Chloro-3,3-bis-(4-hydroxy-phenyl)-1,7-dimethyl-1,3-dihydro-indol-2-one
198 6-Chloro-3,3-bis-(4-fluoro-phenyl)-7-methyl-1,3-dihydro-indol-2-one
199 3,3-Bis-(4-hydroxy-phenyl)-7-(morpholine-4-carbonyl)-1,3-dihydro-indol-2-one
200 3,3-Bis-(4-hydroxy-phenyl)-1,3-dihydro-pyrrolo[2,3-d]pyridin-2-one
201 N-{4-[6-Chloro-3-(4-methanesulfonylamino-phenyl)-7-methyl-2-oxo-2,3-dihydro-1H-indol-3-yl]-phenyl}-methanesulfonamide
202 3,3-Bis-(4-hydroxy-phenyl)-4,7-dimethyl-1,3-dihydro-indol-2-one
203 3,3-Bis-(4-hydroxy-phenyl)-7-iodo-1,3-dihydro-indol-2-one
204 3,3-Bis-(4-hydroxy-phenyl)-7-pyridin-4-yl-1,3-dihydro-indol-2-one
205 Acetic acid 4-[3-(4-acetoxy-phenyl)-6-chloro-7-methyl-2-oxo-2,3-dihydro-1H-indol-3-yl]-phenyl ester
206 3,3-Bis-(4-hydroxy-phenyl)-5-phenyl-1,3-dihydro-indol-2-one
207 3,3-Bis-(4-hydroxy-phenyl)-7-thiophen-2-yl-1,3-dihydro-indol-2-one
208 3,3-Bis-(4-hydroxy-phenyl)-5-pyridin-4-yl-1,3-dihydro-indol-2-one
209 3,3-Bis-(4-hydroxy-phenyl)-5-thiophen-2-yl-1,3-dihydro-indol-2-one
210 5,7-Difluoro-3,3-bis-(4-hydroxy-phenyl)-1,3-dihydro-indol-2-one
211 6-Fluoro-3,3-bis-(4-hydroxy-phenyl)-7-methyl-1,3-dihydro-indol-2-one
212 3,3-Bis-(4-hydroxy-phenyl)-6-methoxy-7-methyl-1,3-dihydro-indol-2-one
213 6,7-Difluoro-3,3-bis-(4-hydroxy-phenyl)-1,3-dihydro-indol-2-one
214 6-Chloro-7-fluoro-3,3-bis-(4-hydroxy-phenyl)-1,3-dihydro-indol-2-one
215 5-Fluoro-3,3-bis-(4-hydroxy-phenyl)-7-methyl-1,3-dihydro-indol-2-one
216 3,3-Bis-(4-hydroxy-phenyl)-5-methoxy-7-methyl-1,3-dihydro-indol-2-one
217 3,3-Bis-(4-hydroxy-phenyl)-1,3-dihydro-pyrrolo[2,3-b]pyridin-2-one
218 7-Chloro-3,3-bis-(4-hydroxy-phenyl)-4-methoxy-1,3-dihydro-indol-2-one
219 6-Fluoro-3,3-bis-(4-hydroxy-phenyl)-1,3-dihydro-indol-2-one
220 N-[3,3-Bis-(4-hydroxy-phenyl)-2-oxo-2,3-dihydro-indol-1-yl]-acetamide
221 5-[3,3-Bis-(4-hydroxy-phenyl)-7-methyl-2-oxo-2,3-dihydro-1H-indol-6-yloxy]-pentanoic acid methyl ester
222 5-[3,3-Bis-(4-hydroxy-phenyl)-7-methyl-2-oxo-2,3-dihydro-1H-indol-6-yloxy]-pentanoic acid
223 5-[3,3-Bis-(4-hydroxy-phenyl)-7-methyl-2-oxo-2,3-dihydro-1H-indol-5-yloxy]-pentanoic acid methyl ester
224 5-[3,3-Bis-(4-hydroxy-phenyl)-7-methyl-2-oxo-2,3-dihydro-1H-indol-5-yloxy]-pentanoic acid
225 7-Chloro-6-fluoro-3,3-bis-(4-hydroxy-phenyl)-1,3-dihydro-indol-2-one.
   Method of Treatment

A further aspect of the present invention relates to a method of treating a mammal suffering from or being susceptible to cancer, the method comprising administering to the mammal a therapeutically effective amount of a compound defined hereinabove. Conditions with respect to dosage, administration, etc. may be as defined further below.

Biological Effects

The present inventors have found that many compounds of general formula (I) are shown to inhibit the proliferation of MDA-468 cells at lower concentrations as those required to inhibit proliferation of MDA-231 cells. A possible mechanism to explain this finding is the selective inhibition of protein synthesis by compounds of general formula (I) in MDA-468 cells compared to MDA-231 cells. Our present hypothesis is that compounds of the general formula (I) inhibit protein synthesis by selective inhibition of mTOR pathway activation and/or other biochemical pathways involved in the regulation of protein synthesis.

The selective inhibition of mTOR pathway activation by compounds of the general formula (I) in Western blots correlates with cell proliferation and protein synthesis data. This suggests that detection of mTOR pathway activity by measurement of either p70S6K, 4E-BP1 or S6K phosphorylation status using phosphor-specific or total protein antibodies by Western blot or ELISA, or measurement of p70S6K kinase activity, in patient tumour material or blood samples, may provide a useful method for selecting patients who will respond to compounds of general formula (I). Alternatively, measurement of p70S6K or S6K phosphorylation status using phosphospecific antibodies, or p70S6K kinase activity, in tumour material or blood samples may provide a biomarker useful for determining drug dosing of compounds of the general formula (I) in human clinical trials.

Compounds for Medical Use

Apart from the more specific medical use outlined above, it is also believed that the majority of the compounds defined herein are generally applicable for medical use.

Thus, in a further aspect the present invention relates to a compound as defined hereinabove for use as a medicament, with the proviso that the compound is not one selected from 3,3-bis-(4-hydroxy-phenyl)-1,3-dihydro-indol-2-one and acetic acid 4-[3-(4-acetoxy-phenyl)-2-oxo-2,3-dihydro-1H-indol-3-yl]-phenyl ester. Particularly interesting compounds of the Formula (I) are those of the formulae (IIa), (IIb), (IIc) and (IId) defined above.

Novel Compounds

As mentioned in the introductory section, a few compounds according to the general formula (I) have been described in the literature and (unrelated) biological effects have previously been described for some of these compounds.

Thus, a still further aspect of the present invention relates to a compound of the formula (I) 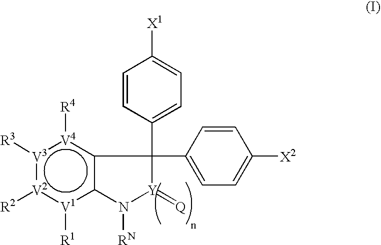
as defined further above, with the proviso that the compound is not one selected from
3,3-bis,-(4-hydroxy-phenyl)-1,3-dihydro-indol-2-one,
3,3-bis-(4-hydroxy-phenyl)-7-methyl-1,3-dihydro-indol-2-one;
3,3-bis-(4-hydroxy-phenyl)-4,5-dimethyl-1,3-dihydro-indol-2-one
3,3-bis-(4-hydroxy-phenyl)-5,7-dimethyl-1,3-dihydro-indol-2-one;
5-bromo-3,3-bis-(4-hydroxy-phenyl)-1,3-dihydro-indol-2-one;
5-chloro-3,3-bis-(4-hydroxy-phenyl)-1,3-dihydro-indol-2-one;
3,3-bis-(4-hydroxy-phenyl)-5-methoxy-1,3-dihydro-indol-2-one;
3,3-bis-(4-hydroxy-phenyl)-5-methyl-1,3-dihydro-indol-2-one;
6-chloro-3,3-bis-(4-hydroxy-phenyl)-7-methyl-1,3-dihydro-indol-2-one;
acetic acid 4-[3-(4-acetoxy-phenyl)-2-oxo-2,3-dihydro-1H-indol-3-yl]-phenyl ester; and
acetic acid 4-[3-(4-acetoxy-phenyl)-5-methyl-2-oxo-2,3-dihydro-1H-indol-3-yl]-phenyl ester.

The specification of the compound of the formula (I) and the preferences are as described hereinabove. In particular, preferred compounds of the Formula (I) are those of the formulae (IIa), (IIb), (IIc) and (IId) defined above.

Preparation of Compounds of the Formula (I) and the Formula (IIa)-(IId)

The compounds generally can be synthesized as described in the Examples section.

Formulation of Pharmaceutical Compositions

The compound of the formula (I) (and the more specific compound of the formula (II)) is suitably formulated in a pharmaceutical composition so as to suit the desirable route of administration.

The administration route of the compounds may be any suitable route which leads to a concentration in the blood or tissue corresponding to a therapeutic effective concentration. Thus, e.g., the following administration routes may be applicable although the invention is not limited thereto: the oral route, the parenteral route, the cutaneous route, the nasal route, the rectal route, the vaginal route and the ocular route. It should be clear to a person skilled in the art that the administration route is dependent on the particular compound in question; particularly the choice of administration route depends on the physico-chemical properties of the compound together with the age and weight of the patient and on the particular disease or condition and the severity of the same.

The compounds may be contained in any appropriate amount in a pharmaceutical composition, and are generally contained in an amount of about 1-95%, e.g. 1-10%, by weight of the total weight of the composition. The composition may be presented in a dosage form which is suitable for the oral, parenteral, rectal, cutaneous, nasal, vaginal and/or ocular administration route. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, aerosols and in other suitable form.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988. Typically, the compounds defined herein are formulated with (at least) a pharmaceutically acceptable carrier or excipient. Pharmaceutically acceptable carriers or excipients are those known by the person skilled in the art. Formation of suitable salts of the compounds of the Formula I will also be evident in view of the before-mentioned.

Thus, the present invention provides in a further aspect a pharmaceutical composition comprising a compound of the general Formula I in combination with a pharmaceutically acceptable carrier.

The compound is preferably one of those defined under “Compounds for medical use”.

In a particular embodiment, the compound is as defined under “Novel compounds”, i.e. novel compounds of the Formula (I) and Formula (II) respectively.

Pharmaceutical compositions according to the present invention may be formulated to release the active compound substantially immediately upon administration or at any substantially predetermined time or time period after administration. The latter type of compositions is generally known as controlled release formulations.

In the present context, the term “controlled release formulation” embraces i) formulations which create a substantially constant concentration of the drug within the body over an extended period of time, ii) formulations which after a predetermined lag time create a substantially constant concentration of the drug within the body over an extended period of time, iii) formulations which sustain drug action during a predetermined time period by maintaining a relatively, constant, effective drug level in the body with concomitant minimization of undesirable side effects associated with fluctuations in the plasma level of the active drug substance (sawtooth kinetic pattern), iv) formulations which attempt to localize drug action by, e.g., spatial placement of a controlled release composition adjacent to or in the diseased tissue or organ, v) formulations which attempt to target drug action by using carriers or chemical derivatives to deliver the drug to a particular target cell type.

Controlled release formulations may also be denoted “sustained release”, “prolonged release”, “programmed release”, “time release”, “rate-controlled” and/or “targeted release” formulations.

Controlled release pharmaceutical compositions may be presented in any suitable dosage forms, especially in dosage forms intended for oral, parenteral, cutaneous nasal, rectal, vaginal and/or ocular administration. Examples include single or multiple unit tablet or capsule compositions, oil solutions, suspensions, emulsions, microcapsules, microspheres, nanoparticles, liposomes, delivery devices such as those intended for oral, parenteral, cutaneous, nasal, vaginal or ocular use.

Preparation of solid dosage forms for oral use, controlled release oral dosage forms, fluid liquid compositions, parenteral compositions, controlled release parenteral compositions, rectal compositions, nasal compositions, percutaneous and topical compositions, controlled release percutaneous and topical compositions, and compositions for administration to the eye will be well-known to those skilled in the art of pharmaceutical formulation. Specific formulations can be found in “Remington's Pharmaceutical Sciences”.

Capsules, tablets and pills etc. may contain for example the following compounds: microcrystalline cellulose, gum or gelatin as binders; starch or lactose as excipients; stearates as lubricants; various sweetening or flavouring agents. For capsules the dosage unit may contain a liquid carrier like fatty oils. Likewise coatings of sugar or enteric agents may be part of the dosage unit. The pharmaceutical compositions may also be emulsions of the compound(s) and a lipid forming a micellular emulsion.

For parenteral, subcutaneous, intradermal or topical administration the pharmaceutical composition may include a sterile diluent, buffers, regulators of tonicity and antibacterials. The active compound may be prepared with carriers that protect against degradation or immediate elimination from the body, including implants or microcapsules with controlled release properties. For intravenous administration the preferred carriers are physiological saline or phosphate buffered saline.

Dosages

In one embodiment, the pharmaceutical composition is in unit dosage form. In such embodiments, each unit dosage form typically comprises 0.1-500 mg, such as 0.1-200 mg, e.g. 0.1-100 mg, of the compound.

More generally, the compound are preferably administered in an amount of about 0.1-250 mg per kg body weight per day, such as about 0.5-100 mg per kg body weight per day.

For compositions adapted for oral administration for systemic use, the dosage is normally 0.5 mg to 1 g per dose administered 1-4 times daily for 1 week to 12 months depending on the disease to be treated.

The dosage for oral administration of the composition in order to prevent diseases or conditions is normally 1 mg to 100 mg per kg body weight per day. The dosage may be administered once or twice daily for a period starting 1 week before the exposure to the disease until 4 weeks after the exposure.

For compositions adapted for rectal use for preventing diseases, a somewhat higher amount of the compound is usually preferred, i.e. from approximately 1 mg to 100 mg per kg body weight per day.

For parenteral administration, a dose of about 0.1 mg to about 100 mg per kg body weight per day is convenient. For intravenous administration, a dose of about 0.1 mg to about 20 mg per kg body weight per day administered for 1 day to 3 months is convenient. For intraarticular administration, a dose of about 0.1 mg to about 50 mg per kg body weight per day is usually preferable. For parenteral administration in general, a solution in an aqueous medium of 0.5-2% or more of the active ingredients may be employed.

For topical administration on the skin, a dose of about 1 mg to about 5 g administered 1-10 times daily for 1 week to 12 months is usually preferable.

Combination Treatment

In an intriguing embodiment of the present invention, the compound of the general formula (I) or the general formula (II) is used therapeutically in combination with one or more other chemotherapeutic agents. Examples of such chemotherapeutic agents are those selected from daunorubicin, docetaxel, prednisone, dexamethasone, decadron, altretamine, amifostine, aminoglutethimide, dactinomycin, anastrozole, asparaginase, bicalutamide, bleomycin, busulfan, carboplatin, carmustine, chlorambucil, chlorodeoxyadenosine, cisplatin, cytosine arabinoside, dacarbazine, doxorubicin, epirubicin, estramustine, diethylstilbestrol, fludarabine, flutamide, 5-fluorouracil, gemcitabine, goserelin, idarubicin, irinotecan, levamisole, lomustine, mechlorathamine, alkeran, mercaptopurine, taxol (e.g. paclitaxel). In particular, the further chemotherapeutic agent is selected from taxanes such as Taxol, Paclitaxel and Docetaxel.

Thus, with respect to the use and the method of treatment defined herein, the medicament may further comprise one or more other chemotherapeutic agents.

With respect to the pharmaceutical composition defined herein, such a composition may further comprise one or more other chemotherapeutic agents.

### EXAMPLES

### Materials

The following cell lines were obtained from ATCC: MDA-MB-231, MDA-MB-435S, MDA-MB-453, MDA-MB-468, SKBr-3, BT-474, BT-549, MCF-7, MCF10A, T-47D, ZR75-1, HCC-1954, DU-145, PC-3, LnCaP, and Colo205. PC-3/M was obtained from NCI. Terfenadine was obtained from Sigma-Aldrich. Penicillin-Streptomycin and gentamicin was purchased from Invitrogen. Alamar Blue reagent is from BioSource.

Starting materials, reagents and solvents for the chemical syntheses were obtained from commercial sources unless otherwise stated. Oxyphenisatine (Commercial A) and 7-methyl-oxyphenisatine (Commercial B) were also obtained from commercials sources.

### Example 1

### Procedures for Preparation of Isatin Derivatives

Isatin derivatives used as intermediates can be obtained by either Protocol A or Protocol B.

Protocol A, based on literature procedures, was used to generate aromatic isatins with either electron-donating substituents (see Stolle: J. Prakt. Chem. (1922), 105, 137 and Sandmeyer: Helv. Chim. Acta (1919), 2, 234) or a 5-membered electron rich heteroaromatic moiety (see Shvedov et al. (Chem. Heterocycl. Compd. Engl. Transl. (1975), 11, 666). Examples of preferred 5-membered heterocycles are thiophenes (V¹═S, V²═V³═C(−) and V⁴=bond; V²═S, V¹═V³═C(−) and V⁴=bond or V³═S, V¹═V²═C(−) and V⁴=bond), furans (V¹═O, V²═V³═C(−) and V⁴=bond; V²═O, V¹═V³═C(−) and V⁴=bond or V³═O, V¹═V²═C(−) and V⁴=bond), pyrazoles (V¹═N(−), V²═N, V³═C(−) and V⁴=bond; V¹═N, V²═N(−), V³═C(−) and V⁴=bond) and imidazoles (V¹═N(−), V²═C(−), V³═N and V⁴=bond; V¹═N, V²═C(−), V³═N(−) and V⁴=bond).

Protocol B, based on literature procedures, was used to generate aromatic isatins with electron-withdrawing substituents (see Hewawasam and Maenwell: Tet. Lett. (1994), 35, 7303) and 6-membered electron-poor heteroaromatic isatins (see Rivalle and Bisagni: J. Heterocycl. Chem. (1997), 34, 441). Examples of preferred 6-membered heterocycles are pyridines (V¹═N, V²═V³═V⁴═C(−); V²═N, V¹═V³═V⁴═C(−); V³═N, V¹═V²═V⁴═C(−) and V⁴═N, V¹═V²═V³═C(−)), pyrimidines (V¹═V³═N, V²═V⁴═C(−); V²═V⁴═N, V¹═V³═C(−)), pyrazines (V¹═V⁴═N, V²═V³═C(−)) and pyrimidines (V¹═V²═N, V³═V⁴═C(−); V²═V³═N, V¹═V⁴═C(−); V³═V⁴═N, V¹═V²═C(−)).

Other isatins of interest could in addition be prepared using one of the alternative methods published in the literature (see i.e. Tatsugi et al. ARKIVOC (2001), 67-73 or the review by Silva et al. in J. Braz. Chem. Soc. (2001), 12, 273-324).
Protocol A: Preparation of Isatin Derivatives

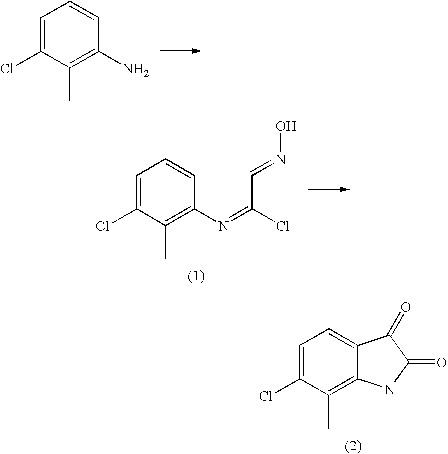

To a well stirred suspension of sodium sulfate (314 g, 2211 mmol) in water (700 mL) at 60° C. were added in sequence hydroxylamine hydrochloride (56 g, 806 mmol), chloral hydrate (47 g, 284 mmol), 2-methyl-3-chloro-aniline (40 g, 283 mmol) in water (500 mL) and finally concentrated hydrochloric acid (12 M, 24.2 ml, 290 mmol). The mixture temperature was risen to 100° C. After 20 minutes, the brown solution was left to cool to room temperature and kept stirring overnight. The solid present was filtered, washed with water (3×), heptane (2×) and dried at 60° C. under vacuum for 6 hours. Obtained 62 g of N-(3-chloro-2-methyl-phenyl)-2-hydroxyimino-acetimidoyl chloride (1) as a beige solid used without further purification. δ_{H} (400 MHz, DMSO-d6) 12.3 (1H, s), 9.8 (1H, s), 7.7 (1H, s), 7.42 (1H, d, J=7.8), 7.36 (1H, d, J=7.6), 7.3 (1H, m), 2.25 (3H, s).

To well stirred sulphuric acid (18.3 M, 300 ml) heated at 50° C. was added N-(3-chloro-2-methyl-phenyl)-2-hydroxyimino-acetimidoyl chloride (1) in small portion over 20 minutes (exothermic up to 70° C.) (60 g, 282 mmol). After addition was completed, the temperature was risen to 80° C. and kept for 20 minutes after which the reaction was left cool to room temperature. The brown mixture was slowly poured into ice (˜500 g) and water (500 mL), diluted with more water (1 L) to yield a brown-orange slurry. The solid was collected by filtration, washed with water (2×) under suction to yield an orange solid. This solid was dissolved in 0.4 M sodium hydroxide (1 L). All insoluble tar was removed by filtration. Concentrated hydrochloric acid (12 M, 70 mL) was added, the resulting brown-orange solid was collected by filtration, washed with water (3×), heptane (2×) and dried at 54° C. under vacuum for 6 hours. Obtained 34.5 g (208 mmol, 62%) of 6-chloro-7-methyl-1H-indole-2,3-dione (2). δ_{H} (400 MHz, DMSO-d6) 11.3 (1H, s), 7.4 (1H, d, J=8.0), 7.2 (1H, d, J=8.1), 2.25 (3H, s).
Protocol B: Preparation of Isatin Derivatives

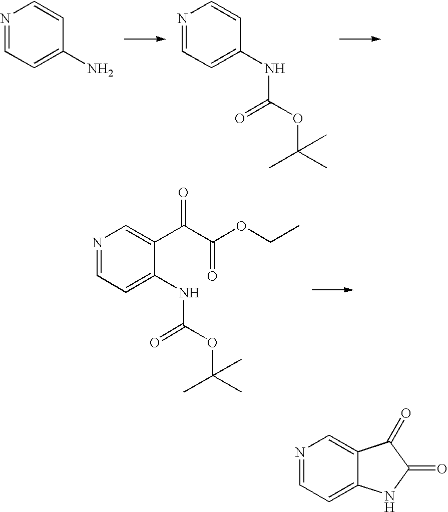

To a well stirred solution of Boc anhydride (2.56 g, 11.7 mmol) in THF (10 mL) was added 4-aminopyridine (1.0 g, 10.6 mmol) in portions over 3 minutes while maintaining the temperature between 20° C. and 25° C. No more exotherm was observed after 5 minutes. The reaction was then stirred at room temperature for 3.5 hours. After in vacuo concentration the crude mixture was then titurated in hexane (20 mL), filtered and washed with more hexane (˜5 mL). The resulting solid dried under reduced pressure to yield 1.93 g (9.9 mmol, 94%) of pyridin-4-yl-carbamic acid tert-butyl ester as a white solid and was used without further purification. LCMS (BDS-Hypersil C₁₈, 50 mm×2.1 mm, 5μ, 2.5 minutes) m/z 195 [MH]⁺ @ retention time 0.90 minutes, 100% by UV at 215 nm.

To a stirred solution of pyridin-4-yl-carbamic acid tert-butyl ester (0.62 g, 3.09 mmol) in THF (9 mL) cooled to −5° C. was slowly added a solution of t-BuLi (1.7M in THF, 5.5 mL, 9.27 mmol) over 17 minutes while maintaining the temperature between −5° C. and 1° C. A red brown precipitate resulted and the reaction mixture stirred at 0° C. for a further 1.5 hours. The reaction mixture was then cooled back down to −5° C. and diethyloxalate (1.3 mL, 9.27 mmol) was added. The reaction was allowed to reach room temperature and then after 2 hours quenched with water (10 mL). After in vacuo concentration the resulting mixture was diluted in ethyl acetate (20 mL) and washed with water (10 mL), dried over Na₂SO₄ and concentrated in vacuo. Purification by flash column chromatography (30% EtOAc/Hexane) afforded 0.16 g (0.54 mmol, 17%) of (4-tert-butoxycarbonylamino-pyridin-3-yl)-oxo-acetic acid ethyl ester as a brown oil.

LCMS (BDS-Hypersil C₁₈, 50 mm×2.1 mm, 5μ, 2.5 minutes) m/z 295 [MH]⁺+H₂O adduct @ retention time 1.07 minute, 96% by UV at 215 nm

(4-tert-Butoxycarbonylamino-pyridin-3-yl)-oxo-acetic acid ethyl ester (0.14 g, 0.476 mmol) was heated at 186° C. under 5 mmHg for 25 minutes in a Kugelrohr apparatus. The brown oil darkens and subsequently gives off gases to form a dark green solid. The solid was dissolved in MeOH and concentrated in vacuo to yield 0.04 g (0.3 mmol, 56%) of 1H-pyrrolo[3,2-c]pyridine-2,3-dione as a dark solid. The isatin was then taken to the next step without further purification.

Protocol C: Introduction of Functional Groups on the Isatin Derivatives

### 6-Chloro-7-methyl-5-nitro-1H-indole-2,3-dione (4)

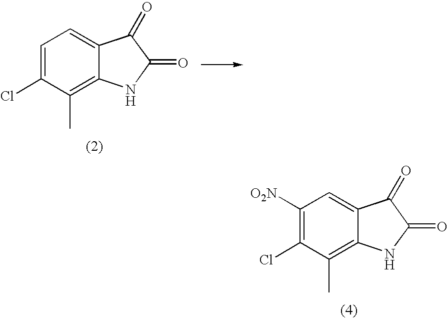

To a well stirred suspension of 2 (2.0 g, 10.2 mmol) in glacial acetic acid (2 mL) and sulphuric acid (4 mL) cooled in ice/water was added a cold mixture of nitric acid (69%, 1 g, 10.9 mmol) and sulphuric acid (0.7 g, 7.3 mmol) at such a rate to maintain internal temperature below 5° C. After addition was completed reaction mixture was stirred at room temperature for 1 h, then slowly poured over ice (˜20 g) and left standing for 10 minutes. The solid formed was collected by filtration, washed with cold water (3×), dried under vacuum overnight to yield 1.92 g (8.0 mmol, 78%) of 6-chloro-7-methyl-5-nitro-1H-indole-2,3-dione (4) as an orange solid. LCMS m/z 118.79 [Fragment]⁺ @ R_{T} 1.14 min, 95%

### Example 2

### Procedures for Preparation of the Final Compounds of the Invention

The obtained isatin derivatives were used to generate the final compounds of the invention. Typically, an isatin derivative was heated with a benzene derivative to 100° C. in a mixture of glacial acetic acid and sulphuric acid under nitrogen. Alternatively, the isatin derivative was reacted at room temperature with a benzene derivative in triflic acid under nitrogen (see Klumpp et al. J. Org. Chem. (1998), 63, 4481-84). Thioamide derivatives of the final compounds (Q=S and n=1) were obtained by reacting the corresponding amides (Q=O and n=1) with Lawesson's reagent as described in Organic Synthesis Coll. Vol. VII, p 372.

Protocol D: Preparation of the Final Compounds

(a)

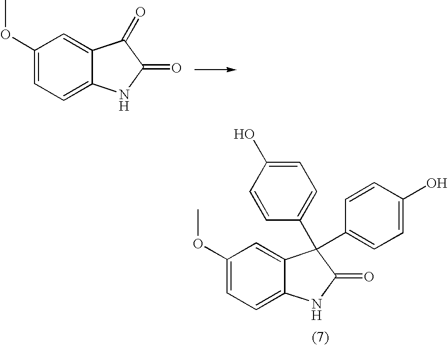

To a suspension of phenol (0.28 g, 2.9 mmol) and 5-methoxy-1H-indole-2,3-dione (0.24 g (1.3 mmol) in glacial acetic acid (1.5 ml) under nitrogen was added sulphuric acid (18.3 M, 0.145 mL). The mixture was heated at 100° C. for 2 hours. Crude reaction mixture was diluted with water and extracted with ethyl acetate (2×). The organic layer was dried (Na₂SO₄) and concentrated under reduced pressure to yield a brown solid. This solid was mixed with DCM:AcOEt (9:1) (3×) and gave 0.08 g (0.35 mmol, 18%) of 3,3-bis-(4-hydroxy-phenyl)-5-methoxy-1,3-dihydro-indol-2-one (7). LCMS m/z 348.19 [M+H]⁺ @ R_{T} 1.09 min, 100%. δ_{H} (400 MHz, Methanol-d4) 6.92 (4H, d, J=8.80 Hz), 6.79-6.82 (1H, m), 6.69-6.73 (1H, m), 6.61 (5H, m), 3.62 (3H, s).
(b)

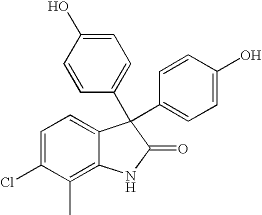

Phenol (15.3 g, 163.6 mmol) and 6-chloro-7-methyl-1H-indole-2,3-dione (16.0 g, 81.8 mmol) were suspended in glacial acetic acid (82 ml) and sulphuric acid (18.3 M, 8.8 mL) under nitrogen atmosphere. The reaction mixture was heated at 85° C., after 2 hour left cool to room temperature, diluted in ethyl acetate and washed with water (3×). The organic phase was dried over Na₂SO₄ and concentrated under reduced pressure. The crude material was purified by re-crystallization from toluene: ethyl acetate (20 volume: 1 volume) to yield 13.3 g of yellow solid containing sole toluene. Dried overnight in high vacuum at 45° C. to yield 10.65 g (29.2 mmol, 38%) of 6-chloro-3,3-bis-(4-hydroxy-phenyl)-7-methyl-1,3-dihydro-indol-2-one (3) as a white solid. LCMS (λ 215 nm, BDS-Hypersil C₁₈, 50 mm×2.1 mm, 5μ, 2.5 minute) m/z 366.3 [(Cl³⁵) M+H]⁺ @ R_{T} 1.3 min, 100%. δ_{H} (400 MHz, DMSO-d6) 10.9 (1H, s), 9.5 (2H, s), 7.1 (1H, d, J=9.8), 7.05 (1H, d, J=9.6), 6.95 (4H, d, J=10.2), 6.7 (4H, d, J=10.2), 2.35 (3H, s).
Protocol E: Preparation of the Final Compounds

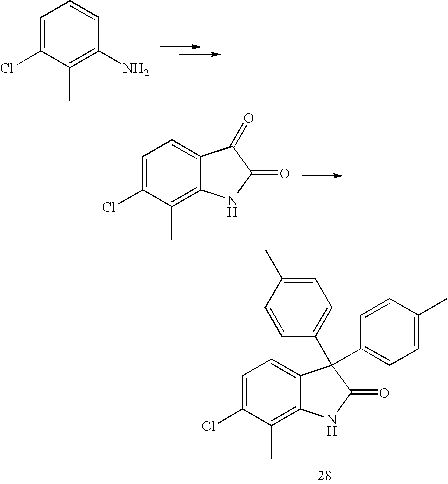

To a well stirred suspension of 6-chloro-7-methyl-1H-indole-2,3-dione (0.15 g, 0.76 mmol) in toluene (anhydrous) (1 mL) was added trifluoromethane sulfonic acid (1.25 mL). The tube was sealed and the mixture was stirred at room temperature for 12 hours. The dark brown reaction mixture was then slowly poured over ice (˜10 g) and left standing for 10 minutes. The formed precipitate was collected by filtration, washed with cold water (3×100 mL), dried under vacuum. Purification by flash column chromatography (gradient elution with EtOAc/Heptane (1:9 to 1:1)) followed by recrystallisation (MeOH/EtOAc) gave 25.2 mg (0.07 mmol, 9%) of 6-chloro-7-methyl-3,3-di-p-tolyl-1,3-dihydro-indol-2-one (28) as a light brown solid.

LCMS (BDS-Hypersil C₁₈, 50 mm×2.1 mm, 5μ, 2.5 minutes) m/z major 362.12 [MH]⁺ and minor 403.17 [MH+MeCN]⁺ @ retention time 2.18 minutes, 100% by UV at 215 nm.

δ_{H} (400 MHz, DMSO-d6) 2.24 (6H, s) 2.28 (3H, s) 7.00-7.03 (5H, m) 7.05-7.12 (5H, m) 10.96 (1H, s).

The following compounds were all prepared according to Protocols D or E, unless otherwise specified.

### 6-Chloro-3,3-bis-(4-hydroxy-phenyl)-7-methyl-1,3-dihydro-indol-2-one (3)

See protocol D.

### 6-Chloro-3,3-bis-(4-hydroxy-phenyl)-7-methyl-5-nitro-1,3-dihydro-indol-2-one (5)

LCMS m/z 411.1 [(Cl³⁵) M+H]⁺ @ R_{T} 1.26 min, 93%. δ_{H} (400 MHz, DMSO-d6) 7.48 (1H, s), 6.96-6.96 (4H, m), 6.66-6.59 (4H, m), 2.35 (3H, s).

### 5-Amino-6-chloro-3,3-bis-(4-hydroxy-phenyl)-7-methyl-1,3-dihydro-indol-2-one (6)

To a solution of 5 (0.1 g, 0.24 mmol) in methanol (2 mL) was added Pd/C (10% w/w, 0.03 g). The black mixture was stirred under hydrogen at room temperature for 16 hours. The catalyst was removed by filtration, and the solvent was removed under reduced pressure to yield 0.084 g (0.22 mmol, 92%) of 5-Amino-6-chloro-3,3-bis-(4-hydroxy-phenyl)-7-methyl-1,3-dihydro-indol-2-one (6). LCMS m/z 381.16 [(Cl³⁵) M+H]⁺ @ R_{T} 0.94 min, 84%. δ_{H} (400 MHz, DMSO-d6) 11.7 (1H, s), 8.1 (1H, s), 2.3 (3H, s).

### 3,3-Bis-(4-hydroxy-phenyl)-5-methoxy-1,3-dihydro-indol-2-one (7)

See protocol D.

### 3,3-Bis-(4-hydroxy-phenyl)-5-trifluoromethoxy-1,3-dihydro-indol-2-one (8)

LCMS m/z 402.12 [M+H]⁺ @ R_{T} 1.27 min, 96%. δ_{H} (400 MHz, DMSO-d6) 10.78 (1H, s), 9.43 (2H, s), 7.23 (1H, d, J=8.56), 7.17 (1H, s), 6.99 (1H, d, J=8.56), 6.93 (4H, d, J=8.80), 6.66 (4H, d, J=8.56).

### 3,3-Bis-(4-hydroxy-phenyl)-5,7-dimethyl-1,3-dihydro-indol-2-one (9)

LCMS m/z 346.19 [M+H]⁺ @ R_{T} 1.24 min, 92%. δ_{H} (400 MHz, DMSO-d6) 10.39 (1H, s), 9.25 (2H, s), 6.8 (4H, d, J=8.6), 6.70 (1H, s), 6.68 (1H, s), 6.52 (4H, d, J=8.6), 2.09 (6H, s).

### 3,3-Bis-(4-hydroxy-phenyl)-2-oxo-2,3-dihydro-1H-indole-7-carboxylic acid (10)

LCMS m/z 362.13 [M+H]⁺ @ R_{T} 1.06 min, 90%. δ_{H} (400 MHz, DMSO-d6) 10.11 (1H, s), 9.43 (2H, s), 7.71 (1H, dd, J=8.1, 1.2), 7.38 (1H, dd, J=7.3, 0.7), 7.08 (1H, t, J=7.8), 6.92 (4H, d, J=8.8), 6.67 (4H, d, J=8.8).

### 5-Chloro-3,3-bis-(4-hydroxy-phenyl)-1,3-dihydro-indol-2-one (11)

LCMS m/z 352.11 [(Cl³⁵) M+H]⁺ @ R_{T} 1.21 min, 100%. δ_{H} (400 MHz, DMSO-d6) 10.72 (1H, s), 9.42 (2H, s), 7.25 (1H, dd, J=8.2, 2.1), 7.18 (1H, d, J=2.2), 6.89-6.95 (5H, m), 6.68 (4H, d, J=8.6).

### 5-Fluoro-3,3-bis-(4-hydroxy-phenyl)-1,3-dihydro-indol-2-one (12)

LCMS m/z 336.16 [M+H]⁺ @ R_{T} 1.14 min, 90%. δ_{H} (400 MHz, DMSO-d6) 10.61 (1H, s), 9.41 (2H, s), 7.00-7.10 (2H, m), 6.93 (4H, d, J=8.6), 6.89 (1H, dd, J=8.4, 4.5), 6.67 (4H, d, J=8.8).

### 3,3-Bis-(4-hydroxy-phenyl)-5-nitro-1,3-dihydro-indol-2-one (13)

LCMS m/z 362.86 [M+H]⁺ @ R_{T} 1.25 min, 93%. δ_{H} (400 MHz, DMSO-d6) 11.31 (1H, s), 9.48 (2H, s), 8.19 (1H, dd, J=8.7, 2.3), 7.90 (1H, d, J=2.2), 7.12 (1H, d, 3=8.8), 6.94 (4H, d, J=8.8), 6.70 (4H, d, J=8.8).

### 5-Chloro-3,3-bis-(4-hydroxy-phenyl)-7-methyl-1,3-dihydro-indol-2-one (14)

LCMS m/z 365.92 [(Cl³⁵) M+H]⁺ @ R_{T} 1.36 min, 91%. δ_{H} (400 MHz, DMSO-d6) 10.77 (1H, s), 9.41 (2H, s), 7.10 (1H, d, 3=1.5), 6.98 (1H, d, J=1.9), 6.91 (4H, d, 3=8.6), 6.67 (4H, d, 3=8.6), 2.22 (3H, s).

### 3,3-Bis-(4-hydroxy-phenyl)-5-methyl-1,3-dihydro-indol-2-one (15)

LCMS m/z 331.97 [M+H]⁺ @ R_{T} 1.37 min, 91%. δ_{H} (400 MHz, DMSO-d6) 10.42 (1H, s), 9.33 (2H, s), 6.90-6.97 (2H, m), 6.88 (4H, d, 3=8.6), 6.75 (1H, d, 3=7.8), 6.62 (4H, d, J=8.8), 2.17 (3H, s).

### 5-Bromo-3,3-bis-(4-hydroxy-phenyl)-1,3-dihydro-indol-2-one (16)

LCMS m/z 396.05 [(Br⁷⁹) M+H]⁺ @ R_{T} 1.14 min, 94%. δ_{H} (400 MHz, MeOD) 7.28 (1H, dd, J=8.3, 2.0), 7.14 (1H, d, J=2.0), 6.88-6.92 (4H, m), 6.81 (1H, d, 3=8.3), 6.60-6.64 (4H, m).

### 3,3-Bis-(4-hydroxy-phenyl)-5-iodo-1,3-dihydro-indol-2-one (17)

LCMS m/z 444.01 [M+H]⁺ @ R_{T} 1.70 min, 100%. δ_{H} (250 MHz, MeOD) 6.72-6.85 (5H, m) 6.99-7.08 (5H, m) 7.15-7.21 (1H, m) 7.28 (1H, t, 3=7.23 Hz) 7.41-7.52 (2H, m) 7.60 (1H, dd, 3=8.23, 1.65 Hz).

### 5-Amino-3,3-bis-(4-hydroxy-phenyl)-1,3-dihydro-indol-2-one (18)

LCMS m/z 333.13 [M+H]⁺ @ R_{T} 1.29 min, 90%. δ_{H} (250 MHz, Methanol-D4) 6.71 (4H, d, 3=8.60 Hz) 6.98-7.05 (4H, m) 7.12 (1H, d, J=8.23 Hz) 7.20 (1H, d, 3=1.83 Hz) 7.26-7.33 (1H, m).

### 5-Amino-3,3-bis-(4-hydroxy-phenyl)-7-methyl-1,3-dihydro-indol-2-one (19)

LCMS m/z 347.14 [M+H]⁺ @ R_{T} 1.28 min, 100%. δ_{H} (400 MHz, Methanol-D4) 7.02 (4H, d, 3=8.8 Hz), 6.68 (4H, d, 3=8.8 Hz), 6.42-6.52 (2H, m), 2.21 (3H, s).

### 6-Bromo-3,3-bis-(4-hydroxy-phenyl)-7-methyl-1,3-dihydro-indol-2-one (20)

LCMS m/z 410.04 [M+H]⁺ @ R_{T} 1.39 min, 94%. δ_{H} (400 MHz, Methanol-D4) 7.22 (1H, d, 3=7.8 Hz), 7.00 (4H, d, 3=8.8 Hz), 6.85 (1H, d, 3=7.8 Hz), 6.69 (4H, d, J=8.8 Hz), 2.35 (3H, s).

### 3,3-Bis-(4-hydroxy-phenyl)-7-fluoro-1,3-dihydro-indol-2-one (21)

LCMS m/z 336.11 [M+H]⁺ @ R_{T} 1.15 min, 97%. δ_{H} (400 MHz, Methanol-D4) 6.85-6.97 (7H, m), 6.60 (4H, d, 3=8.8 Hz).

### 3,3-Bis-(4-hydroxy-phenyl)-7-methoxy-1,3-dihydro-indol-2-one (22)

LCMS m/z 348.13 [M+H]⁺ @ R_{T} 1.14 min, 94%. δ_{H} (400 MHz, Methanol-D4) 6.95-7.06 (5H, m), 6.89 (1H, d, J=8.3 Hz), 6.75 (1H, d, J=7.8 Hz), 6.68 (4H, d, J=8.8 Hz), 3.89 (3H, s).

### 4,7-Dichloro-3,3-bis-(4-hydroxy-phenyl)-1,3-dihydro-indol-2-one (23)

LCMS m/z 386.04 [M+H]⁺ @ R_{T} 1.35 min, 97%. δ_{H} (400 MHz, Methanol-D4) 7.29 (1H, d, J=8.8 Hz), 7.06 (4H, d, J=8.8 Hz), 6.97 (1H, d, J=8.8 Hz), 6.71 (4H, d, 7=8.8 Hz).

### 6-Chloro-3,3-bis-(4-hydroxy-phenyl)-1,7-dimethyl-1,3-dihydro-indol-2-one (24)

LCMS m/z 380.11 [M+H]⁺ @ R_{T} 1.49 min, 100%. δ_{H} (400 MHz, Methanol-D4) 7.12 (1H, d, 7=7.8 Hz), 6.85-7.02 (5H, m), 6.60-6.72 (4H, m), 3.57 (3H, s), 2.69 (3H, s).

### 6-Chloro-3,3-bis-(4-fluoro-phenyl)-7-methyl-1,3-dihydro-indol-2-one (25)

δ_{H} (400 MHz, Methanol-D4) 7.15-7.30 (4H, m), 6.97-7.13 (6H, m), 2.34 (3H, s).

### 3,3-Bis-(4-hydroxy-phenyl)-7-(morpholine-4-carbonyl)-1,3-dihydro-indol-2-one (26)

To 10 (1 eq) dissolved in dimethylformamide was added SOCl₂ (3 eq) at 0° C. The mixture was stirred for 1 hour and evaporated to remove excess SOCl₂. Morfoline (3 eq) was added and the reaction mixture was left for 3 hours at room temperature. The solvent was removed in vacuo and the 26 purified by filtration through a pad of silica using dichloromethane-MeOH as eluent. LCMS m/z 431.04 [M+H]⁺ @ R_{T} 1.13 min, 90%. δ_{H} (400 MHz, Methanol-D4) 7.19-7.29 (2H, m), 7.11 (1H, m), 6.97-7.05 (4H, m), 6.64-6.75 (4H, m), 3.69 (8H, brs).

### 3,3-Bis-(4-hydroxy-phenyl)-1,3-dihydro-pyrrolo[3,2-c]pyridin-2-one (27)

LCMS (BDS-Hypersil C₁₈, 50 mm×2.1 mm, 5μ, 2.5 minutes) m/z 319.28 [MH]⁺ @ R_{T} 0.76 min, 100% by UV at 215 nm. δ_{H} (400 MHz, CD₃OD) 6.63 (4H, d, J=8.6 Hz), 6.93 (4H, d, J=8.8 Hz), 6.95 (1H, d, 75.4 Hz), 8.10 (1H, s), 8.24 (1H, d, J=5.4 Hz).

### 6-Chloro-7-methyl-3,3-di-p-tolyl-1,3-dihydro-indol-2-one (28)

See protocol E.

### 3,3-Bis-(4-hydroxy-phenyl)-3,6,7,8-tetrahydro-1H-1-aza-as-indacen-2-one (29)

Phenol (1.0 g, 10.84 mmol) was added to crude 3,3-dibromo-1,3-dihydro-pyrrolo[2,3-b]pyridine-2-one (0.15 g, 0.51 mmol, prepared according to Parrick et al. Tet. Lett. (1984), 25, 3099) and the mixture was heated to 100° C. for 10 minutes, allowed to cool to room temperature and the excess phenol removed by flash chromatography. The silica adsorbed product was isolated by washing with methanol and concentrating under reduced pressure. The pH was adjusted to approximately 6 using sodium carbonate solution and the crude product isolated by evaporation under reduced pressure. Purification by preparative HPLC provided the title compound (29) (3 mg, 2%). LCMS m/z 358.35 [M+H]⁺ @ R_{T} 1.26 min, 89%. δ_{H} (400 MHz, DMSO-D6) 10.62 (1H, s), 9.35 (2H, s), 6.90-6.95 (4H, m), 6.82-6.90 (2H, m), 6.62-6.68 (4H, m), 2.75-2.87 (4H, m), 1.98-2.08 (2H, m).

### 7-Bromo-3,3-bis-(4-hydroxy-phenyl)-1,3-dihydro-indol-2-one (30)

LCMS m/z 398.22 [M+H]⁺ @ R_{T} 1.22 min, 100%.

### N-{4-[6-Chloro-3-(4-methanesulfonylamino-phenyl)-7-methyl-2-oxo-2,3-dihydro-1H-indol-3-yl]-phenyl}-methanesulfonamide (31)

LCMS m/z 520.27 [M+H]⁺ @ R_{T} 1.30 min, 96%. δ_{H} (400 MHz, DMSO-D6) 11.00 (1H, s), 9.78 (2H, s), 6.85-7.37 (10H, m), 2.97 (6H, s), 2.28 (3H, s).

### 7-Ethyl-3,3-bis-(4-hydroxy-phenyl)-1,3-dihydro-indol-2-one (32)

LCMS m/z 345.97 [M+H]⁺ @ R_{T} 1.30 min, 100%.

### 3,3-Bis-(4-hydroxy-phenyl)-7-iodo-1,3-dihydro-indol-2-one (33)

LCMS m/z 443.82 [M+H]⁺ @ R_{T} 1.37 min, 100%.

### 3,3-Bis-(4-hydroxy-phenyl)-7-chloro-1,3-dihydro-indol-2-one (34)

LCMS m/z 351.56 [M+H]⁺ @ R_{T} 1.33 min, 100%. δ_{H} (400 MHz, Methanol-D4) 7.23 (1H, dd, J=8.3, 1.0 Hz), 7.05-7.11 (1H, m), 6.96-7.04 (5H, m), 6.70 (4H, d, J=8.8 Hz).

### 3,3-Bis-(4-hydroxy-phenyl)-7-trifluoromethyl-1,3-dihydro-indol-2-one (35)

LCMS m/z 387.98 [M+H]⁺ @ R_{T} 1.35 min, 94%. δ_{H} (400 MHz, Methanol-D4) 7.49 (1H, d, J=8.3 Hz), 7.38 (1H, d, J=7.3 Hz), 7.17 (1H, t, J=7.6 Hz), 7.00 (4H, d, J=8.8 Hz), 6.71 (4H, d, J=8.8 Hz).

### Acetic acid 4-[3-(4-acetoxy-phenyl)-6-chloro-7-methyl-2-oxo-2,3-dihydro-1H-indol-3-yl]-phenyl ester (36)

LCMS m/z 450.10 [M+H]⁺ @ R_{T} 1.63 min, 94%.

### 3,3-Bis-(4-hydroxy-phenyl)-6-methoxy-1,3-dihydro-indol-2-one (37)

LCMS m/z 348.22 [M+H]⁺ @ R_{T} 1.14 min, 98%. δ_{H} (400 MHz, Methanol-D4) 6.95-7.05 (5H, m), 6.63-6.74 (4H, m), 6.53-6.61 (2H, m), 3.77 (3H, s).

### 5,7-Difluoro-3,3-bis-(4-hydroxy-phenyl)-1,3-dihydro-indol-2-one (38)

LCMS m/z 353.95 [M+H]⁺ @ R_{T} 1.25 min, 100%. δ_{H} (400 MHz, Methanol-D4) 7.00 (4H, d, J=8.8 Hz), 6.93 (1H, td, 3=9.8, 2.0 Hz), 6.81 (1H, dd, J=8.1, 2.2 Hz), 6.72 (4H, d, J=8.8 Hz).

### 6-Fluoro-3,3-bis-(4-hydroxy-phenyl)-7-methyl-1,3-dihydro-indol-2-one (39)

LCMS m/z 349.98 [M+H]⁺ @ R_{T} 1.28 min, 100%. δ_{H} (400 MHz, Methanol-D4) 7.00 (4H, d, J=8.8 Hz), 6.93 (1H, dd, 3=8.3, 5.4 Hz), 6.61-6.76 (5H, m), 2.21 (3H, d, J=1.0 Hz).

### 3,3-Bis-(4-hydroxy-phenyl)-6-methoxy-7-methyl-1,3-dihydro-indol-2-one (40)

LCMS m/z 362.00 [M+H]⁺ @ R_{T} 1.35 min, 100%. δ_{H} (400 MHz, Methanol-D4) 7.00 (4H, d, J=8.8 Hz), 6.89 (1H, d, 3=8.3 Hz), 6.67 (4H, d, J=8.8 Hz), 6.59 (1H, d, 3=8.3 Hz), 3.80 (3H, s), 2.14 (3H, s).

### 6,7-Difluoro-3,3-bis-(4-hydroxy-phenyl)-1,3-dihydro-indol-2-one (41)

LCMS m/z 353.96 [M+H]⁺ @ R_{T} 1.35 min, 96%. δ_{H} (400 MHz, Methanol-D4) 7.00 (4H, d, 3=8.8 Hz), 6.82-6.96 (2H, m), 6.70 (4H, d, 3=8.8 Hz).

### 6-Chloro-7-fluoro-3,3-bis-(4-hydroxy-phenyl)-1,3-dihydro-indol-2-one (42)

LCMS m/z 369.95 [M+H]⁺ @ R_{T} 1.30 min, 100%. δ_{H} (400 MHz, Methanol-D4) 7.10 (1H, dd, 3=8.1, 6.6 Hz), 7.00 (4H, d, 3=8.8 Hz), 6.95 (1H, d, J=8.8 Hz), 6.70 (4H, d, 3=8.8 Hz).

### 3,3-Bis-(4-hydroxy-phenyl)-2-oxo-2,3-dihydro-1H-indole-7-carbonitrile (43)

Compound 33 (0.35 g, 0.79 mmol) was treated with zinc cyanide (0.14 g, 1.18 mmol) and tetrakis(triphenylphosphine)palladium(0) (0.09 g, 10%) in anhydrous DMF (5 mL). The reaction mixture was degassed by nitrogen bubbling for 15 minutes. The reaction was then heated to 100° C. overnight under nitrogen. After cooling to room temperature the reaction was quenched with saturated aqueous NaHCO₃. The resulting cloudy suspension was filtered and the filtrate dissolved in a mixture of toluene and ethylacetate (1:1), washed with aq. NaHCO₃ (saturated) (2×), water (2×) and dried over sodium sulphate. After filtration the organic layer was concentrated under reduced pressure to give the crude product. Re-treatment of the crude material was carried out a further two times with the same amounts and conditions as above. The compound was initially purified by flash column chromatography (DCM: MeOH with gradient elution 95:5 to 9:1) followed by preparative HPLC to afford the desired compound (43) as a white solid (0.014 g, 5%). LCMS m/z 343.07 [M+H]⁺ @ R_{T} 1.15 min, 97%. δ_{H} (400 MHz, Methanol-D4) 7.51 (1H, dd, J=7.8, 1.0 Hz), 7.41 (1H, dd, J=7.8, 1.0 Hz), 7.13 (1H, t, J=7.8 Hz), 6.99 (4H, d, J=8.8 Hz), 6.71 (4H, d, J=8.8 Hz).

### 5-Fluoro-3,3-bis-(4-hydroxy-phenyl)-7-methyl-1,3-dihydro-indol-2-one (44)

LCMS m/z 350.29 [M+H]⁺ @ R_{T} 1.20 min, 95%. δ_{H} (400 MHz, Methanol-D4) 7.00 (4H, d, J=8.8 Hz), 6.82 (1H, dd, J=10.5, 2.2 Hz), 6.62-6.75 (5H, m), 2.30 (3H, s).

### 3,3-Bis-(4-hydroxy-phenyl)-5-methoxy-7-methyl-1,3-dihydro-indol-2-one (45)

LCMS m/z 362.25 [M+H]⁺ @ R_{T} 1.16 min, 91%. δ_{H} (400 MHz, Methanol-D4) 7.01 (4H, d, J=8.8 Hz), 6.69 (4H, d, J=8.8 Hz), 6.64 (1H, d, J=2.5 Hz), 6.53 (1H, d, J=2.5 Hz), 3.68 (3H, s), 2.28 (3H, s).

### 3,3-Bis-(4-hydroxy-phenyl)-1,3-dihydro-pyrrolo[2,3-b]pyridin-2-one (46)

LCMS m/z 319.27 [M+H]⁺ @ R_{T} 0.97 min, 100%. δ_{H} (400 MHz, Methanol-D4) 8.10 (1H, dd, J=4.9, 1.5 Hz), 7.55 (1H, dd, J=7.3, 1.5 Hz), 6.93-7.11 (5H, m), 6.71 (4H, d, J=8.8 Hz).

### 6-Fluoro-3,3-bis-(4-hydroxy-phenyl)-1,3-dihydro-indol-2-one (47)

LCMS m/z 336.27 [M+H]⁺ @ R_{T} 1.17 min, 100%. δ_{H} (400 MHz, Methanol-D4) 7.04-7.18 (1H, m), 7.00 (4H, d, J=8.80 Hz), 6.62-6.79 (6H, m).

### N-[3,3-Bis-(4-hydroxy-phenyl)-2-oxo-2,3-dihydro-indol-1-yl]-acetamide (48)

LCMS m/z 375.27 [M+H]⁺ @ R_{T} 1.08 min, 100%. δ_{H} (400 MHz, Methanol-D4) 7.25-7.33 (1H, m), 7.14-7.19 (1H, m), 7.12 (1H, dd, J=7.3, 1.0 Hz), 7.08 (4H, d, J=8.8 Hz), 6.95 (1H, d, J=7.8 Hz), 6.69 (4H, d, J=8.8 Hz), 2.17 (3H, s).

### 5-[3,3-Bis-(4-hydroxy-phenyl)-7-methyl-2-oxo-2,3-dihydro-1H-indol-6-yloxy]-pentanoic acid methyl ester (49)

LCMS m/z 462.28 [M+H]⁺ @ R_{T} 1.41 min, 97%.

### 5-[3,3-Bis-(4-hydroxy-phenyl)-7-methyl-2-oxo-2,3-dihydro-1H-indol-6-yloxy]-pentanoic acid (50)

LCMS m/z 448.32 [M+H]⁺ @ R_{T} 1.13 min, 95%. δ_{H} (400 MHz, Methanol-D4) 7.01 (4H, d, J=9.0 Hz), 6.86 (1H, d, J=8.2 Hz), 6.67 (4H, d, J=8.8 Hz), 6.56 (1H, d, 3=8.4 Hz), 3.97 (2H, t, J=5.1 Hz), 2.36 (2H, t, J=6.4 Hz), 2.15 (3H, s), 1.72-1.91 (4H, m).

### 3,3-Bis-(4-hydroxy-phenyl)-6-methyl-1,3-dihydro-indol-2-one (51)

LCMS m/z 332.27 [M+H]⁺ @ R_{T} 1.90 min, 100%. δ_{H} (400 MHz, Methanol-D4) 6.92-7.08 (5H, m), 6.85 (1H, d, J=8.3 Hz), 6.80 (1H, s), 6.68 (4H, d, J=8.8 Hz), 2.33 (3H, s).

### 7-Chloro-3,3-bis-(4-hydroxy-phenyl)-6-methyl-1,3-dihydro-indol-2-one (52)

LCMS m/z 366.22 [M+H]⁺ @ 1.93 R_{T} min, 100%. δ_{H} (400 MHz, Methanol-D4) 7.00 (4H, d, J=8.8 Hz), 6.96 (2H, s), 6.69 (4H, d, J=8.8 Hz), 2.36 (3H, s).

### 5-Hydroxy-3,3-bis-(4-hydroxy-phenyl)-7-methyl-1,3-dihydro-indol-2-one (53)

LCMS m/z 348.26 [M+H]⁺ @ R_{T} 1.55 min, 100%.

### 3,3-Bis-(4-hydroxy-phenyl)-6,7-dimethyl-1,3-dihydro-indol-2-one (54)

LCMS m/z 346.30 [M+H]⁺ @ 1.85 R_{T} min, 100%. δ_{H} (400 MHz, Methanol-D4) 7.00 (4H, d, J=9.0 Hz), 6.84 (2H, s), 6.67 (4H, d, J=9.0 Hz), 2.27 (3H, s), 2.22 (3H, s).

Protocol F: Preparation of Final Products

General route for mono and mixed Friedel and Craft products via Grignard addition.

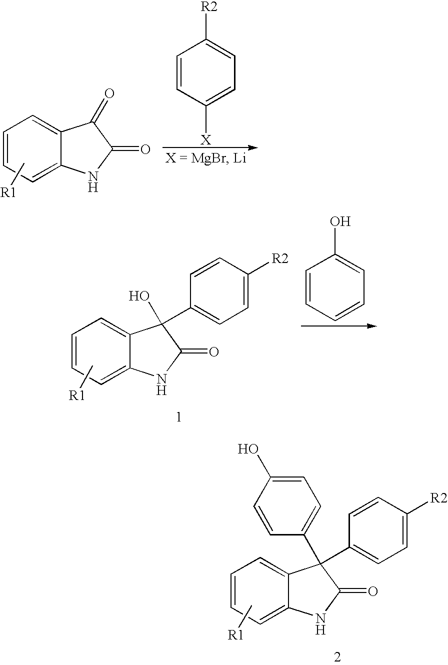

(a) Grignard Addition: To a stirred solution of isatin in dry tetrahydrofuran under nitrogen at −78° C. was added 3 eq. of Grignard reagent or 3 eq. of a freshly prepared solution of organo-lithium reagent. After 30 min, the dry-ice bath was removed on the reaction was left to reach room temperature over 4 to 14 hours. To the reaction mixture was then added water, to quench excess Grignard reagent, acidified to pH 1-2 with 1N HCl, extracted with EtOAc (2×), dried over Na₂SO₄, filtered and concentrated to yield the crude products as yellowish viscous oils which were either purified over silica (eluted with a gradient of Heptane/EtOAc from 95-5 to 1-1) to yield the desired racemic mixture of compound of the type 1 as solids or taken to the next step without purification.

(b) Friedel and Craft reaction: To a crude solution of tertiary alcohol in dichloroethane was added phenol (5 eq.) and p-TSA (7.5 eq.). The reaction mixture was heated to 90° C. for 3 hours and the reaction was cooled to room temperature. The solid (mainly insoluble p-TSA) was filtered off and washed (2×) with cold dichloroethane. The solution was concentrated and the remaining solid was purified over silica (eluted with a gradient of Heptane/EtOAc from 95-5 to 1-1) to yield the desired racemic mixture of product of the type 2 as solid.

The following compounds were all prepared according to Protocol F, unless otherwise specified.

### 6-Chloro-3-(4-hydroxy-phenyl)-7-methyl-3-p-tolyl-1,3-dihydro-indol-2-one (59)

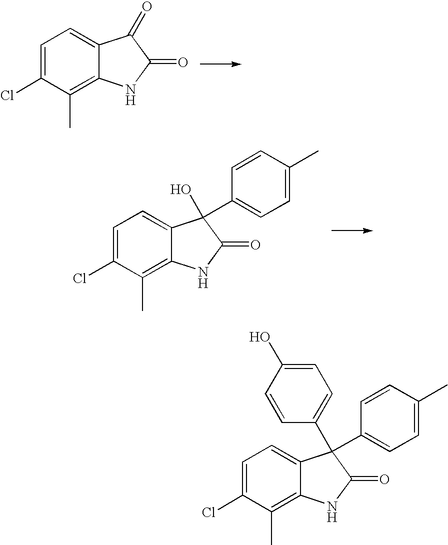

Intermediate: LCMS (λ 215 nm, BDS-Hypersil C₁₈, 50 mm×2.1 mm, 5μ, 2.8 minute method ref: MET/CR/0720) m/z 270 [M+H—H₂O]⁺ @ retention time 1.99 minute, 97%.

Final product (59): LCMS (λ 215 nm, BDS-Hypersil C₁₈, 50 mm×2.1 mm, 5μ, 2.5 minute method ref: MET/CR/0720) m/z 364 [M+H]⁺ @ retention time 1.64 minute, 100%. Overall yield 87% over 2 steps. δ_{H} (400 MHz, Methanol-d4) 2.28 (3H, s), 2.33 (3H, s), 6.69 (2H, d, J=8.8 Hz), 6.86-7.16 (8H, m).

### 6-Chloro-3-(4-hydroxy-phenyl)-3-(4-methoxy-phenyl)-7-methyl-1,3-dihydro-indol-2-one (60)

Intermediate: LCMS (λ 215 nm, BDS-Hypersil C₁₈, 50 mm×2.1 mm, 5μ, 2.8 minute method ref: MET/CR/0720) m/z 286[M+H—H₂O]⁺@ retention time 1.92 minute, 100%.

Final product (60): LCMS (λ 215 nm, BDS-Hypersil C₁₈, 50 mm×2.1 mm, 5μ, 2.5 minute method ref: MET/CR/0720) m/z 380 [M+H]⁺@ retention time 1.57 minute, 100%. Overall yield 60% over 2 steps. δ_{H} (400 MHz, Methanol-d4) 2.34 (3H, s), 3.75 (3H, s), 6.69 (2H, d, J=8.8 Hz), 6.83 (2H, d, J=9.3 Hz), 6.91-7.02 (3H, m), 7.04-7.14 (3H, m).

### 6,7-Difluoro-3-(4-hydroxy-phenyl)-3-p-tolyl-1,3-dihydro-indol-2-one (57)

Intermediate: LCMS (λ 215 nm, BDS-Hypersil C₁₈, 50 mm×2.1 mm, 5μ, 2.8 minute method ref: MET/CR/0720) m/z 258[M+H—H₂O]⁺ @ retention time 1.96 minute, 96%.

Final product: LCMS (λ 215 nm, BDS-Hypersil C₁₈, 50 mm×2.1 mm, 5μ, 2.8 minute method ref: MET/CR/0720) m/z 352 [M+H]⁺ @ retention time 2.06 minute, 98%. Overall yield 57% over 2 steps. δ_{H} (250 MHz, CDCl₃) 2.31 (3H, s), 4.76 (1H, s), 6.60-6.97 (4H, m), 7.03-7.15 (6H, m), 7.55 (1H, s).

### 6,7-Difluoro-3-(4-hydroxy-phenyl)-3-(4-methoxy-(phenyl)-,3-dihydro-indol-2-one (58)

Intermediate: LCMS (λ 215 nm, BDS-Hypersil C₁₈, 50 mm×2.1 mm, 5μ, 2.8 minute method ref: MET/CR/0720) m/z 274[M+H—H₂O]⁺ @ retention time 1.81 minute, 97%.

Final product (58): LCMS (λ 215 nm, BDS-Hypersil C₁₈, 50 mm×2.1 mm, 5μ, 2.8 minute method ref: MET/CR/0720) m/z 368[M+H]⁺ @ retention time 1.99 minute, 94%. Overall yield 14% over steps.

### 3-(4-Benzyloxy-phenyl)-6-chloro-3-(4-hydroxy-phenyl)-7-methyl-1,3-dihydro-indol-2-one

Intermediate: LCMS (λ 215 nm, BDS-Hypersil C₁₈, 50 mm×2.1 mm, 5μ, 2.8 minute method ref: MET/CR/0720) m/z 362[M+H—H₂O]⁺ @ retention time 2.16 minute, 88%. δ_{H} (400 MHz, Methanol-d4) 2.32 (3H, s), 5.05 (2H, s), 6.93 (2H, d, J=8.8 Hz), 6.96-7.02 (1H, m), 7.03-7.13 (1H, m), 7.20-7.46 (7H, m).

Final product: LCMS (λ 215 nm, BDS-Hypersil C₁₈, 50 mm×2.1 mm, 5μ, 2.5 minute method ref: MET/CR/0720) m/z 456-[MH]⁺ @ retention time 1.59 minute, 100%. Overall yield 11% over 2 steps. δ_{H} (400 MHz, Methanol-d4) 2.31 (3H, s), 3.83 (2H, d, J=2.5 Hz), 6.60-6.72 (3H, m), 6.77-6.89 (3H, m), 6.91-7.21 (9H, m).

### 3-(4-Benzyloxy-phenyl)-6,7-difluoro-3-(4-hydroxy-phenyl)-1,3-dihydro-indol-2-one

Intermediate: LCMS (λ 215 nm, BDS-Hypersil C₁₈, 50 mm×2.1 mm, 5μ, 2.8 minute method ref: MET/CR/0720) m/z 350[M+H—H₂O]⁺ @ retention time 2.07 minute, 94%. δ_{H} (400 MHz, Methanol-d4), 5.06 (2H, s), 6.82-7.01 (4H, m), 7.24-7.31 (3H, m), 7.34 (2H, t, J=7.3 Hz), 7.38-7.45 (2H, m).

Final product: LCMS (λ 215 nm, BDS-Hypersil C₁₈, 50 mm×2.1 mm, 5μ, 2.8 minute method ref: MET/CR/0720) m/z 444[M+H—H₂O]⁺ @ retention time 2.03 minute, 88%. Overall yield 24% over 2 steps.

Protocol G—Debenzylation/Dehalogenation

### 3-Hydroxy-3-(4-hydroxy-phenyl)-7-methyl-1,3-dihydro-indol-2-one

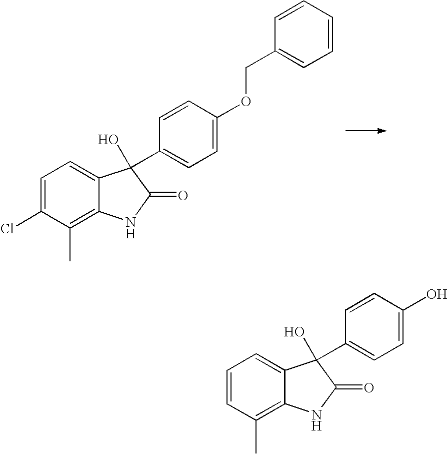

A clean sample of tertiary alcohol (40.9 mg), Pd/C (10 wt %) in methanol was submitted to hydrogenation conditions. The reaction was monitored by LCMS. After 14 h at room temperature, the palladium on charcoal was filtered off and washed with methanol. The combined organic layer were concentrated, and the crude product was purified by silica (with a gradient of Heptane/EtOAc from 85-15 to 1-1) to yield the racemic target compound (4.5 mg, 16% yield) as a solid. LCMS (λ 215 nm, BDS-Hypersil C₁₈, 50 mm×2.1 mm, 5μ, 2.5 minute method ref: MET/CR/0720) m/z 238[M+H—H₂O]⁺ @ retention time 1.26 minute, 100%

### 6,7-Difluoro-3-hydroxy-3-(4-hydroxy-phenyl)-1,3-dihydro-indol-2-one

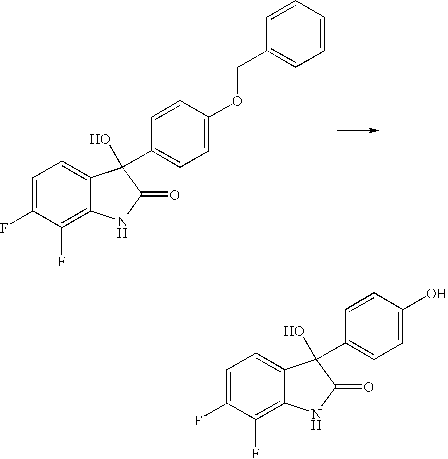

A clean sample of tertiary alcohol (41.8 mg), Pd/C (10 wt %) in methanol was submitted to hydrogenation conditions. The reaction was monitored by LCMS. After 14 h at room temperature, the palladium on charcoal was filtered off and washed with methanol. The combined organic layer were concentrated, and the crude product was purified by silica (with a gradient of Heptane/EtOAc from 85-15 to 1-1) to yield the racemic target compound (5.5 mg, 17% yield) as a solid. LCMS (λ 215 nm, BDS-Hypersil C₁₈, 50 mm×2.1 mm, 5μ, 2.5 minute method ref: MET/CR/0720) m/z 260[M+H—H₂O]⁺ @ retention time 1.29 minute, 100%.

### Example 2

### Cell Proliferation

Inhibition of the proliferation of human cancer cells is widely used to predict the anti-cancer potential of novel chemicals. Typically, human cancer cell lines derived from tumour material are maintained in monolayer cultures and test chemicals are added for varying durations. Test compounds with anti-cancer potential are expected to reduce proliferation and thereby reduce cell number relative to vehicle treated control cell cultures. Cell number can be monitored by cell counting, determining metabolic rate (e.g. metabolic reduction of tetrazolium salts such as (3-(4,5-dimethylethiazol-2-yl)-2,5-diphenyltetrazolium bromide or Alamar Blue), quantifying DNA content (using DNA binding dyes such as BODIPY-FL-14-dUTP) or measuring nucleotide incorporation into DNA (e.g. radiolabelled thymidine or bromo-deoxyuridine incorporation).

One important consideration is whether any inhibitory effects of test compounds are specific to cancer cell proliferation or are due to general inhibition of cell proliferation. This issue can be addressed using paired cell lines; for example, the effects of test compounds on the proliferation of transformed cancer cell lines can be compared with the effects of test compounds on the proliferation of untransformed cells from the same tissue source. Alternatively, phenotypic differences between cancer cell lines can be exploited to evaluate the selectivity of test compounds. For example, the anti-proliferative effects of some compounds are only apparent in certain sub-types of human breast cancer cell lines (e.g. breast cancer cell lines with PTEN gene mutations or gene amplification of the p70S6K protein kinase), but not in breast cancer cell lines that do not exhibit this phenotype (Noh et al. (2004) Clinical Cancer Research 10, 1013-1023; Yu et al. (2001) Endocrine-Related Cancer 8, 249-258). The selectivity of test compounds in the latter models is associated with the mechanism of compound action and is related to the presence, absence or relative abundance of the protein target of the test compound in the relevant cell lines.

Method

Compound effects were evaluated on the proliferation of MDA-468 and MDA-231 human breast cancer cells. Cells were maintained in growth medium: RPMI 1640 containing 100% foetal bovine serum and 1% pen/strep. Cells were split 1:4 or 1:8 twice a week when 90% confluent. For the cell proliferation assay, cells were plated at 8000 cell/well into 96 well black Packard Viewplates in growth medium. After 1 day, the growth medium was replaced with growth medium containing test compounds or vehicle, and cells were maintained in culture for a further 2 days. Growth medium was then removed and replaced with 150 μl of alamarBlue in RPMI medium containing 1% pen/strep. Following 120 minutes incubation at 37° C., fluorescent intensity was read using a plate reader.

Results

The concentration (in micromolar) of compounds of general formula (I) required to inhibit the proliferation of MDA-468 and MDA-231 human breast cancer cells by 50% (IC₅₀) are shown in FIG. 1. The results shown in FIG. 1 demonstrate the ability of the compounds of the general formula (I) to inhibit the proliferation of MDA-468 human breast cancer cells at lower concentrations as those required to inhibit proliferation of MDA-231 human breast cancer cells.

### Example 3

### Protein Synthesis Studies

The purpose of these studies as to investigate compounds of the general formula (I) have effect on protein synthesis, measured as ¹⁴C-Leucine uptake or incorporation into proteins. As described in “Leucine Uptake [¹⁴C] Cytostar-T assay, Amersham Biosciences” (CFA773).

MDA-MB-231 and MDA-MB-468 cells were seeded at 8000 cells/well in CytoStar-T 96-well microplates. And incubated overnight in growth medium. The next day medium was carefully aspirated (8-channel Vacuboy) and 50 μL of fresh pre-warmed medium (10% FCS, 10 mM HEPES pH 7.2-7.5) was added. Cells were allowed to equilibrate at 37° C. for 60 min. Test compounds were added in 50 μL medium and ¹⁴C-leucine was added in 100 μL medium (0.5 μCi mL−1 final). Plates were sealed with transparent, adhesive foil. Plates were then incubated in a 37° C. for 6 h in a humidified incubator. Incorporation of radioactive leucine into proteins (a measure of protein synthesis) was then read by coincidence scintillation (counts per minute (CPM)) using a Wallac Microbeta detector at the indicated time-intervals. A reading a t=0 (5 min after sealing plates) for each well is subtracted as background.

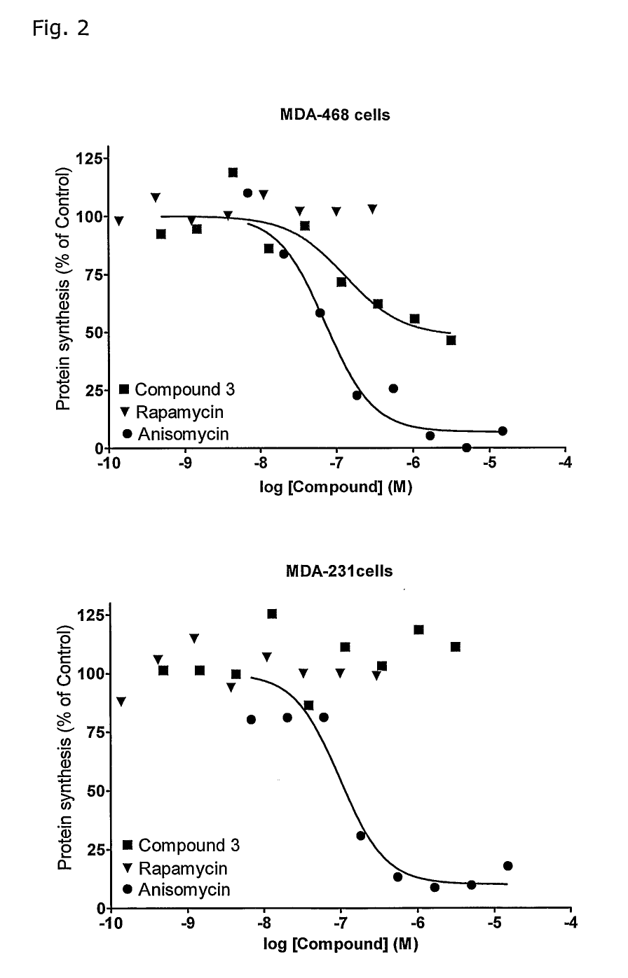

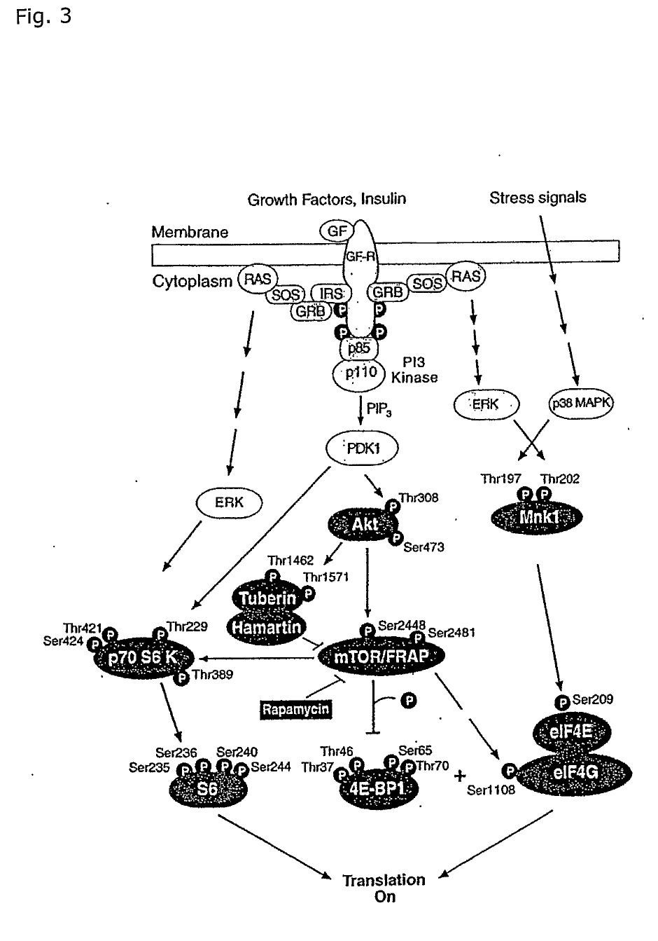

The results are shown in FIG. 2 measured after 6 hours.

The results indicate that Compound 3 significantly inhibits ¹⁴C-Leucine incorporation in MDA-MB-468 in a concentration dependent manner observed after 240 min compound incubation and up to 22 hours. IC₅₀ is estimated to 100 nM (240 min to 22 hours). Interestingly, the effect seems to reach a plateau at the high concentrations corresponding to approx. ⅙ of total incorporated. This indicates that there is some proportion of the protein synthesis that Compound 3 is not able to affect.

No significant effect of Compound 3 was observed in MDA-MB-231 up to 430 min. At 22 hours a minor effect is observed at 30 μM. IC₅₀>>30 μM (22 hours).

The inhibitory effect of Compound 3 is therefore very specific for MDA-MB-468.

The control compounds Anisomycin and Cycloheximide (not shown) completely inhibit ¹⁴C-Leucine incorporation in both cell lines at all time-points (as opposed to Compound 3, see above).

### Example 4

### Western Blot Studies

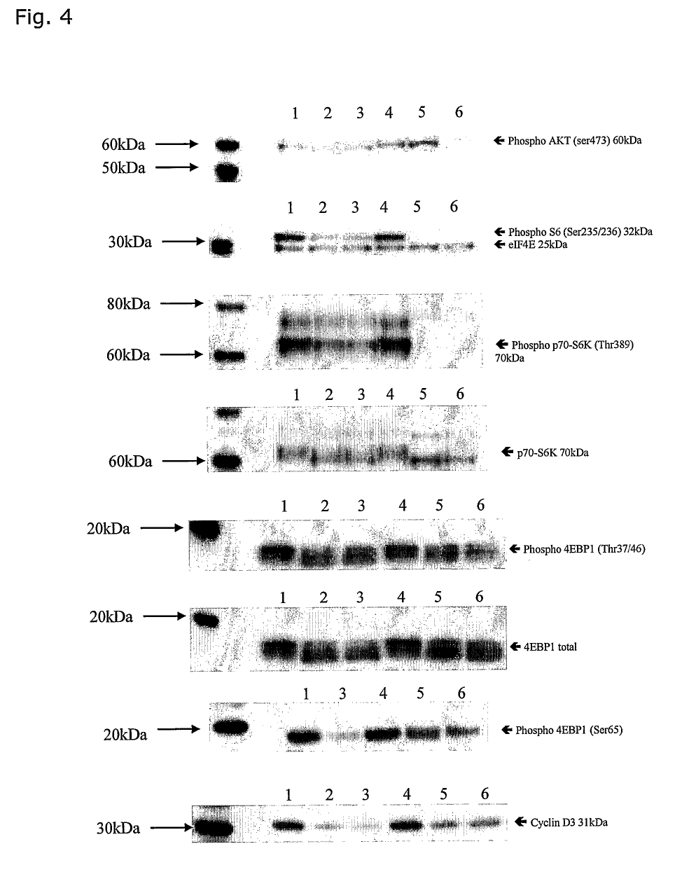

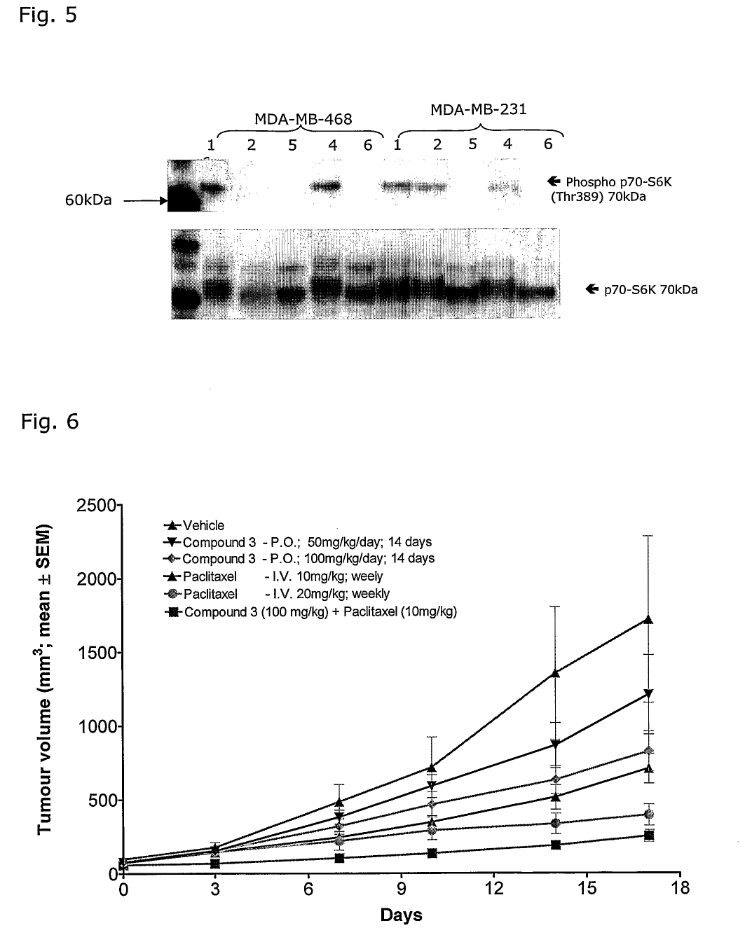

To investigate the mechanism of action of compounds of general formula (I) Western Blot studies were performed to investigate the activation state of pathways linked to the regulation of protein synthesis (see FIGS. 4 and 5).

Method

MDA-MB-468 cells (also called MDA-468) or MDA-MB-231 (also called MDA-231) were kept in culture and plated at 400,000 cells/well in 6 well cell culture plate. 16-24 hours after, the growth medium were shifted to growth medium containing compounds.

After 24 or 48 hours incubation with compounds, cells were washed with ice cold PBS buffer and harvested in lysis buffer: Cytobuster reagent (Novagen) containing phosphatase inhibitor cocktail 1 and 2 and protease inhibitor cocktail (Sigma). Samples containing an equal amount of protein were loaded onto 7% Tris Acetate gels, 10% Bis-Tris in MES buffer or 12% Bis-Tris gels using MOPS running buffer (Invitrogen). Following electrophoresis the samples were blotted onto a PVDF membrane (Invitrogen). For membrane blocking and antibody incubations of p70 S6K, Phospho-p70 S6K (Thr389), PathscanI and S6 antibodies (Cell Signalling Technology) a buffer containing 0.2% Tween-20, 5% non-fat dry milk, 5% FBS, in Tris buffered Saline (TBS) were used. For immunoblotting of 4EBP1, Phospho 4EBP1 (Thr37/46), Phospho 4EBP1 (Ser65) (Cell Signalling Technology) and Cyclin D3 (Santa Cruz) a protocol from Cell Signalling Technology were used. Cell Signalling Technology blocking buffer contains 0.1% Tween-20, 5% non fat dry milk in TBS and primary antibody dilution buffer contains 0.1% Tween-20, 5% BSA in TBS. Before adding primary antibody dilution buffer to the membranes, the blots were rinsed briefly in 0.1% Tween-20. All antibody incubations were done overnight at 4° C. overnight. After washing the membranes with 0.1% Tween-20 in TBS, the blots were incubated with horseradish peroxidase conjugated anti-Rabbit IgG (1:1000-1:3000; Amersham Biosciences) at room temperature for 1 hour. Peroxidase activity was detected using the ECL detection system (Amersham Biosciences).

Results

Western blot analyses demonstrate that compounds of general formula (I), such as Compound 3 (lanes 2 and 3), inhibit the phosphorylation of p70S6K and 56 ribosomal protein in MDA-468 cells following 24 hour incubation (FIG. 4). Similar effects are observed with the mTOR inhibitor, rapamycin (lane 5) and the PI3 kinase inhibitor LY294002 (lane 6). AKT phosphorylation on Ser473 is not inhibited by Compound 3 or rapamycin, whereas LY294002 inhibits the phosphorylation of AKT on Ser473. Furthermore, Compound 3 induces a gel mobility shift in 4E-BP1 as shown using both total and thr37/46 phospho-specific anti-4E-BP1 antibodies, indicative of an alteration in the phosphorylation status of 4E-BP1. This is confirmed by the inhibitory effect of Compound 3 on the phosphorylation of ser65 of 4E-BP1. Similar effects are observed with the mTOR inhibitor, rapamycin and the PI3 kinase inhibitor LY294002. In addition, expression of the cell cycle regulatory protein cyclin D3 is reduced by Compound 3, rapamycin and LY294002. These data suggest that mammalian homologue of TOR (mTOR) kinase is active in MDA-468 cells under growth conditions, leading to phosphorylation of mTOR target proteins such as p70S6 kinase (p70S6K) and 4EBP1, and downstream regulation of protein synthesis and cell proliferation via S6 ribosomal protein, eukaryotic translation initiation factor, eIF4, and cyclin D3. Compounds of general formula (I), such as Compound 3, as well as rapamycin and LY294002, inhibit this pathway in MDA-468 cells and might be expected to reduce protein synthesis and cell proliferation.

Compound 3 (lane 2) did not inhibit the phosphorylation of p70S6K, or induce a gel mobility shift in total p70S6K, in MDA-231 cells following 48 hour incubation (FIG. 5). In contrast, rapamycin (lane 5) and LY294002 (lane 6) inhibit the phosphorylation of p70S6K, and induce a gel mobility shift in total p70S6K, following 48 hour incubation in MDA-231 cells. Compound 3, rapamycin and LY294002 all inhibit the phosphorylation of p70S6K and induce a gel mobility shift in total p7056K in MDA-468 cells following 48 hour incubation, demonstrating a cell selective effect of compounds of general formula (I), such as Compound 3.

### Example 5

### Prostate Tumour Xenograft Studies (Human PC3M Cell Line)

The purpose of this study was to evaluate whether compounds of general formula (I), such as Compound 3, inhibit the growth of cancer cells in a xenograft animal model.

Method

Male nude NMRU nu/nu mice weighing 25-45 grams are implanted with PRXF PC3M tumours by subcutaneous implantation in both flanks. Compound 3 (50 & 100 mg) is administered daily by the per-oral (PO) route in an appropriate vehicle (2% DMSO: 5% Tween 80: 93% saline) either alone or in combination with a sub-optimal dose of paclitaxol (10 mg/kg; intravenous; given once/week). Tumor volume is determined once or twice/week for a period of 17 days.

Results

Compound 3 reduces the rate of tumour cell growth when given as a monotherapy (see FIG. 6). Furthermore, additive anti-growth effects are noted in combination with paclitaxol.

### Example 6

### Effect of Compound 3 on Cell Proliferation of Breast, Prostate and Colon Cancer Cell Lines

Methods:

Cell culture: All cell lines except MCF10A are maintained in RPMI medium containing 10% foetal Bovine Serum (FBS) 100 U/ml penicillin, and 100 μg/ml streptomycin. MCF10A is maintained in mammary epithelial growth medium (MEGM) with singlequot addition (BPE, hydrocortisone, hEGF, insulin, gentamicin/amphotericin-B) (Clonetics/Cambrex Bio Science). All cell lines are incubated at 37° C., 5% CO₂, and 95% humidity.

Alamar Blue cell proliferation assay: Cells are plated in black cell culture treated Packard/Perkin Elmer 96-viewplates in 100 μl/well RPMI medium containing 100% FBS, 100 U/ml penicillin, and 100 μg/ml streptomycin. Cell proliferation has been estimated in triplicate for all cell lines in medium containing either 1% FBS or 10% FBS. Cell densities are estimated based on growth during the assay to 80-90% confluency, and are shown in Table 1. The day after plating, the growth medium is changed to either 100 μl/well RPMI containing 1% FBS, 100 U/ml penicillin, 100 μg/ml streptomycin and 25 μg/ml gentamicin or to 100 μl/well RPMI containing 10% FBS, 100 U/ml penicillin, 100 μg/ml streptomycin and 25 μg/ml gentamicin. Compounds are added in 9 point half-log dilution series at concentrations indicated in the graphs. All data based on multiple determinations have been aggregated according to business rules standard to a person skilled in the art. Furthermore, two dilution formats have been used to determine the IC₅₀ values: (1) The standard condition are 9 half-log dilutions starting from 32 μM; and (2) if the compound IC₅₀<100 nM, 9 half-log dilutions starting from 3.2 μM have been used. Briefly, compounds are diluted in compound plates in growth medium containing either 1% FBS or 10% FBS corresponding to the medium in the plates. Compounds are transferred to the cell plates by transfer of 100 μl/well, resulting in a total volume of 200 μl/well containing compound at concentrations indicated in graphs and 0.25% DMSO. Terfenedine is used as a control for maximal cell kill in wells containing 50 μM terfenedine and 0.5% DMSO (Smax). Negative control wells (So) contain medium with 0.25% DMSO.

After compound addition cell plates are incubated undisturbed for 72 hours at 37° C., 5% CO₂, and 95% humidity.

The number of viable cells is estimated using an Alamar Blue assay that measures mitochondrial activity. The medium is decanted and replaced with 150 μl/well RPMI medium without phenol-red containing 100 U/ml penicillin, and 100 μg/ml streptomycin and 10% Alamar Blue. The plates are placed in the incubator at 37° C., 5% CO₂, and 95% humidity for 2 hours. Then, plates are moved to a table and allowed to cool to room temperature without stacking the plates. Alamar Blue signal is read in a fluorescence plate reader using a 590 nm emission filter and a 530 nm excitation filter.

Data handling/calculations: Data are normalised to values from 0% activity (S₀) to 100% activity (Sₘₐₓ). Average values for S₀ and Sₘₐₓ are calculated and used to calculate percent activity (PCTACT) in the assays by the formula: PCTACT=(X_{raw}−Sₘₐₓ)/(S₀−Sₘₐₓ)*100.

Z′-values for assay plates are calculated by:

Z′=1-3*(STDEV(S₀)+STDEV(Sₘₐₓ))/(S₀−Sₘₐₓ). In average Z′˜0.8 and always above 0.6.

Sigmoidal curve fitting is done using Prism using the equation: Y=Bottom+(Top−Bottom)/(1+10ˆ((LogIC₅₀−X)*HillSlope)).

TABLE 1
Cell densities at plating in 96-well plates

| | | | Cells/well |
|---|---|---|---|
| Cell line | Cancer | Cells/well in 1% FBS | in 10% FBS |
| MDA-MB-231 | Breast | 6000 | 4000 |
| MDA-MB-435S | Breast | 10000 | 5000 |
| MDA-MB-453 | Breast | 3000 | 2000 |
| MDA-MB-468 | Breast | 6000 | 4000 |
| SKBr-3 | Breast | 7000 | 6000 |
| BT-474 | Breast | 10000 | 10000 |
| BT-549 | Breast | 6000 | 5000 |
| MCF-7 | Breast | 5000 | 5000 |
| T-47D | Breast | 5000 | 5000 |
| ZR75-1 | Breast | 7000 | 7000 |
| HCC-1954 | Breast | 5000 | 2500 |
| MCF-10A | Normal breast | 18000 | |
| | epithelial cells | (MEGM medium) | |
| PC-3 | Prostate | — | 3000 |
| PC-3/M | Prostate | — | 3000 |
| DU-145 | Prostate | — | 1250 |
| LnCaP | Prostate | — | 8000 |
| Colo205 | Colon | — | 5000 |

Results:

All cell lines are run in cell proliferation in medium containing either 1% serum or 10% serum, both estimations in triplicate. Percent activity (PCTACT) in the assays, equal to percent inhibition of growth, is calculated as described in Methods.

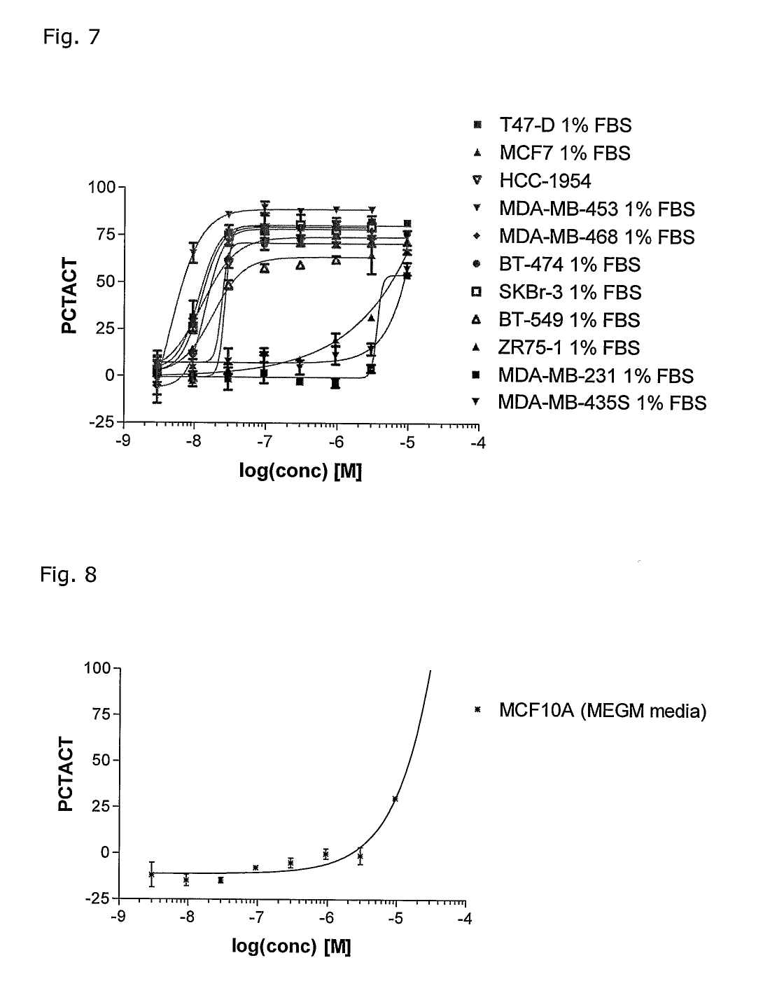

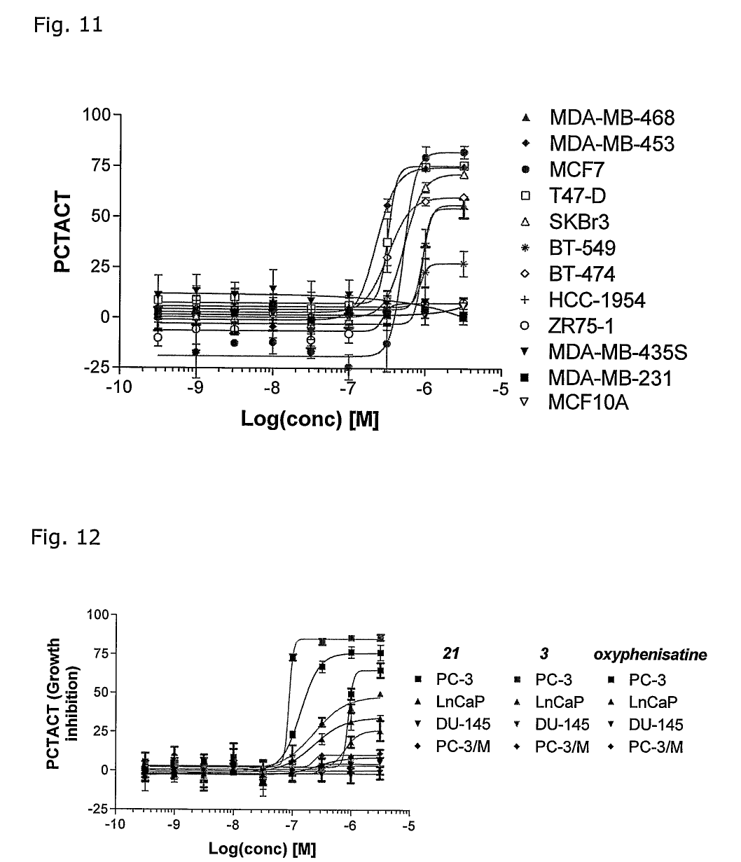

Table 2 summarizes the IC₅₀ values for cell proliferation inhibition of the cell lines. IC₅₀ values refer to the concentration of compound required to inhibit cell proliferation by 50%. Cell proliferation curve fits are shown in FIGS. 7 to 14.

Breast cancer cell lines: A broad panel of breast cancer cell lines have been tested for their sensitivity to Compound 3 as well as Compound 21 and oxyphenisatin. The tested cell lines fall into two very clear categories. 1) Cell lines that are sensitive to Compound 3. Cell proliferation IC₅₀ values range from 0.6 nM to 30 nM when assayed in 1% FBS and between 15 and 80 nM when assayed in 10% FBS. These include the breast cancer cell lines T47-D, MCF-7, MDA-MB-453, MDA-MB-468, BT-474, SKBr-3, BT-549, and HCC-1954 grown under both high (10% FBS) and low (1% FBS) serum conditions. 2) Cell lines that are insensitive to Compound 3 with IC₅₀ values above 3 μM. These include MDA-MB-231, MDA-MB-435S and ZR75-1 grown under both high (10% FBS) and low (1% FBS) serum conditions. The non-transformed breast epithelial cell line, MCF10A, is also insensitive to Compound 3.

Percent activity relative to growth inhibition with 50 μM terfenedine ranged from 60% to 90% growth inhibition. In general the cell lines are more sensitive to the compound under low (1% FBS) serum conditions than under high (10% FBS) serum conditions. The most sensitive breast cancer line is MDA-MB-453.

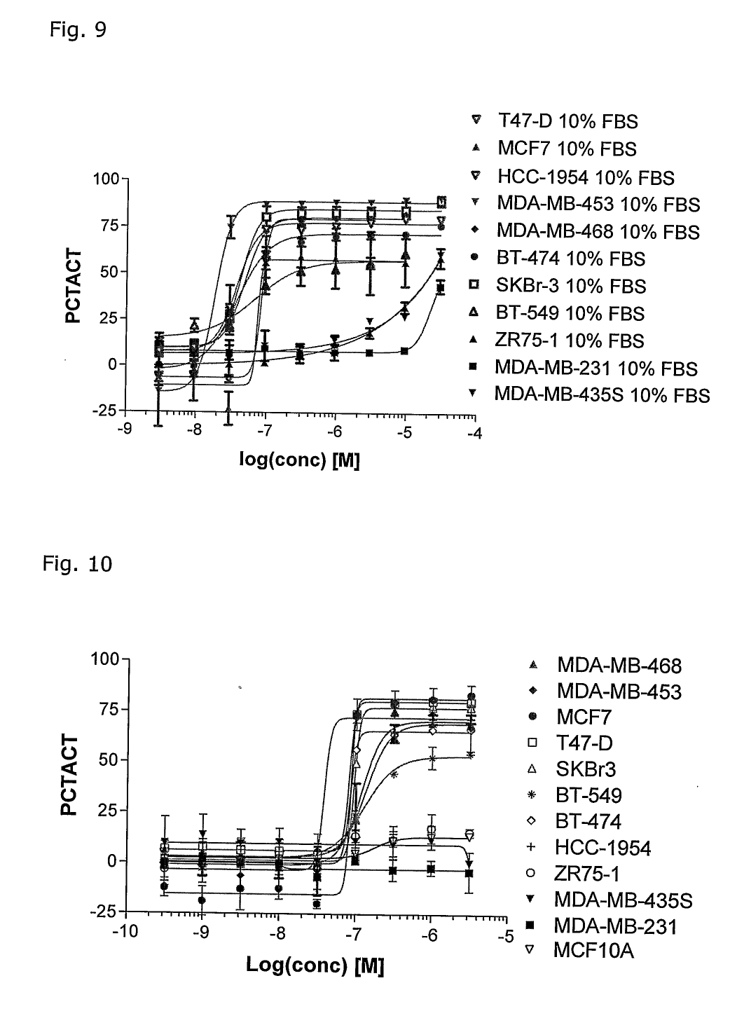

Two other compounds in the series have also been tested, Compound 21 and oxyphenisatine. Both compounds have exactly the same cell line anti-proliferative profile as Compound 3, but are slightly lower in potency (compare FIGS. 9, 10 and 11).

The results are summarized in Table 2 and FIGS. 7-11.

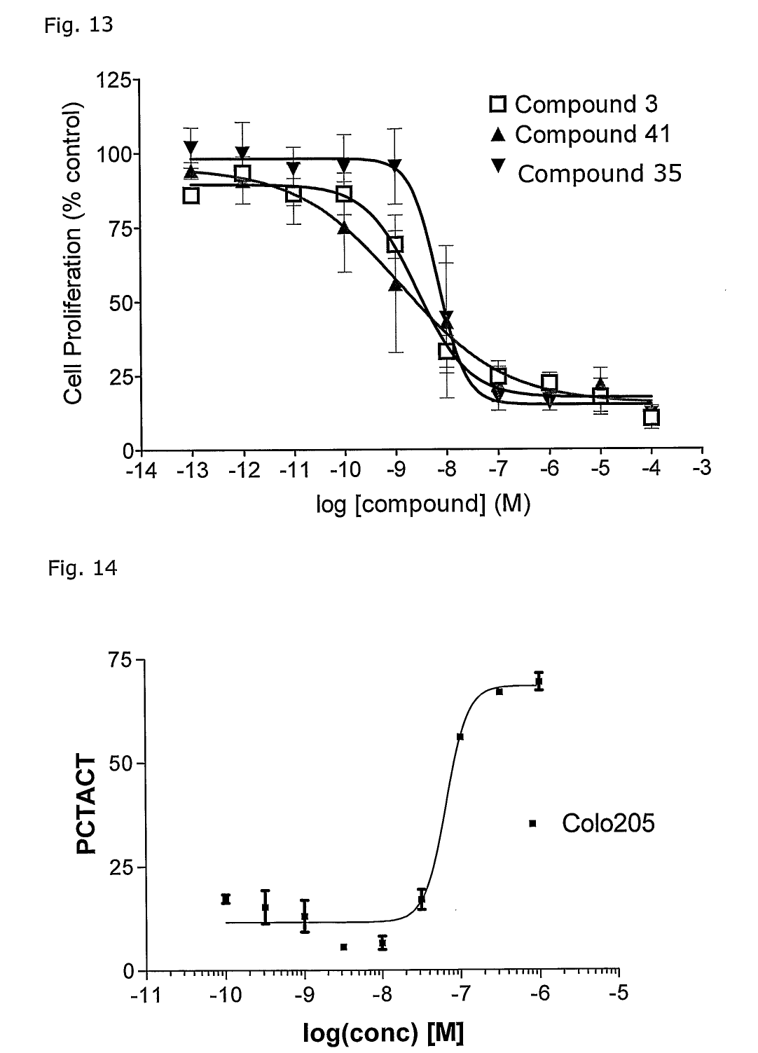

Prostate cancer cell lines: The DU-145, PC-3, PC-3/M and LnCaP prostate cancer cell lines have been tested in cell proliferation assays. PC-3 is highly sensitive to Compound 3, while LnCaP is less sensitive, and PC-3/M and DU-145 are insensitive. Compound 21 and oxyphenisatine have the same cell line sensitivity profile, however, these compounds have lower potency than Compound 3. The results are summarized in Table 2 and FIG. 12. The effect of Compounds 41 and 35 was also compared with Compound 3; both compounds inhibit the proliferation of the PC3 human prostate cancer cell line (FIG. 13).

Colon cancer cell lines: The colon cancer cell line Colo205 has been tested in a cell proliferation assay with Compound 3 resulting an IC₅₀=66 nM. The results are summarized in Table 2 and FIG. 14.

TABLE 2
Summary table of IC₅₀ values for inhibition of cell proliferation.

| | | 3 | | 21 | Oxyphenisatine |
|---|---|---|---|---|---|
| | | 1% FBS | 10% FBS | 10% FBS | 10% FBS |
| Cell line | Cancer | EC50 (nM) | EC50 (nM) | EC50 (nM) | EC50 (nM) |
| T47-D | Breast | 11 | 37 | 83 | 324 |
| MCF7 | Breast | 24 | 74 | 85 | 517 |
| MDA-MB-435S | Breast | >3000 | >3000 | >3000 | >3000 |
| MDA-MB-453 | Breast | 4 | 18 | 38 | 228 |
| MDA-MB-468 | Breast | 14 | 48 | 138 | 935 |
| MDA-MB-231 | Breast | >3000 | >3000 | >3000 | >3000 |
| BT-474 | Breast | 13 | 37 | 85 | 324 |
| SKBr-3 | Breast | 12 | 43 | 95 | 527 |
| BT-549 | Breast | 18 | 68 | 131 | 859 |
| ZR75-1 | Breast | >3000 | >3000 | >3000 | 285 |
| HCC-1954 | Breast | 27 | 84 | 119 | 912 |
| MCF10A | Normal breast | >3000 | | >3000 | >3000 |
| | epithelial | | | | |
| PC-3 | Prostate | — | 87 | 138 | 899 |
| LnCaP | Prostate | — | 235 | 233 | 790 |
| DU-145 | Prostate | — | >3000 | >3000 | >3000 |
| PC-3/M | Prostate | — | >3000 | >3000 | >3000 |
| Colo205 | Colon | — | 66 | — | — |

Notes:
IC₅₀ values are shown in nanomolar concentration. Growth inhibition (PCTACT) lower than 20% is considered insignificant. MCF10A cells are maintained in serum-free MEGM medium. Values are calculated by Prism.

### Example 7

### Xenograft Studies Using MDA-MB-468 Tumours

The purpose of this study was to evaluate whether compounds of general formula (I), such as Compounds 3 and 41, inhibit the growth of tumours derived from MDA-MB-468 breast cancer cells (hormone insensitive human breast cancer cells) in a xenograft animal model.

Method

Nude balb/c mice weighing 25-45 grams are implanted with MDA-MB-468 tumours by subcutaneous implantation in both flanks. Compounds 3 and 41 are administered either daily for 15 days by the per-oral (PO) route (50 & 100 mg) in an appropriate vehicle (2% DMSO: 5% Tween 80: 93% saline) or weekly for 4 weeks by the intravenous (IV) route (25 & 50 mg/kg) in an appropriate vehicle (2% DMSO: 5% Tween 80: 93% saline). Tumour volume is determined once or twice/week.

Results

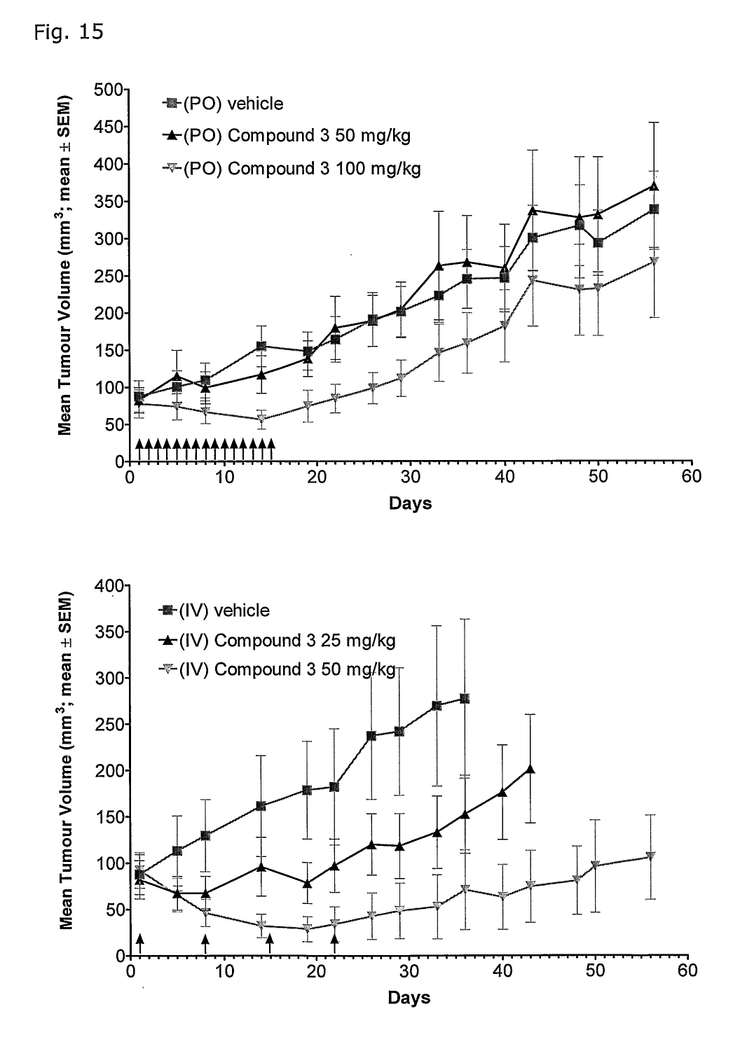

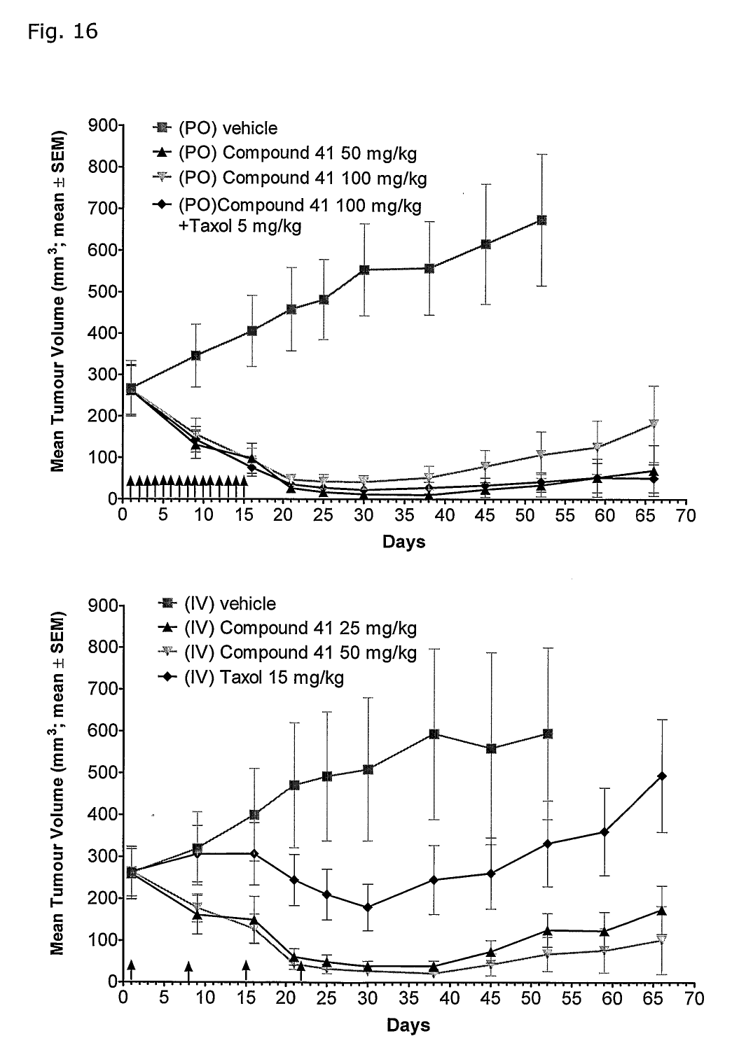

Compound 3 reduces the rate of MDA-MB-468 tumour cell growth in a dose related manner when given as a monotherapy either by the PO or IV route (see FIG. 15). Furthermore, tumour regression is noted using the higher doses of Compound 3. Intravenous dosing with Compound 3 appeared to be more effective than per-oral dosing (FIG. 15). Compound 41 is more effective than Compound 3, inducing a more pronounced tumour regression at all doses tested (FIG. 16). Furthermore, Compound 41 was equally effective by per-oral and intravenous dosing (FIG. 16). Compound 41 also appeared to be more effective than paclitaxel in these studies (FIG. 16).

### Example 8

### Xenograft Studies Using MCF-7 Tumours

The purpose of this study was to evaluate whether compounds of general formula (I), such as Compound 41, inhibit the growth of tumours derived from MCF-7 breast cancer cells (hormone responsive human breast cancer cells) in a xenograft animal model.

Method

Nude balb/c mice weighing 25-45 grams are implanted with MCF-7 tumours by subcutaneous implantation in both flanks. Compounds 3 and 41 are administered either daily for 15 days by the per-oral (PO) route (20 & 100 mg) in an appropriate vehicle (2% DMSO: 5% Tween 80: 93% saline) or weekly for 4 weeks by the intravenous (IV) route (10 & 50 mg/kg) in an appropriate vehicle (2% DMSO: 5% Tween 80: 93% saline). Tumour volume is determined once or twice/week.

Results

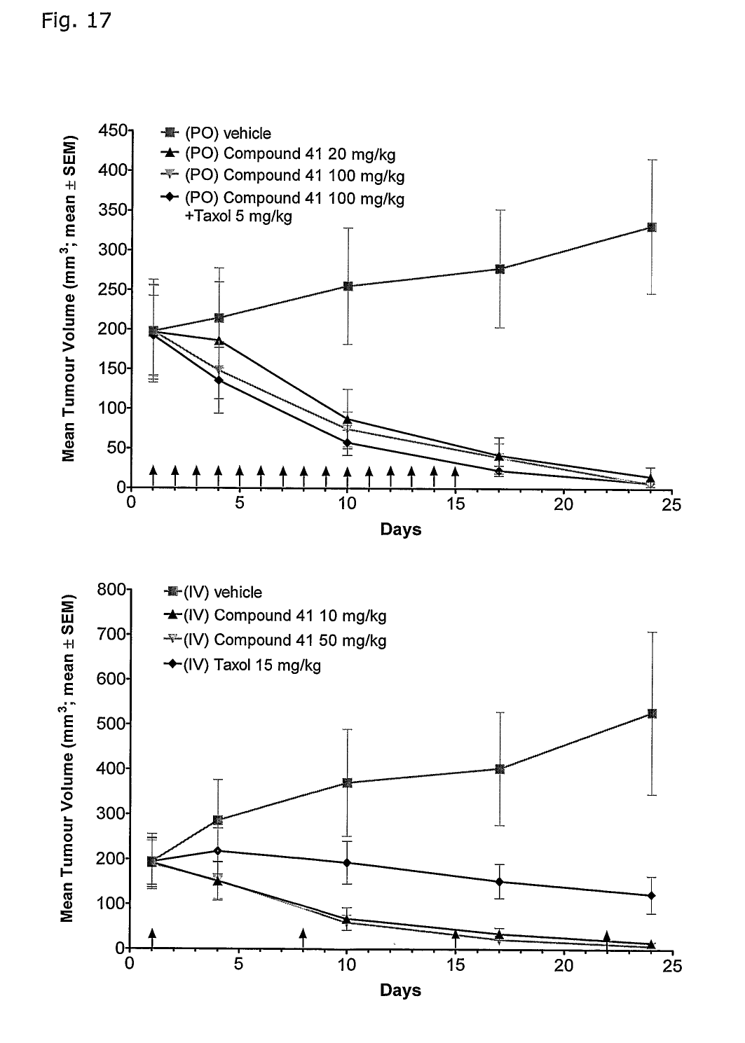

Compound 41 reduces the size of MCF7 tumours when given as a monotherapy either by the PO or IV route (see FIG. 17). Furthermore, tumour regression is noted using all doses tested. The effect of Compound 41 appears to be greater than paclitaxel in this model (FIG. 17). Compound 41 was equally effective by the per-oral and intravenous dosing.

### Example 9

### Activation of Caspase Activity

The purpose of this study was to evaluate whether compounds of general formula (I), such as Compound 3 affect caspase activity as a marker of apoptosis, a form of cell death. Both short-term, medium-term and long-term effects of Compound 3 are assessed by measuring caspase activity at 4, 6 and 22 hours post compound addition.

Method

Human breast cancer cell lines are seeded at 8000 cells/well in 96-well black Packard Viewplates and maintained in RPMI medium containing 10% foetal Bovine Serum (FBS) 100 U/ml penicillin, and 100 μg/ml streptomycin overnight at 37° C., 5% CO₂ in a humidified incubator. Compounds such as Compound 3 are then added to the well and caspase activity is measured at various timepoints using a Caspase activity kit (fluorogenic “Apo-ONE® Homogeneous Caspase-3/7 Assay” kit, #G7791; Promega) according to the manufacturers instructions. Fluorescence intensity (485/535 nm) is measured using on EnVision platereader. Reagent background values (mean of all 8 wells) are subtracted from the experimental wells.

Results

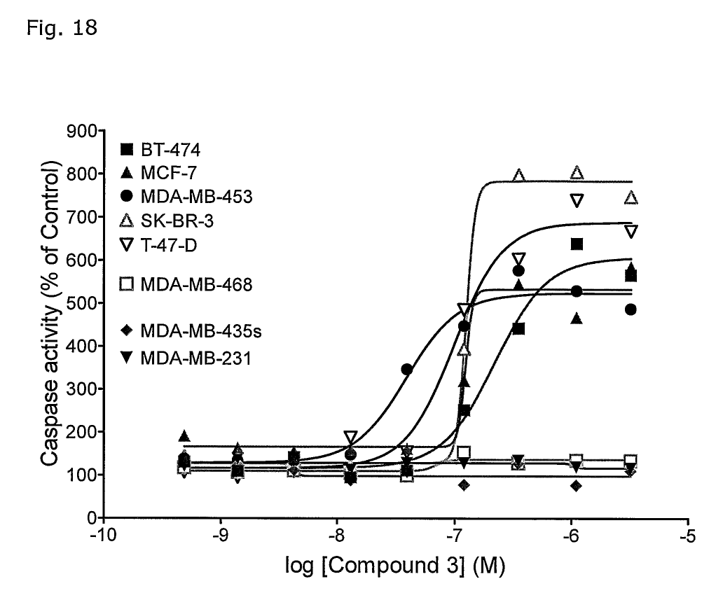

Addition of Compound 3 for 6 hours activates caspase activity in human breast cancer cell lines that are sensitive to the anti-proliferative effects of Compound 3 (FIG. 18), although no activation is noted in MDA-468 cells. Activation of caspase activity is not observed in breast cancer cell lines that are insensitive to the anti-proliferative effect of Compound 3.

These results suggest that compounds of general formula (I), such as Compound 3, may induce apoptotic cell death in certain human breast cancer cell lines.

1: A method of treating a mammal suffering from or being susceptible to cancer, the method comprising administering to the mammal a therapeutically effective amount of a compound of the general formula (I) 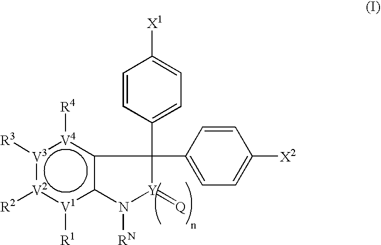
wherein
V¹, V², V³, and V⁴ independently are selected from a carbon atom, a non-quaternary nitrogen atom, an oxygen atom, and a sulfur atom, and where V⁴ further may be selected from a bond, so that —V¹—V²—V³—V⁴— together with the atoms to which V¹ and V⁴ are attached form an aromatic or heteroaromatic ring;
R¹, R², R³, and R⁴, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, aryl, aryloxy, arylcarbonyl, arylamino, heterocyclyl, heterocyclyloxy, heterocyclylamino, heterocyclylcarbonyl, heteroaryl, heteroaryloxy, heteroarylamino, heteroarylcarbonyl, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
R¹, R², R³, and R⁴, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)-aminocarbonyl, amino, C₁₋₆-alkylcarbonylamino, mono- and di(C₁₋₆-alkyl)amino, C₁₋₆-alkylsulphonyl, C₁₋₆-alkylsulphinyl, aryl, aryloxy, arylcarbonyl, arylamino, heterocyclyl, heterocyclyloxy, heterocyclylcarbonyl, heterocyclylamino, heteroaryl, heteroaryloxy, heteroarylcarbonyl, and heteroarylamino; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
or R¹ and R² together with the carbon atoms to which they are attached form a ring;
X¹ and X² are independently selected from halogen, hydroxy, optionally substituted C₁₋₆-alkoxy, optionally substituted C₁₋₆-alkylcarbonyloxy, amino, mono- and di(C₁₋₆-alkyl)amino, C₁₋₆-alkylcarbonylamino, C₁₋₆-alkylsulphonylamino, mono- and di(C₁₋₆-alkyl)aminocarbonylamino, C₁₋₆-alkanoyloxy, mercapto, optionally substituted C₁₋₆-alkylthio, C₁₋₆-alkylsulfonyl, mono- and di(C₁₋₆-alkyl)aminosulfonyl, aryloxy, arylamino, heterocyclyloxy, heterocyclylamino, heteroaryloxy and heteroarylamino, where any C₁₋₆-alkyl as an amino or sulphur substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s), and wherein any aryl, heterocyclyl and heteroaryl may be optionally substituted;
>Y(=Q)ₙ is selected from >C═O, >C═S, >S═O and >S(═O)₂; and
R^{N} is selected from the group consisting of hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s); and
pharmaceutically acceptable salts and prodrugs thereof.

2: The method according to claim 1, wherein R¹, R², R³ and R⁴ are not all hydrogen.

3: The method according to claim 1, wherein the ring is selected from a benzene ring and a pyridine ring where the nitrogen atom represents V³.

4: The method according to claim 1, wherein R¹ is selected from hydrogen, halogen, C₁₋₆-alkyl, trifluoromethyl and C₁₋₆-alkoxy, when V¹ is a carbon atom.

5: The method according to claim 1, wherein R² is selected from hydrogen, halogen, optionally substituted aryl, optionally substituted aryloxy, and optionally substituted heteroaryl, when V² is a carbon atom.

6: The method according to claim 1, wherein R³ is selected from hydrogen, optionally substituted C₁₋₆-alkoxy, halogen, cyano, optionally substituted aryl, optionally substituted aryloxy, optionally substituted heteroaryl, amino, C₁₋₆-alkylcarbonylamino, C₁₋₆-alkylsulphonylamino, and mono- and di(C₁₋₆-alkyl)aminosulfonyl, when V³ is a carbon atom.

7: The method according to claim 1, wherein R⁴ is hydrogen, when V⁴ is a carbon atom.

8: The method according to claim 1, wherein X¹ and X² independently are selected from hydroxy, OAc, NH₂, NMe₂, NHAc, NHSO₂Me and NHCONMe₂.

9-10. (canceled)

11: The method according to claim 1, wherein V¹, V², V³, V⁴ all are a carbon atom, >Y(=Q)ₙ is >C═O, and R^{N} is hydrogen.

12-20. (canceled)

21: The method according to claim 11, wherein R¹ is selected from fluoro, chloro, bromo, C₁₋₄-alkyl, trifluoromethyl, C₁₋₄-alkoxy, and dimethylaminocarbonyl.

22. (canceled)

23: The method according to claim 11, wherein R¹ is selected from halogen, C₁₋₄-alkyl, trifluoromethyl, C₁₋₄-alkoxy, and dimethylaminocarbonyl, R² is selected from hydrogen and halogen, and R³ is selected from hydrogen, halogen, C₁₋₄-alkyl, and amino; where R² and R³ are not both hydrogen.

24: A method of treating a mammal suffering from or being susceptible to cancer, the method comprising administering to the mammal a therapeutically effective amount of a 3,3-diphenyl-1,3-dihydro-indol-2-one type compound of the formula (IIa) 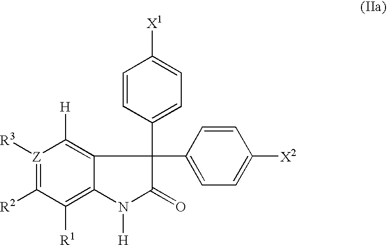
wherein
R¹ is selected from hydrogen, halogen, C₁₋₆-alkyl, trifluoromethyl and C₁₋₆-alkoxy;
R² is selected from hydrogen, halogen, optionally substituted aryl, optionally substituted aryloxy, and optionally substituted heteroaryl;
R³ is selected from hydrogen, optionally substituted C₁₋₆-alkoxy, halogen, cyano, and optionally substituted aryl, optionally substituted aryloxy, optionally substituted heteroaryl, amino, C₁₋₆-alkylcarbonylamino, C₁₋₆-alkylsulphonylamino, and mono- and di(C₁₋₆-alkyl)aminosulfonyl;
Z is CH or N; and
X¹ and X² are independently selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; and
pharmaceutically acceptable salts and prodrugs thereof.

25: A method of treating a mammal suffering from or being susceptible to cancer, the method comprising administering to the mammal a therapeutically effective amount of a 3,3-diphenyl-1,3-dihydro-indol-2-one type compound of the formula (IIb) 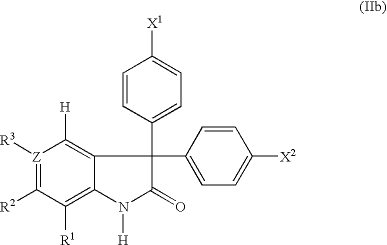
wherein
R¹, R², and R³, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s); and
R¹, R², and R³, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)-aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkyl-carbonylamino, C₁₋₆-alkylaminocarbonyl, or halogen(s), and wherein any aryl, heterocyclyl and heteroaryl may be optionally substituted;
or wherein R¹ and R² together with the carbon and/or nitrogen atoms to which they are attached form a heterocyclic ring, a heteroaromatic ring, an aromatic ring or a carbocyclic ring;
Z is CH or N; and
X¹ and X² are independently selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; and
pharmaceutically acceptable salts and prodrugs thereof.

26: A method of treating a mammal suffering from or being susceptible to cancer, the method comprising administering to the mammal a therapeutically effective amount of a 3,3-diphenyl-1,3-dihydro-indol-2-one type compound of the formula (IIc) 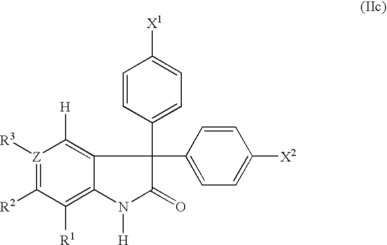
wherein
R¹ is selected from hydrogen, halogen, C₁₋₆-alkyl, trifluoromethyl and C₁₋₆-alkoxy;
R² is selected from hydrogen, halogen, optionally substituted aryl, optionally substituted aryloxy, and optionally substituted heteroaryl;
R³ is selected from hydrogen, optionally substituted C₁₋₆-alkoxy, halogen, cyano, and optionally substituted aryl, optionally substituted aryloxy, optionally substituted heteroaryl, amino, C₁₋₆-alkyl-carbonylamino, C₁₋₆-alkylsulphonylamino, and mono- and di(C₁₋₆-alkyl)aminosulfonyl;
Z is CH or N; and
one of X¹ and X² is selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; and the other of X¹ and X² is selected from optionally substituted C₁₋₆-alkyl, optionally substituted C₂₋₆-alkenyl, carboxy, optionally substituted C₁₋₆-alkoxycarbonyl, optionally substituted C₁₋₆-alkylcarbonyl, formyl, carbamoyl, mono- and di(C₁₋₆-alkyl)aminocarbonyl, cyano, aryl, arylcarbonyl, heterocyclyl, heterocyclylcarbonyl, heteroaryl, heteroarylcarbonyl, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkyl-carbonylamino, C₁₋₆-alkylaminocarbonyl, or halogen(s), and wherein any aryl, heterocyclyl and heteroaryl may be optionally substituted; and
pharmaceutically acceptable salts and prodrugs thereof.

27: A method of treating a mammal suffering from or being susceptible to cancer, the method comprising administering to the mammal a therapeutically effective amount of a 3,3-diphenyl-1,3-dihydro-indol-2-one type compound of the formula (IId) 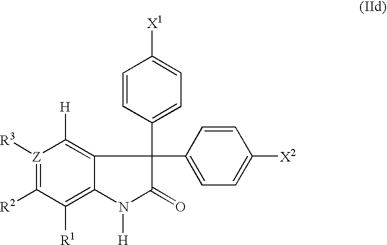
wherein
R¹, R², and R³, when attached to a carbon atom, independently are selected from hydrogen, optionally substituted C₁₋₆-alkyl, optionally substituted C₂₋₆-alkenyl, hydroxy, optionally substituted C₁₋₆-alkoxy, optionally substituted C₂₋₆-alkenyloxy, carboxy, optionally substituted C₁₋₆-alkoxycarbonyl, optionally substituted C₁₋₆-alkylcarbonyl, optionally substituted C₁₋₆-alkylcarbonyloxy, formyl, amino, mono- and di(C₁₋₆-alkyl)amino, carbamoyl, mono- and di(C₁₋₆-alkyl)aminocarbonyl, C₁₋₆-alkylcarbonylamino, C₁₋₆-alkylsulphonylamino, cyano, carbamido, mono- and di(C₁₋₆-alkyl)aminocarbonylamino, C₁₋₆-alkanoyloxy, C₁₋₆-alkylsulphonyl, C₁₋₆-alkylsulphinyl, aminosulfonyl, mono- and di(C₁₋₆-alkyl)aminosulfonyl, nitro, optionally substituted C₁₋₆-alkylthio, and halogen, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen(s); and
R¹, R², and R³, when attached to a nitrogen atom, independently are selected from hydrogen, optionally substituted C₁₋₆-alkyl, hydroxy, optionally substituted C₁₋₆-alkoxy, optionally substituted C₁₋₆-alkoxycarbonyl, optionally substituted C₁₋₆-alkylcarbonyl, formyl, mono- and di(C₁₋₆-alkyl)-aminocarbonyl, amino, C₁₋₆-alkylcarbonylamino, mono- and di(C₁₋₆-alkyl)amino, C₁₋₆-alkylsulphonyl, and C₁₋₆-alkylsulphinyl; where any C₁₋₆-alkyl as an amino substituent is optionally substituted with hydroxy, C₁₋₆-alkoxy, amino, mono- and di(C₁₋₆-alkyl)amino, carboxy, C₁₋₆-alkyl-carbonylamino, C₁₋₆-alkylaminocarbonyl, or halogen(s), and wherein any aryl, heterocyclyl and heteroaryl may be optionally substituted;
or wherein R¹ and R² together with the carbon and/or nitrogen atoms to which they are attached form a heterocyclic ring, a heteroaromatic ring, an aromatic ring or a carbocyclic ring;
Z is CH or N; and
one of X¹ and X² is selected from halogen, OR⁶, OCOR⁵, N(R⁶)₂, NHCOR⁵, NHSO₂R⁵, and NHCON(R⁶)₂, wherein R⁵ is selected from C₁₋₆-alkyl, optionally substituted aryl and optionally substituted heteroaryl, and each R⁶ independently is selected from hydrogen, C₁₋₆-alkyl, optionally substituted aryl and optionally substituted heteroaryl; and the other of X¹ and X² is selected from optionally substituted C₁₋₆-alkyl, optionally substituted C₂₋₆-alkenyl, carboxy, optionally substituted C₁₋₆-alkoxycarbonyl, optionally substituted C₁₋₆-alkylcarbonyl, formyl, carbamoyl, mono- and di(C₁₋₆-alkyl)aminocarbonyl, cyano, aryl, arylcarbonyl, heterocyclyl, heterocyclylcarbonyl, heteroaryl, heteroarylcarbonyl, where any C₁₋₆-alkyl as an amino substituent is optionally substituted with hydroxy, C₁₋₆-alkoxy, amino, mono- and di(C₁₋₆-alkyl)amino, carboxy, C₁₋₆-alkyl-carbonylamino, C₁₋₆-alkylaminocarbonyl, or halogen(s), and wherein any aryl, heterocyclyl and heteroaryl may be optionally substituted; and
pharmaceutically acceptable salts and prodrugs thereof.

28. The method according to claim 1, wherein the compound is selected from Items 1 to 225 listed below:
1 5-Amino-6-chloro-3,3-bis-(4-hydroxy-phenyl)-7-methyl-1,3-dihydro-indol-2-one
2 5-Chloro-3,3-bis-(4-hydroxy-phenyl)-7-methyl-1,3-dihydro-indol-2-one
3 5-Fluoro-3,3-bis-(4-hydroxy-phenyl)-1,3-dihydro-indol-2-one
4 3,3-Bis-(4-hydroxy-phenyl)-5-nitro-1,3-dihydro-indol-2-one
5 3,3-Bis-(4-hydroxy-phenyl)-7-methyl-1,3-dihydro-pyrrolo[3,2-c]pyridin-2-one
6 6-Bromo-3,3-bis-(4-hydroxy-phenyl)-1,3-dihydro-pyrrolo[3,2-c]pyridin-2-one
7 6-Bromo-3 3-bis-(4-hydroxy-phenyl)-7-methyl-1,3-dihydro-pyrrolo[3,2-c]pyridin-2-one
8 6-Bromo-3,3-bis-(4-hydroxy-phenyl)-5,7-dimethyl-1,3-dihydro-indol-2-one
9 6-Bromo-3,3-bis-(4-hydroxy-phenyl)-7-methyl-2-oxo-2,3-dihydro-1H-indole-5-carbonitrile
10 6-Bromo-3,3-bis-(4-hydroxy-phenyl)-5-methoxy-7-methyl-1,3-dihydro-indol-2-one
11 6-Bromo-3,3-bis-(4-hydroxy-phenyl)-7-methoxy-1,3-dihydro-pyrrolo[3,2-c]pyridin-2-one;
12 6-Bromo-7-ethyl-3,3-bis-(4-hydroxy-phenyl)-1,3-dihydro-pyrrolo[3,2-c]pyridin-2-one
13 6-Bromo-7-ethyl-3,3-bis-(4-hydroxy-phenyl)-5-methyl-1,3-dihydro-indol-2-one
14 6-Bromo-5-ethyl-3,3-bis-(4-hydroxy-phenyl)-7-methyl-1,3-dihydro-indol-2-one
15 6-Bromo-7-ethyl-3,3-bis-(4-hydroxy-phenyl)-2-oxo-2,3-dihydro-1H-indole-5-carbonitrile
16 6-Bromo-7-ethyl-3,3-bis-(4-hydroxy-phenyl)-5-methoxy-1,3-dihydro-indol-2-one
17 6-Chloro-3,3-bis-(4-hydroxy-phenyl)-1,3-dihydro-pyrrolo[3,2-c]pyridin-2-one
18 6-Chloro-3,3-bis-(4-hydroxy-phenyl)-7-methyl-1,3-dihydro-pyrrolo[3,2-c]pyridin-2-one
19 6-Chloro-3,3-bis-(4-hydroxy-phenyl)-5,7-dimethyl-1,3-dihydro-indol-2-one
20 6-Chloro-3,3-bis-(4-hydroxy-phenyl)-7-methyl-2-oxo-2,3-dihydro-1H-indole-5-carbonitrile
21 6-Chloro-3,3-bis-(4-hydroxy-phenyl)-5-methoxy-7-methyl-1,3-dihydro-indol-2-one
22 6-Chloro-3,3-bis-(4-hydroxy-phenyl)-7-methoxy-1,3-dihydro-pyrrolo[3,2-c]pyridin-2-one
23 6-Chloro-7-ethyl-3,3-bis-(4-hydroxy-phenyl)-1,3-dihydro-pyrrolo[3,2-c]pyridin-2-one
24 6-Chloro-7-ethyl-3,3-bis-(4-hydroxy-phenyl)-5-methyl-1,3-dihydro-indol-2-one
25 6-Chloro-5-ethyl-3,3-bis-(4-hydroxy-phenyl)-7-methyl-1,3-dihydro-indol-2-one
26 6-Chloro-7-ethyl-3,3-bis-(4-hydroxy-phenyl)-2-oxo-2,3-dihydro-1H-indole-5-carbonitrile
27 6-Chloro-7-ethyl-3,3-bis-(4-hydroxy-phenyl)-5-methoxy-1,3-dihydro-indol-2-one
28 6-Chloro-3,3-bis-(4-hydroxy-phenyl)-5-methyl-7-methoxy-1,3-dihydro-indol-2-one;
29 6-Chloro-3,3-bis-(4-hydroxy-phenyl)-7-methoxy-2-oxo-2,3-dihydro-1H-indole-5-carbonitrile;
30 6-Chloro-3,3-bis-(4-hydroxy-phenyl)-7-methoxy-1,3-dihydro-pyrrolo[3,2-c]pyridin-2-one;
31 6-Chloro-3,3-bis-(4-hydroxy-phenyl)-7-methoxy-5-methyl-1,3-dihydro-indol-2-one;
32 6-Chloro-5-ethyl-3,3-bis-(4-hydroxy-phenyl)-7-methoxy-1,3-dihydro-indol-2-one;
33 6-Chloro-3,3-bis-(4-hydroxy-phenyl)-5,7-dimethoxy-1,3-dihydro-indol-2-one;
34 N-{4-[3-(4-Acetylamino-phenyl)-5-chloro-7-methyl-2-oxo-2,3-dihydro-1H-indol-3-yl]-phenyl}-acetamide;
35 N-{4-[5-Chloro-3-(4-methanesulfonylamino-phenyl)-7-methyl-2-oxo-2,3-dihydro-1H-indol-3-yl]-phenyl}-methanesulfonamide
36 N-{4-[3-(4-Acetylamino-phenyl)-6-chloro-7-methyl-2-oxo-2,3-dihydro-1H-indol-3-yl]-phenyl}-acetamide;
37 N-{4-[6-Chloro-3-(4-methanesulfonylamino-phenyl)-7-methyl-2-oxo-2,3-dihydro-1H-indol-3-yl]-phenyl}-methanesulfonamide;
38 N-{4-[3-(4-Acetylamino-phenyl)-5-chloro-7-methoxy-2-oxo-2,3-dihydro-1H-indol-3-yl]-phenyl}-acetamide;
39 N-{4-[5-Chloro-3-(4-methanesulfonylamino-phenyl)-7-methoxy-2-oxo-2,3-dihydro-1H-indol-3-yl]-phenyl}-methanesulfonamide;
40 N-{4-[3-(4-Acetylamino-phenyl)-6-chloro-7-methoxy-2-oxo-2,3-dihydro-1H-indo-3-yl]-phenyl}-acetamide; and
41 N-{4-[6-Chloro-3-(4-methanesulfonylamino-phenyl)-7-methoxy-2-oxo-2,3-dihydro-1H-indol-3-yl]-phenyl}-methanesulfonamide
42 2-Chloro-6,6-bis-(4-hydroxy-phenyl)-3-methyl-4,6-dihydro-3H-pyrrolo[2,3-d]imidazol-5-one
43 Acetic acid 4-[6-(4-acetoxy-phenyl)-2-chloro-3-methyl-5-oxo-3,4,5,6-tetrahydro-pyrrolo[2,3-d]imidazol-6-yl-phenyl ester
44 6,6-Bis-(4-amino-phenyl)-2-chloro-3-methyl-4,6-dihydro-3H-pyrrolo[2,3-d]imidazol-5-one
45 2-Chloro-6,6-bis-(4-dimethylamino-phenyl)-3-methyl-4,6-dihydro-3H-pyrrolo[2,3-d]imidazol-5-one
46 N-{4-[6-(4-Acetylamino-phenyl)-2-chloro-3-methyl-5-oxo-3,4,5,6-tetrahydro-pyrrolo[2,3-d]imidazol-6-yl]-phenyl}-acetamide
47 N-{4-[2-Chloro-6-(4-methanesulfonylamino-phenyl)-3-methyl-5-oxo-3,4,5,6-tetrahydro-pyrrolo[2,3-d]imidazol-6-yl]-phenyl}-methanesulfonamide
48 4,4-Bis-(4-hydroxy-phenyl)-1-methyl-4,6-dihydro-1H-pyrrolo[2,3-c]pyrazol-5-one
49 Acetic acid 4-[4-(4-acetoxy-phenyl)-1-methyl-5-oxo-1,4,5,6-tetrahydro-pyrrolo[2,3-c]pyazol-4-yl]-phenyl ester
50 4,4-Bis-(4-amino-phenyl)-1-methyl-4,6-dihydro-1H-pyrrolo[2,3-c]pyrazol-5-one
51 N-{4-[4-(4-Methanesulfonylamino-phenyl)-1-methyl-5-oxo-1,4,5,6-tetrahydro-pyrrolo[2,3-c]pyrazol-4-yl}-phenyl]-methanesulfonamide
52 4,4-Bis-(4-dimethylamino-phenyl)-1-methyl-4,6-dihydro-1H-pyrrolo[2,3-c]pyrazol-5-one
53 N-{4-[4-(4-Acetylamino-phenyl)-1-methyl-5-oxo-1,4,5,6-tetrahydro-pyrrolo[2,3-c]pyrazol-4-yl]-phenyl}-acetamide
54 4,4-Bis-(4-hydroxy-phenyl)-2-methyl-2,6-dihydro-4H-pyrrolo[2,3-c]pyrazol-5-one
55 Acetic acid 4-[4-(4-acetoxy-phenyl)-2-methyl-5-oxo-2,4,5,6-tetrahydro-pyrrolo[2,3-c]pyrazol-4-yl]-phenyl ester
56 4,4-Bis-(4-amino-phenyl)-2-methyl-2,6-dihydro-4H-pyrrolo[2,3-c]pyrazol-5-one
57 4,4-Bis-(4-dimethylamino-phenyl)-2-methyl-2,6-dihydro-4H-pyrrolo[2,3-c]pyrazol-5-one
58 N-{4-[4-(4-Acetylamino-phenyl)-2-methyl-5-oxo-2,4,5,6-tetrahydro-pyrrolo[2,3-c]pyrazol-4yl]-phenyl}-acetamide
59 N-{4-[4-(4-Methanesulfonylamino-phenyl)-2-methyl-5-oxo-2,4,5,6-tetrahydro-pyrrolo[2,3c]pyrazol-[4-yl]-phenyl}-methanesulfonamide
60 4,4-Bis-(4-hydroxy-phenyl)-4,6-dihydro-thieno[2,3-b]pyrrol-5-one
61 Acetic acid 4-[4-(4-acetoxy-phenyl)-5-oxo-5,6-dihydro-4H-thieno[2,3-b]pyrrol-4-yl]-phenyl ester
62 4,4-Bis-(4-amino-phenyl)-4,6-dihydro-thieno[2,3]pyrrol-5-one
63 4,4-Bis-(4-dimethylamino-phenyl)-4,6-dihydro-thieno[2,3-b]pyrrol-5-one
64 N-{4-[4-(4-Acetylamino-phenyl)-5-oxo-5 6-dihydro-4H-thieno[2,3-b]pyrrol-4-yl]-phenyl}-acetamide
65 N-{4-[4-(4-Methanesulfonylamino-phenyl)-5-oxo-5,6-dihydro-4H-thieno[2-b]pyrrol-4-yl]-phenyl}-methanesulfonamide
66 2-Chloro-4,4-bis-(4-hydroxy-phenyl)-4,6-dihydro-thieno[2,3-b]-pyrrol-5-one
67 Acetic acid 4-[4-(4-acetoxy-phenyl)-2-chloro-5-oxo-5,6-dihydro-4H-thieno[2,3-b]pyrrol-4-yl]-phenyl ester
68 4,4-Bis-(4-amino-phenyl)-2-chloro-4,6-dihydro-thieno[2,3-b]pyrrol-5-one
69 2-Chloro-4,4-bis-(4-dimethylamino-phenyl)-4,6-dihydro-thieno[2,3-b]pyrrol-5-one
70 N-{4-[4-(4-Acetylamino-phenyl)-2-chloro-5-oxo-5,6-dihydro-4H-thieno[2,3-b]pyrrol-4-yl]-phenyl}-acetamide
71 N-{4-[2-Chloro-4-(4-methanesulfonylamino-phenyl)-5-oxo-5,6-dihydro-4H-thieno[2,3b]pyrrol-4-yl]-phenyl}-methanesulfonamide
72 4,4-Bis-(4-hydroxy-phenyl)-4,6-dihydro-furo[2,3-b]pyrrol-5-one
73 Acetic acid 4-[4-(4-acetoxy-phenyl)-5-oxo-5,6-dihydro-4H-furo[2,3-b]pyrrol-4-yl]-phenyl
74 4,4-Bis-(4-amino-phenyl)-4,6-dihydro-furo[2,3-b]pyrrol-5-one
75 4,4-Bis-(4-dimethylamino-phenyl)-4,6-dihydro-furo[2,3-b]pyrrol-5-one
76 N-{4-[4-(4-Acetylamino-phenyl)-5-oxo-5 6-dihydro-4H-furo[2,3-b]pyrrol-4-yl]-phenyl}-acetamide
77 N-{-4-[4-(4-Methanesulfonylamino-phenyl)-5-oxo-5,6-dihydro-4H-furo[2,3-b]pyrrol-4]-phenyl}-methanesulfonamide
78 2-Chloro-4,4-bis-(4-hydroxy-phenyl)-4,6-dihydro-furo[2,3-b]pyrrol-5-one
79 Acetic acid 4-[4-(4-acetoxy-phenyl)-2-chloro-5-oxo-5,6-dihydro-4H-furo[2,3-b]pyrrol-4-yl]-phenyl ester
80 4,4-Bis-(4-amino-phenyl)-2-chloro-4,6-dihydro-furo[2,3-b]pyrrol-5-one
81 2-Chloro-4,4-bis-(4-dimethylamino-phenyl)-4,6-dihydro-furo[2,3-b]pyrrol-5-one
82 N-{4-[4-(4-Acetylamino-phenyl)-2-chloro-5-oxo-5,6-dihydro-4H-furo[2,3-b]pyrrol-4-yl]-phenyl}-acetamide
83 N-{4-[2-Chloro-4-(4-methanesulfonylamino-phenyl)-5-oxo-5,6-dihydro-4H-furo[2,3-b]pyrrol-4-yl]-phenyl}-methanesulfonamide
84 3,3-Bis-(4-hydroxy-phenyl)-6-methyl-3,8-dihydro-1H-1,8-diaza-as-indacen-2-one
85 Acetic acid 4-[3-(4-acetoxy-phenyl)-6-methyl-2-oxo-1,2,3,8-tetrahydro-[1,8-diaza-as-indacen-3yl]-phenyl ester
86 3,3-Bis-(4-amino-phenyl)-6-methyl-3,8-dihydro-1H-1,8-diaza-as-indacen-2-one
87 3,3-Bis-(4-dimethylamino-phenyl)-6-methyl-3,8-dihydro-1H-1,8-diaza-as-indacen-2-one
88 N-{4-[3-(4-Acetylamino-phenyl)-6-methyl-2-oxo-1,2,3,8-tetrahydro-1,8-diaza-as-indacen-3-phenyl}-acetamide
89 N-{4-[3-(4-Methanesulfonylamino-phenyl)-6-methyl-2-oxo-1,2,3,8-tetrahydro-1,8-diaza-as-indacen-3-yl]-phenyl}-methanesulfonamide
90 3,3-Bis-(4-hydroxy-phenyl)-1,3-dihydro-benzo[g]indol-2-one
91 Acetic acid 4-[3-(4-acetoxy-phenyl)-2-oxo-2,3-dihydro-1H-benzo[g]indol-3-yl]-phenyl ester
92 3,3-Bis-(4-amino-phenyl)-1,3-dihydro-benzo[g]indol-2-one
93 3,3-Bis-(4-dimethylamino-phenyl)-1,3-dihydro-benzo[g]indol-2-one
94 N-{4-[3-(4-Acetylamino-phenyl)-2-oxo-2,3-dihydro-1H-benzo[g]indol-3-yl]-phenyl}-acetamide
95 N-{4-[3-(4-Methanesulfonylamino-phenyl)-2-oxo-2,3-dihydro-1H-benzo[g]indol-3-yl]-phenyl}-methanesulfonamide
96 1-Amino-6-chloro-3,3-bis-(4-hydroxy-phenyl)-7-methyl-1,3-dihydro-indol-2-one
97 Acetic acid 4-[3-(4-acetoxy-phenyl)-1-amino-6-chloro-7-methyl-2-oxo-2,3-dihydro-1H-indol-]-phenyl ester
98 N-{4-[3-(4-Acetylamino-phenyl)-1-amino-6-chloro-7-methyl-2-oxo-2,3-dihydro-1H-indol-3yl]-phenyl}-acetamide
99 N-{4-[1-Amino-6-chloro-3-(4-methanesulfonylamino-phenyl)-7-methyl-2-oxo-2,3-dihydro-indol-3-yl]-phenyl}-methanesulfonamide
100 Acetic acid 4-[3-(4-acetoxy-phenyl)-1-acetylamino-6-chloro-7-methyl-2-oxo-2,3-dihydro-1H-indol-3-yl]-phenyl ester
101 N-[3,3-Bis-(4-amino-phenyl)-6-chloro-7-methyl-2-oxo-2,3-dihydro-indol-1-yl]-acetamide
102 N-[6-Chloro-3,3-bis-(4-dimethylamino-phenyl)-7-methyl-2-oxo-2,3-dihydro-indol-1-yl]-acetamide
103 N-[3,3-Bis-(4-acetylamino-phenyl)-6-chloro-7-methyl-2-oxo-2,3-dihydro-indol-1-yl]-acetamide
104 N-[6-Chloro-3,3-bis-(4-methanesulfonylamino-phenyl)-7-methyl-2-oxo-2,3-dihydro-indol-1-yl]-acetamide
105 6-Chloro-3,3-bis-(4-hydroxy-phenyl)-7-methyl-1,3-dihydro-indole-2-thione
106 Acetic acid 4-[3-(4-acetoxy-phenyl)-6-chloro-7-methyl-2-thioxo-2,3-dihydro-1H-indol-3-yl]-phenyl ester
107 3,3-Bis-(4-amino-phenyl)-6-chloro-7-methyl-1,3-dihydro-indole-2-thione
108 6-Chloro-3,3-bis-(4-dimethylamino-phenyl)-7-methyl-1,3-dihydro-indole-2-thione
109 N-{4-[3-(4-Acetylamino-phenyl)-6-chloro-7-methyl-2-thioxo-2,3-dihydro-1H-indol-3-yl]-phenyl}-acetamide
110 Methanesulfonic acid 4-[6-chloro-3-(4-methanesulfonyloxy-phenyl)-7-methyl-2-thioxo-2,3-dihydro-1H-indol-3-yl]-phenyl ester
111 Acetic acid 4-[4-(4-acetoxy-phenyl)-2-chloro-5-thioxo-5,6-dihydro-4H-thieno[2,3-b]pyrrol-4-yl]-phenyl ester
112 Acetic acid 4-[4-(4-acetoxy-phenyl)-2-chloro-5-thioxo-5,6-dihydro-4H-furo[2,3-b]pyrrol-4-yl]-phenyl ester
113 6,6-Bis-(4-amino-phenyl)-2-chloro-3-methyl-4,6-dihydro-thieno[3,2-b]pyrrole-5-thione
114 2-Chloro-6,6-bis-(4-dimethylamino-phenyl)-3-methyl-4,6-dihydro-3H-pyrrolo[2,3-d]imidazole-5-thione
115 N-{4-[6-(4-Acetylamino-phenyl)-3-chloro-5-thioxo-1,4,5,6-tetrahydro-pyrrolo[3,2-c]pyrazol-6-yl]-phenyl}-acetamide
116 Methanesulfonic acid 4-[2-chloro-4-(4-methanesulfonyloxy-phenyl)-5-thioxo-5,6-dihydro-4H-furo[2,3-b]pyrrol-4-yl]-phenyl ester
117 6-Chloro-7-cyclopropyl-3,3-bis-(4-hydroxy-phenyl)-1,3-dihydro-indol-2-one
118 6-Chloro-7-cyclopropyl-3,3-bis-(4-hydroxy-phenyl)-1,3-dihydro-pyrrolo[3,2-c]pyridin-2-one
119 6-Chloro-3,3-bis-(4-hydroxy-phenyl)-7-trifluoromethyl-1,3-dihydro-indol-2-one
120 6-Chloro-3,3-bis-(4-hydroxy-phenyl)-7-trifluoromethyl-1,3-dihydro-pyrrolo[3,2-c]pyridin-2-one
121 6-Chloro-7-cyclopropoxy-3,3-bis-(4-hydroxy-phenyl)-1,3-dihydro-indol-2-one
122 6-Chloro-7-cyclopropoxy-3,3-bis-(4-hydroxy-phenyl)-1,3-dihydro-pyrrolo[3,2-c]pyridin-2-one
123 6-(4-Fluoro-phenoxy)-3,3-bis-(4-hydroxy-phenyl)-7-trifluoromethyl-1,3-dihydro-indol-2-one
124 Acetic acid 4-[3-(4-acetoxy-phenyl)-6-chloro-7-cyclopropyl-2-oxo-2,3-dihydro-1.H-indol-3yl]-phenyl ester
125 Acetic acid 4-[3-(4-acetoxy-phenyl)-6-chloro-7-cyclopropyl-2-oxo-2,3-dihydro-1H-pyrrolo[3,2-c]pyridin-3-yl]-phenyl ester
126 Acetic acid 4-[3-(4-acetoxy-phenyl)-6-chloro-2-oxo-7-trifluoromethyl-2,3-dihydro-1H-indol-3yl]-phenyl ester
127 Acetic acid 4-[3-(4-acetoxy-phenyl)-6-chloro-2-oxo-7-trifluoromethyl-2,3-dihydro-1H-pyrrolo[3,2-c]pyridin-3-yl]-phenyl ester
128 Acetic acid 4-[3-(4-acetoxy-phenyl)-6-chloro-7-cyclopropoxy-2-oxo-2,3-dihydro-1H-indol-3-yl]phenyl ester
129 Acetic acid 4-[3-(4-acetoxy-phenyl)-6-chloro-7-cyclopropoxy-2-oxo-2,3-dihydro-1H-pyrrolo[3,2-c]pyridin-3-yl]-phenyl ester
130 Acetic acid 4-[3-(4-acetoxy-phenyl)-6-(4-fluoro-phenoxy)-2-oxo-7-trifluoromethyl-2,3-dihydro-1H-indol-3-yl]-phenyl ester
131 Dimethylamino-acetic acid 4-{6-chloro-7-cyclopropyl-3-[4-(2-dimethylamino-acetoxy)-phenyl]-2-oxo-2,3-dihydro-1H-indol-3-yl}-phenyl ester
132 Dimethylamino-acetic acid 4-{6-chloro-7-cyclopropyl-3-[4-(2-dimethylamino-acetoxy)-phenyl]-2-oxo-2,3-dihydro-1H-pyrrolo[3,2-c]pyridin-3-yl}-phenyl ester
133 Dimethylamino-acetic acid 4-{6-chloro-3-[4-(2-dimethylamino-acetoxy)-phenyl]-7-methyl-2oxo-2,3-dihydro-1H-indol-3-yl}-phenyl ester
134 6-Chloro-3,3-bis-(4-hydroxy-phenyl)-7-trifluoromethoxy-1,3-dihydro-indol-2-one
135 Acetic acid 4-[3-(4-acetoxy-phenyl)-6-chloro-2-oxo-7-trifluoromethoxy-2,3-dihydro-1H-indol-3-yl]-phenyl ester
136 Dimethylamino-acetic acid 4-{6-chloro-3-[4-(2-dimethylamino-acetoxy)-phenyl]-2-oxo-7-trifluoromethoxy-2,3-dihydro-1H-indol-3-yl}-phenyl ester
137 6-Chloro-4-fluoro-3,3-bis-(4-hydroxy-phenyl)-7-methyl-1,3-dihydro-indol-2-one
138 3-Chloro-7,7-bis-(4-hydroxy-phenyl)-4-methyl-5,7-dihydro-pyrrolo[3,2-c]pyridazin-6-one
139 Acetic acid 4-[3-(4-acetoxy-phenyl)-6-chloro-4-fluoro-7-methyl-2-oxo-2,3-dihydro-1H-indol-3yl]-phenyl ester
140 Acetic acid 4-[3-(4-acetoxy-phenyl)-6-chloro-4,7-dimethyl-2-oxo-2,3-dihydro-1H-indol-3-yl]-phenyl ester
141 Acetic acid 4-[7-(4-acetoxy-phenyl)-3-chloro-4-methyl-6-oxo-6,7-dihydro-5H-pyrrolo[3,2c]pyridazin-7-yl]-phenyl ester
142 6-Chloro-4,5-difluoro-3,3-bis-(4-hydroxy-phenyl)-7-methyl-1,3-dihydro-indol-2-one
143 Acetic acid 4-[3-(4-acetoxy-phenyl)-6-chloro-4,5-difluoro-7-methyl-2-oxo-2,3-dihydro-1H-indol-3-yl]-phenyl ester
144 3,3-Bis-(4-hydroxy-phenyl)-3,6,7,8-tetrahydro-1H-1-aza-as-indacen-2-one
145 3,3-Bis-(4-hydroxy-phenyl)-1,3,6,7,8,9-hexahydro-benzo[g]indol-2-one
146 3,3-Bis-(4-hydroxy-phenyl)-7-trifluoromethyl-1,3-dihydro-indol-2-one
147 7-Chloro-3,3-bis-(4-hydroxy-phenyl)-1,3-dihydro-indol-2-one
148 3,3-Bis-(4-hydroxy-phenyl)-2-oxo-2,3-dihydro-1H-indole-7-carbonitrile
149 7-Ethyl-3,3-bis-(4-hydroxy-phenyl)-1,3-dihydro-indol-2-one
150 3,3-Bis-(4-hydroxy-phenyl)-7-morpholin-4-yl-1,3-dihydro-indol-2-one
151 3,3-Bis-(4-hydroxy-phenyl)-7-isopropyl-1,3-dihydro-indol-2-one
152 7-tert-Butyl-3,3-bis-(4-hydroxy-phenyl)-1,3-dihydro-indol-2-one
153 3,3-Bis-(4-hydroxy-phenyl)-2-oxo-2,3-dihydro-1H-indole-7-carboxylic acid dimethylamide
154 3,3-Bis-(4-hydroxy-phenyl)-7-(4-methyl-piperazine-1-carbonyl)-1,3-dihydro-indol-2-one
155 3,3-Bis-(4-hydroxy-phenyl)-2-oxo-2,3-dihydro-1H-indole-5-carboxylic acid
156 3,3-Bis-(4-hydroxy-phenyl)-2-oxo-2,3-dihydro-1H-indole-5-carboxylic acid dimethylamide
157 3,3-Bis-(4-hydroxy-phenyl)-5-(morpholine-4-carbonyl)-1,3-dihydro-indol-2-one
158 3,3-Bis-(4-hydroxy-phenyl)-4-methoxy-1,3-dihydro-indol-2-one
159 3,3-Bis-(4-hydroxy-phenyl)-6-methoxy-1,3-dihydro-indol-2-one
160 3,3-Bis-(4-hydroxy-phenyl)-5-(4-methyl-piperazine-1-carbonyl)-1,3-dihydro-indol-2-one
161 6-Chloro-3,3-bis-(4-mercapto-phenyl)-7-methyl-1,3-dihydro-indol-2-one
162 N-{4-[3-(4-Acetylamino-phenyl)-7-methyl-2-oxo-2,3-dihydro-1H-indol-3-yl]-phenyl}-acetamide
163 3,3-Bis-(4-hydroxy-phenyl)-7-(3-methoxy-prop-1-ynyl-1,3-dihydro-indol-2-one
164 3,3-Bis-(4-hydroxy-phenyl)-7-pyridin-3-yl-1,3-dihydro-indol-2-one
165 7-Bromo-3,3-bis-(4-hydroxy-phenyl)-1,3-dihydro-indol-2-one
166-Chloro-3,3-bis-(4-methanesulfonyl-phenyl)-7-methyl-1,3-dihydro-indol-2-one
167 6,6-Bis-(4-hydroxy-phenyl)-4,6-dihydro-pyrrolo[3,2-d]thiazol-5-one
168 6,6-Bis-(4-hydroxy-phenyl)-2-methyl-4,6-dihydro-pyrrolo[3,2-d]thiazol-5-one
169 6,6-Bis-(4-hydroxy-phenyl)-2-isopropyl-4,6-dihydro-pyrrolo[3,2-d]thiazol-5-one
170 2-Chloro-6,6-bis-(4-hydroxy-phenyl)-4,6-dihydro-pyrrolo[3,2-d]thiazol-5-one
171 4,4-Bis-(4-hydroxy-phenyl)-4,6-dihydro-pyrrolo[3,2-d]isothiazol-5-one
172 3,3-Bis-(4-hydroxy-phenyl)-7-methyl-1,3-dihydro-pyrrolo[2,3-c]pyridin-2-one
173 3,3-Bis-(4-hydroxy-phenyl)-7-methyl-1,3-dihydro-pyrrolo[3,2-b]pyridin-2-one
174 3,3-Bis-(4-fluoro-phenyl)-7-methyl-1,3-dihydro-pyrrolo[3,2-b]pyridin-2-one
175 3,3-Bis-(4-fluoro-phenyl)-7-methyl-1,3-dihydro-pyrrolo[3,2-c]pyridin-2-one
176 3,3-Bis-(4-fluoro-phenyl)-7-isopropyl-1,3-dihydro-pyrrolo[3,2-c]pyridin-2-one
177 3,3-Bis-(4-hydroxy-phenyl)-3,6,7,8-tetrahydro-1H-1,5-diaza-as-indacen-2-one
178 3,3-Bis-(4-hydroxy-phenyl)-3,6,7,8-tetrahydro-1H-1,4-diaza-as-indacen-2-one
179 3,3-Bis-(4-hydroxy-phenyl)-1,3,6,7,8,9-hexahydro-pyrrolo[3,2-c]quinolin-2-one
180 3,3-Bis-(4-hydroxy-phenyl)-1,3,6,7,8,9-hexahydro-pyrrolo[3,2-c]isoquinolin-2-one
181 5-Fluoro-3,3-bis-(4-hydroxy-phenyl)-3,6,7,8-tetrahydro-1H-1-aza-as-indacen-2-one
182 7-Ethyl-5-fluoro-3,3-bis-(4-hydroxy-phenyl)-1,3-dihydro-indol-2-one
183 3,3-Bis-(4-hydroxy-phenyl)-1,3,6,8-tetrahydro-7-oxa-1-aza-as-indacen-2-one
184 3,3-Bis-(4-hydroxy-phenyl)-1,3,7,8-tetrahydro-6-oxa-1-aza-as-indacen-2-one
185 3,3-Bis-(4-hydroxy-phenyl)-1,6,7,9-tetrahydro-3H-8-oxa-1-aza-cyclopenta[a]naphthalen-2-one
186 3,3-Bis-(4-hydroxy-phenyl)-1,7,8,9-tetrahydro-3H-pyrano[2,3-g]indol-2-one
187 3,3-Bis-(4-hydroxy-phenyl)-7-methyl-3,6,7,8-tetrahydro-1H-1,7-diaza-as-indacen-2-one
188 3,3-Bis-(4-hydroxy-phenyl)-7-methyl-1,3,7,8-tetrahydro-1,7-diaza-as-indacene-2,6-dione
189 3,3-Bis-(4-hydroxy-phenyl)-7,8,8-trimethyl-1,3,7,8-tetrahydro-1,7-diaza-as-indacene-2,6-dione
190 3,3-Bis-(4-hydroxy-phenyl)-5-iodo-1,3-dihydro-indol-2-one
191 5-Amino-3,3-bis-(4-hydroxy-phenyl)-1,3-dihydro-indol-2-one
192 5-Amino-3,3-bis-(4-hydroxy-phenyl)-7-methyl-1,3-dihydro-indol-2-one
193 6-Bromo-3,3-bis-(4-hydroxy-phenyl)-7-methyl-1,3-dihydro-indol-2-one
194 7-Fluoro-3,3-bis-(4-hydroxy-phenyl)-1,3-dihydro-indol-2-one
195 3,3-Bis-(4-hydroxy-phenyl)-7-methoxy-1,3-dihydro-indol-2-one
196 4,7-Dichloro-3,3-bis-(4-hydroxy-phenyl)-1,3-dihydro-indol-2-one
197 6-Chloro-3,3-bis-(4-hydroxy-phenyl)-1,7-dimethyl-1,3-dihydro-indol-2-one
198 6-Chloro-3,3-bis-(4-fluoro-phenyl)-7-methyl-1,3-dihydro-indol-2-one
199 3,3-Bis-(4-hydroxy-phenyl)-7-(morpholine-4-carbonyl)-1,3-dihydro-indol-2-one
200 3,3-Bis-(4-hydroxy-phenyl)-1,3-dihydro-pyrrolo[2,3-d]pyridin-2-one
201 N-{4-[6-Chloro-3-(4-methanesulfonylamino-phenyl)-7-methyl-2-oxo-2,3-dihydro-1H-indol-3-yl]-phenyl}-methanesulfonamide
202 3,3-Bis-(4-hydroxy-phenyl)-4,7-dimethyl-1,3-dihydro-indol-2-one
203 3,3-Bis-(4-hydroxy-phenyl)-7-iodo-1,3-dihydro-indol-2-one
204 3,3-Bis-(4-hydroxy-phenyl)-7-pyridin-4-yl-1,3-dihydro-indol-2-one
205 Acetic acid 4-[3-(4-acetoxy-phenyl)-6-chloro-7-methyl-2-oxo-2 3-dihydro-1H-indol-3-yl]-phenyl ester
206 3,3-Bis-(4-hydroxy-phenyl)-5-phenyl-1,3-dihydro-indol-2-one
207 3,3-Bis-(4-hydroxy-phenyl)-7-thiophen-2-yl-1,3-dihydro-indol-2-one
208 3,3-Bis-(4-hydroxy-phenyl)-5-pyridin-4-yl-1,3-dihydro-indol-2-one
209 3,3-Bis-(4-hydroxy-phenyl)-5-thiophen-2-yl-1,3-dihydro-indol-2-one
210 5,7-Difluoro-3,3-bis-(4-hydroxy-phenyl)-1,3-dihydro-indol-2-one
211 6-Fluoro-3,3-bis-(4-hydroxy-phenyl)-7-methyl-1,3-dihydro-indol-2-one
212 3,3-Bis-(4-hydroxy-phenyl)-6-methoxy-7-methyl-1,3-dihydro-indol-2-one
213 6,7-Difluoro-3,3-bis-(4-hydroxy-phenyl)-1,3-dihydro-indol-2-one
214 6-Chloro-7-fluoro-3,3-bis-(4-hydroxy-phenyl)-1,3-dihydro-indol-2-one
215 5-Fluoro-3,3-bis-(4-hydroxy-phenyl)-7-methyl-1,3-dihydro-indol-2-one
216 3,3-Bis-(4-hydroxy-phenyl)-5-methoxy-7-methyl-1,3-dihydro-indol-2-one
217 3,3-Bis-(4-hydroxy-phenyl)-1,3-dihydro-pyrrolo[2,3-b]pyridin-2-one
218 7-Chloro-3,3-bis-(4-hydroxy-phenyl)-4-methoxy-1,3-dihydro-indol-2-one
219 6-Fluoro-3,3-bis-(4-hydroxy-phenyl)-1,3-dihydro-indol-2-one
220 N-[3,3-Bis-(4-hydroxy-phenyl)-2-oxo-2,3-dihydro-indol-1-yl]-acetamide
221 5-[3,3-Bis-(4-hydroxy-phenyl)-7-methyl-2-oxo-2,3-dihydro-1H-indol-6-yloxy]-pentanoic acid methyl ester
222 5-[3,3-Bis-(4-hydroxy-phenyl)-7-methyl-2-oxo-2,3-dihydro-1H-indol-6-yloxy]-pentanoic acid
223 5-[3,3-Bis-(4-hydroxy-phenyl)-7-methyl-2-oxo-2,3-dihydro-1H-indol-5-yloxy]-pentanoic acid methyl ester
224 5-[3,3-Bis-(4-hydroxy-phenyl)-7-methyl-2-oxo-2,3-dihydro-1H-indol-5-yloxy]-pentanoic acid
225 7-Chloro-6-fluoro-3,3-bis-(4-hydroxy-phenyl)-1,3-dihydro-indol-2-one.

29: The method according to claim 1, wherein the medicament further comprises one or more other chemotherapeutic agents.

30. (canceled)

31: A compound of the general formula (I) 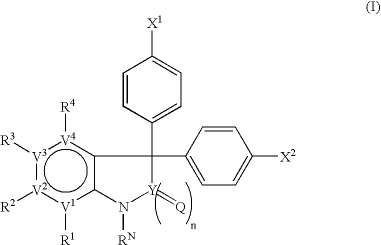
as defined in claim 1, with the proviso that the compound is not one selected from
3,3-bis-(4-hydroxy-phenyl)-1,3-dihydro-indol-2-one,
3,3-bis-(4-hydroxy-phenyl)-7-methyl-1,3-dihydro-indol-2-one;
3,3-bis-(4-hydroxy-phenyl)-4,5-dimethyl-1,3-dihydro-indol-2-one;
3,3-bis-(4-hydroxy-phenyl)-5,7-dimethyl-1,3-dihydro-indol-2-one;
5-bromo-3,3-bis-(4-hydroxy-phenyl)-1,3-dihydro-indol-2-one;
5-chloro-3,3-bis-(4-hydroxy-phenyl)-1,3-dihydro-indol-2-one;
3,3-bis-(4-hydroxy-phenyl)-5-methoxy-1,3-dihydro-indol-2-one;
3,3-bis-(4-hydroxy-phenyl)-5-methyl-1,3-dihydro-indol-2-one;
6-chloro-3,3-bis-(4-hydroxy-phenyl)-7-methyl-1,3-dihydro-indol-2-one;
acetic acid 4-[3-(4-acetoxy-phenyl)-2-oxo-2,3-dihydro-1H-indol-3-yl]-phenyl ester; and
acetic acid 4-[3-(4-acetoxy-phenyl)-5-methyl-2-oxo-2,3-dihydro-1H-indol-3-yl]-phenyl ester.

32: A 3,3-Diphenyl-1,3-dihydro-indol-2-one type compound of the formula (II) 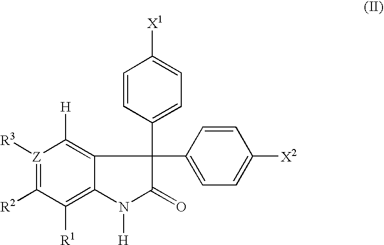
as defined in claim 24, with the proviso that the compound is not one selected from:
3,3-bis-(4-hydroxy-phenyl)-1,3-dihydro-indol-2-one,
3,3-bis-(4-hydroxy-phenyl)-7-methyl-1,3-dihydro-indol-2-one;
3,3-bis-(4-hydroxy-phenyl)-4,5-dimethyl-1,3-dihydro-indol-2-one
3,3-bis-(4-hydroxy-phenyl)-5,7-dimethyl-1,3-dihydro-indol-2-one;
5-bromo-3,3-bis-(4-hydroxy-phenyl)-1,3-dihydro-indol-2-one;
5-chloro-3,3-bis-(4-hydroxy-phenyl)-1,3-dihydro-indol-2-one;
3,3-bis-(4-hydroxy-phenyl)-5-methoxy-1,3-dihydro-indol-2-one;
3,3-bis-(4-hydroxy-phenyl)-5-methyl-1,3-dihydro-indol-2-one;
6-chloro-3,3-bis-(4-hydroxy-phenyl)-7-methyl-1,3-dihydro-indol-2-one;
acetic acid 4-[3-(4-acetoxy-phenyl)-2-oxo-2,3-dihydro-1H-indol-3-yl]-phenyl ester; and
acetic acid 4-[3-(4-acetoxy-phenyl)-5-methyl-2-oxo-2,3-dihydro-1H-indol-3-yl]-phenyl ester.

33: A pharmaceutical composition comprising a compound as defined in claim 1 and a pharmaceutically acceptable carrier.

34: A 3,3-Diphenyl-1,3-dihydro-indol-2-one type compound of the formula (II) 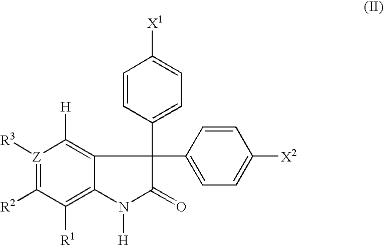
as defined in claim 25, with the proviso that the compound is not one selected from:
3,3-bis-(4-hydroxy-phenyl)-1,3-dihydro-indol-2-one,
3,3-bis-(4-hydroxy-phenyl)-7-methyl-1,3-dihydro-indol-2-one;
3,3-bis-(4-hydroxy-phenyl)-4,5-dimethyl-1,3-dihydro-indol-2-one;
3,3-bis-(4-hydroxy-phenyl)-5,7-dimethyl-1,3-dihydro-indol-2-one;
5-bromo-3,3-bis-(4-hydroxy-phenyl)-1,3-dihydro-indol-2-one;
5-chloro-3,3-bis-(4-hydroxy-phenyl)-1,3-dihydro-indol-2-one;
3,3-bis-(4-hydroxy-phenyl)-5-methoxy-1,3-dihydro-indol-2-one;
3,3-bis-(4-hydroxy-phenyl)-5-methyl-1,3-dihydro-indol-2-one;
6-chloro-3,3-bis-(4-hydroxy-phenyl)-7-methyl-1,3-dihydro-indol-2-one;
acetic acid 4-[3-(4-acetoxy-phenyl)-2-oxo-2,3-dihydro-1H-indol-3-yl]-phenyl ester; and
acetic acid 4-[3-(4-acetoxy-phenyl)-5-methyl-2-oxo-2,3-dihydro-1H-indol-3-yl]-phenyl ester.

35: A 3,3-Diphenyl-1,3-dihydro-indol-2-one type compound of the formula (II) 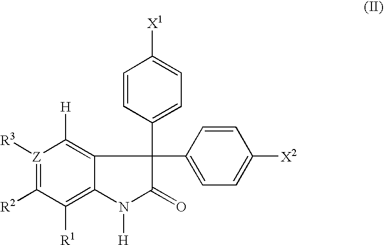
as defined in claim 26, with the proviso that the compound is not one selected from:
3,3-bis-(4-hydroxy-phenyl)-1,3-dihydro-indol-2-one,
3,3-bis-(4-hydroxy-phenyl)-7-methyl-1,3-dihydro-indol-2-one;
3,3-bis-(4-hydroxy-phenyl)-4,5-dimethyl-1,3-dihydro-indol-2-one;
3,3-bis-(4-hydroxy-phenyl)-5,7-dimethyl-1,3-dihydro-indol-2-one;
5-bromo-3,3-bis-(4-hydroxy-phenyl)-1,3-dihydro-indol-2-one;
5-chloro-3,3-bis-(4-hydroxy-phenyl)-1,3-dihydro-indol-2-one;
3,3-bis-(4-hydroxy-phenyl)-5-methoxy-1,3-dihydro-indol-2-one;
3,3-bis-(4-hydroxy-phenyl)-5-methyl-1,3-dihydro-indol-2-one;
6-chloro-3,3-bis-(4-hydroxy-phenyl)-7-methyl-1,3-dihydro-indol-2-one;
acetic acid 4-[3-(4-acetoxy-phenyl)-2-oxo-2,3-dihydro-1H-indol-3-yl]-phenyl ester; and
acetic acid 4-[3-(4-acetoxy-phenyl)-5-methyl-2-oxo-2,3-dihydro-1H-indol-3-yl]-phenyl ester.

36: A 3,3-Diphenyl-1,3-dihydro-indol-2-one type compound of the formula (II) 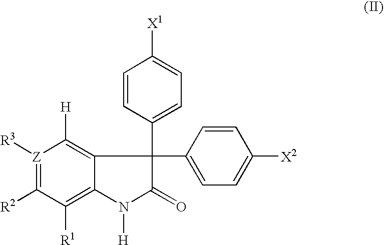
as defined in claim 27, with the proviso that the compound is not one selected from:
3,3-bis-(4-hydroxy-phenyl)-1,3-dihydro-indol-2-one,
3,3-bis-(4-hydroxy-phenyl)-7-methyl-1,3-dihydro-indol-2-one;
3,3-bis-(4-hydroxy-phenyl)-4,5-dimethyl-1,3-dihydro-indol-2-one;
3,3-bis-(4-hydroxy-phenyl)-5,7-dimethyl-1,3-dihydro-indol-2-one;
5-bromo-3,3-bis-(4-hydroxy-phenyl)-1,3-dihydro-indol-2-one;
5-chloro-3,3-bis-(4-hydroxy-phenyl)-1,3-dihydro-indol-2-one;
3,3-bis-(4-hydroxy-phenyl)-5-methoxy-1,3-dihydro-indol-2-one;
3,3-bis-(4-hydroxy-phenyl)-5-methyl-1,3-dihydro-indol-2-one;
6-chloro-3,3-bis-(4-hydroxy-phenyl)-7-methyl-1,3-dihydro-indol-2-one;
acetic acid 4-[3-(4-acetoxy-phenyl)-2-oxo-2,3-dihydro-1H-indol-3-yl]-phenyl ester; and
acetic acid 4-[3-(4-acetoxy-phenyl)-5-methyl-2-oxo-2,3-dihydro-1H-indol-3-yl]-phenyl ester.

37: A 3,3-Diphenyl-1,3-dihydro-indol-2-one type compound of the formula (II) 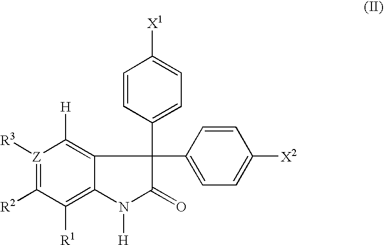
as defined in claim 28, with the proviso that the compound is not one selected from:
3,3-bis-(4-hydroxy-phenyl)-1,3-dihydro-indol-2-one,
3,3-bis-(4-hydroxy-phenyl)-7-methyl-1,3-dihydro-indol-2-one;
3,3-bis-(4-hydroxy-phenyl)-4,5-dimethyl-1,3-dihydro-indol-2-one;
3,3-bis-(4-hydroxy-phenyl)-5,7-dimethyl-1,3-dihydro-indol-2-one;
5-bromo-3,3-bis-(4-hydroxy-phenyl)-1,3-dihydro-indol-2-one;
5-chloro-3,3-bis-(4-hydroxy-phenyl)-1,3-dihydro-indol-2-one;
3,3-bis-(4-hydroxy-phenyl)-5-methoxy-1,3-dihydro-indol-2-one;
3,3-bis-(4-hydroxy-phenyl)-5-methyl-1,3-dihydro-indol-2-one;
6-chloro-3,3-bis-(4-hydroxy-phenyl)-7-methyl-1,3-dihydro-indol-2-one;
acetic acid 4-[3-(4-acetoxy-phenyl)-2-oxo-2,3-dihydro-1H-indol-3-yl]-phenyl ester; and
acetic acid 4-[3-(4-acetoxy-phenyl)-5-methyl-2-oxo-2,3-dihydro-1H-indol-3-yl]-phenyl ester.
